(19) United States
(12) Patent Application Publication
(10) Pub. No.: US 2017/0055499 A1
(43) Pub. Date: Mar. 2, 2017

(54) TATTOO AND TATTOO APPLICATOR FOR ANIMAL LIFECYCLE MONITORING

(71) Applicant: EMPIRE TECHNOLOGY DEVELOPMENT LLC, Wilmington, DE (US)

(72) Inventors: George Charles PEPPOU, Horsby Heights (AU); Michael Keoni MANION, Seattle, WA (US); Benjamin William MILLAR, Rosebery (AU)

(21) Appl. No.: 14/904,089

(22) PCT Filed: May 21, 2015
(86) PCT No.: PCT/US15/32036

§ 371 Date: Jan. 9, 2016

Related U.S. Application Data
(60) Provisional application No. 62/001,371, filed on May 21, 2014.
(60) Provisional application No. 62/001,541, filed on May 21, 2014.

(51) Int. Cl. A01K 29/00; A61D 17/00; A61B 5/145; A61B 5/01; A01K 11/00; A61B 5/00

(52) U.S. Cl. CPC A01K 29/005; A01K 11/005; A61B 5/0071; A61B 5/14532; A61B 5/14539; A61B 5/01; A61B 5/4875; A61D 17/00; A61B 2503/40

(57) ABSTRACT

In some examples, a method to monitor an animal may include determining a physiological status of the animal based on a tattoo that includes intelligent ink injected into skin of the animal. The method may also include recording the physiological status of the animal. The method may also include, in response to the physiological status indicating a physiological event or condition, triggering an alert to address the physiological event or condition.

### BACKGROUND

Unless otherwise indicated herein, the materials described herein are not prior art to the claims in the present application and are not admitted to be prior art by inclusion in this section.

The health of livestock used in the meat or dairy industry or for breeding may determine the quality and value of the end product and information about the health of the livestock may not be readily available. For example, the health and stress state of animals raised for slaughter may not be known until post-slaughter. Some animals may be dark cutting due to stress prior to slaughter, which stress may arise from transportation, rough handling, changing weather conditions such as cold fronts, or other factors that cause the animal to draw on its glycogen reserves before slaughter. In dark cutting animals, there may be relatively less glycogen available to be converted to lactic acid in the animal post-slaughter and the color of its meat may therefore be darker than normal. Such dark cutting meat may be considered to be lower grade and less valuable than non-dark cutting meat.

### SUMMARY

Technologies described herein generally relate to tattoos for animal lifecycle monitoring.

In some examples, a method to monitor an animal may include determining a physiological status of the animal based on a tattoo that includes intelligent ink injected into skin of the animal. The method may also include recording the physiological status of the animal. The method may also include, in response to the physiological status indicating a physiological event or condition, triggering an alert to address the physiological event or condition.

In some examples, a system to monitor an animal may include a tattoo reader, a processor, a computer-readable medium, and a health monitor application. The tattoo reader may be configured to measure an optical property of an intelligent ink injected into skin of the animal as a tattoo and to generate a current value of the measured optical property. The processor may be communicatively coupled to the tattoo reader. The computer-readable medium may be communicatively coupled to the processor. The health monitor application may include computer instructions executable by the processor to perform operations. The operations may include determining a physiological status of the animal based on the current value of the measured optical property. The operations may also include recording the physiological status of the animal. The operations may also include, in response to the physiological status indicating a physiological event or condition, triggering an alert to address the physiological event or condition.

In some examples, a computer-readable medium that includes computer-readable instructions stored thereon is described. In response to execution by a processor, the computer-readable instructions may cause the processor to perform or may cause the processor to control performance of operations comprising determining a physiological status of an animal based on a tattoo that includes intelligent ink injected into skin of the animal. The operations may also include recording the physiological status of the animal. The operations may also include, in response to the physiological status indicating a physiological event or condition, triggering an alert to address the physiological event or condition.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings. In the drawings:

FIG. 1A is a diagram of an example system to monitor one or more animals;

FIG. 1B is a diagram of another example system to monitor the one or more animals of FIG. 1A;

FIG. 2 illustrates an example animal and placement of one or more tattoos with intelligent ink on the animal;

FIGS. 3A-3C illustrate an example animal ear, tattoo applicator, and tattoos that may be applied to the animal ear;

FIG. 4 is a flowchart of an example method to monitor an animal;

FIG. 5 is a block diagram of an example tattoo applicator;

FIG. 6A illustrates an example configuration of some elements of a tattoo applicator with needles in a retracted state;

FIG. 6B illustrates the tattoo applicator of FIG. 6A with the needles in an unretracted state;

FIG. 6C illustrates an example of an ink roller of the tattoo applicator of FIGS. 6A and 6B;

FIG. 7A illustrates another example configuration of some elements of a tattoo applicator with needles in a retracted state;

FIG. 7B illustrates the tattoo applicator of FIG. 7A with the needles in an unretracted state;

FIG. 7C illustrates an example of an ink pad of the tattoo applicator of FIGS. 7A and 7B;

FIG. 8A illustrates another example configuration of some elements of a tattoo applicator with needles in a retracted state;

FIG. 8B illustrates the tattoo applicator of FIG. 8A with the needles in an unretracted state;

FIG. 9A illustrates an example pattern selection mechanism of a tattoo applicator;

FIG. 9B illustrates a bottom-view of a tattoo applicator that includes the pattern selection mechanism of FIG. 9A,

FIGS. 10A and 10B illustrate an example configuration of some elements of another tattoo applicator;

FIG. 10C illustrates another example configuration of the tattoo applicator of FIGS. 10A and 10B;

FIG. 11 is a block diagram illustrating an example computing device that is arranged to monitor an animal and/or control a tattoo applicator;

FIG. 12 illustrates an example sensor that includes a PDA vesicle conjugated with multiple antigen receptors;

FIGS. 13A-13C depict operation of sensors injected into dermis of an animal;

FIGS. 14A and 14B depict injection of a tattoo ink into skin of an animal; and

FIG. 15 illustrates an example sensor that includes leuco dye,

all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and computer program products that generally relate to monitoring animal health based on tattoos that include intelligent ink and are applied to animals. The tattoos that include intelligent ink may be implemented in a variety of use cases other than monitoring animal health, some of which are described below. This disclosure is also generally drawn, inter alia, to methods, apparatus, and systems related to applying tattoos. A description of various methods, apparatus, systems, and devices associated with monitoring animal health based on tattoos and associated with applying tattoos will be given before referring to the drawings.

The term “tattoo” as used herein may include a pattern in skin produced by injecting ink and/or other ink elements into the skin. Tattoos described herein for monitoring animal health may be formed by tattoo inks that include one or more ink elements. Each ink element may include any of a variety of substances that may be applied at or into an injection site on skin of an animal. The area of the skin at which the tattoo is formed may be referred to as an injection site, and ink elements may be injected into the skin with or without penetration of the skin by one or more needles or microneedles. For example, ink elements may be injected to one or more layers or depths of the skin at the injection site by needles or microneedles that penetrate the skin to the layers or depths of the skin. As another example, ink elements may be injected to one or more layers or depths of the skin at the injection site by applying the ink elements to a surface of the skin and allowing the ink elements to solvate through the surface of the skin to one or more layers or depths of the skin without the skin being penetrated by needles or microneedles.

The layer or depth of the skin to which each ink element is injected may include epidermis, dermis, or subcutis of the skin, substructures of the epidermis such as stratum lucidum, stratum granulosum, stratum spinosum, stratum mucosum, stratum germinativum, stratum corneum, or other substructure of the epidermis, particular regions of the dermis such as a papillary region, reticular region, or other region of the dermis, or other particular layers, substructures, or regions of the skin. Each of the ink elements may include an intelligent ink, a non-intelligent ink, a factor, an agent, or a support element, various examples of which are described below.

In some embodiments, the intelligent inks and/or other ink elements may be developed by adaptation of in vitro assay technology for use in vivo with a visible or optical detection result. The adaptation may also include stabilizing the assays for mid term (e.g., a month or more) or long term (e.g., a year or more) residence in vivo. Alternatively or additionally, the intelligent inks or other ink elements may be developed, ab initio, as in vivo assays. The intelligent inks and/or other ink elements included in the in vivo assays may be sensed, transduced, and displayed through the resulting tattoos.

In some embodiments, intelligent ink may generally be configured to respond to a particular target of physiology of the animal, a part of the animal, or a compartment of the animal, which may be referred to as a target marker. Alternately or additionally, intelligent ink may include one or more sensors that have a variable optical property that may be indicative of a presence or absence of a target antigen or other target marker in the animal. The variable optical property may include a color, fluorescence, or other optical property that may have one value or range of values in an absence of the target antigen or other target marker and another value or range of values in an a presence of the target antigen or other target marker. The target marker may include glucose or a glucose level of the animal, water or a hydration level of the animal, a temperature of the animal, a pH of the animal, a hormone or a level of the hormone in the animal, a particular pathogen in the animal, a particular antigen in the animal, salts, minerals, vitamins, sugars, carbohydrates, lipids, phospholipids, nucleic acids, polynucleotides, proteins, antibodies, immunoregulatory molecules, disease markers, or other target markers that may be indicative of a physiological condition or event in the animal.

In some embodiments, intelligent inks may be configured to respond to antigens in an animal may include ultraviolet (UV)-irradiated colored polydiacetylene (PDA) vesicles coupled with antibodies or other antigen receptors. The antigens to which the intelligent ink can be configured to respond to, may be specific to particular hormones or may otherwise be indicative of hormone levels of the particular hormones, such as cortisol or progesterone levels, in the animal. Accordingly, the antigens may include hormone-specific antigens and the antibodies conjugated to the PDA vesicles may include hormone-specific antibodies. For example, the antibodies may include cortisol-specific antibodies to detect cortisol-specific antigens, or progesterone specific antibodies to detect progesterone-specific antigens.

In various embodiments, the PDA vesicles coupled with hormone-specific antibodies may be referred to as sensors. A distinct color change may be produced on the PDA vesicles when the hormone-specific antibodies are exposed to a corresponding hormone-specific antigen in solution. Biologically-produced hormone-specific antibodies can be conjugated to the PDA vesicles to produce the intelligent ink. A color change of the intelligent ink from blue to red in the presence of the hormone-specific antigens may be clear and sensitive. The color change may be due to immunoreaction at a surface of the PDA vesicle, which may result in a conformational change in the PDA vesicle. The color change in this example may be visible to the naked eye from concentrations as low as 100 nanogram per milliliter (ng/mL), 10 ng/mL, or 1 ng/mL. Concentrations as low as 100 ng/mL, 10 ng/mL, or 1 ng/mL may be sufficiently sensitive to be used as a threshold sensor for blood concentration of cortisol or progesterone, for example. However virtually any antigen could be detected using such an intelligent ink by conjugating an appropriate antibody, antibody mimetic, receptor molecule, or other antigen receptor that is configured to bind a target antigen or other target marker to the PDA vesicle. Tuning the sensitivity of these systems may be accomplished through addition of various amounts of lipids into membranes of the PDA vesicle: types and concentrations of lipids can alter the sensitivity.

In some embodiments, the tattoo ink that includes an intelligent ink with PDA vesicles may be delivered in a carrier, such as phosphate buffered saline (PBS). In some embodiments, the carrier may provide flow and/or may match conditions (e.g., pH and osmolarity) of an injection site.

In some embodiments, the intelligent ink that includes such sensors can be injected in the skin to form the tattoo, the intelligent ink may alter color from blue to red in response to binding of the hormone-specific antigen to the hormone-specific antibodies conjugated to the PDA vesicles. The color change may be readable by the naked eye. It is also possible for the color change to be machine read. For example, an optical imager may include a digital camera to take a picture that a computer can process to determine the color(s) in the tattoo. Alternatively, the optical imager may include a spectrophotometer that can measure absorbance of light of the tattoo to determine what the color of the tattoo is at a given point in time. The determined color information can then be correlated with a unique identifier (ID) of the animal.

In some embodiments, PDA may alternate between a non-fluorescent state (e.g. when it is blue) to a fluorescent state (e.g. when it is red). As such, the intelligent ink in this example can also be read by measuring fluorescence of the intelligent ink. To measure the fluorescence, an appropriate excitation illumination may be provided, e.g., by a radiation source. When the PDA of the intelligent ink is red, exposure of the PDA to the excitation illumination may cause the PDA to fluoresce. The fluorescence may be measured using a fluorescence spectrophotometer or other suitable device.

The concentration of intelligent ink in various embodiments required to be visible to the naked eye and/or to be machine-read may depend on one or more factors, such as depth of the tattoo, type of animal or the injection site on its hide, area of the tattoo, whether the color or fluorescence is measured, the environment the tattoo may be read in as well as a variety of other factors. However, if the intelligent ink is to be read by the naked eye, a 1 square centimeter (cm) area being tattooed with the intelligent ink may include between 1 milligram (mg) and 100 mg of PDA vesicles, or between 40 mg and 100 mg, or between 80 mg and 100 mg. A size of the area being tattooed and a concentration of the intelligent ink in the area being tattooed may be adjusted, as needed.

In some embodiments, the intelligent ink may be stabilized in the hide of the animal for an extended time, e.g., for several months or even a year or more. In various embodiments, the PDA vesicles of the intelligent ink may be encapsulated in a semipermeable encapsulant to reduce their deterioration and resorption compared to non-encapsulated embodiments. Alginate encapsulation for xenotransplants affords a stable environment for material that would otherwise be attacked by a host's immune system and quickly removed. The alginate may be biocompatible and can be tuned to an appropriate molecular weight cutoff to ensure passage of molecules of interest (e.g., the hormone-specific antigens) into, and out of, the vicinity of the sensor. In a non-limiting example, a PDA vesicle which may be conjugated to a hormone-specific antibody that specifically binds cortisol, a molecular weight cutoff of about 1 kilodalton (kDa) may be sufficient to allow relatively free diffusion of cortisol while preventing ingress into a vicinity of the sensor of enzymes that have a molecular weight above the molecular weight cutoff. Such enzymes may include proteases or other enzymes that may degrade the hormone-specific antibody.

In some embodiments, the sensors may be encapsulated in a time-release encapsulant which may be semipermeable or impermeable. The time-release encapsulant may degrade over a period of time to eventually expose the sensor within. The duration of time may include a period of time on the order of seconds, minutes, hours, days, weeks, months, or some other duration. For example, the duration of time may include 24 hours, 30 days, 45 days, 90 days, or some other duration of time. In some embodiments, different sets of sensors in the intelligent ink may be encapsulated in time-release encapsulants of different durations of time to stagger exposure of the different sets of sensors over time. If the time-release encapsulant is semipermeable, the sensors may operate as described above while encapsulated. After degradation of the semipermeable time-release encapsulant, the sensors may be exposed to deterioration and resorption within the animal, which may eventually cause the tattoo with the intelligent ink, or at least some portion of the intelligent ink of the tattoo, to fade or disappear if the exposed intelligent ink is not otherwise stabilized within the hide of the animal. If the time-release encapsulant is impermeable, the sensors may generally be inactive while encapsulated within the impermeable time-release encapsulant. After degradation of the impermeable time-release encapsulant, the sensors may be activated to sense antigens or other compounds. The encapsulation of the sensors in time-release encapsulants may be used to cause automatic fading or disappearance of the tattoo over time, time-delayed activation of some or all of the sensors, or some other outcome.

In some embodiments, the intelligent ink may be embedded into capillary beds of the dermis to ensure significant perfusion in some embodiments, or at any other desired layer, depth, or structure of the animal's skin. Other ink elements, such as one or more factors described below, may be injected into the skin with or near the intelligent ink to increase perfusion of the sensors by encouraging growth of capillaries in the vicinity of the tattoo. The dermis may provide contact with capillary beds, while still being close enough to a surface of the animal's hide to ensure visibility of the sensors. The epidermis may provide a protective layer over the tattoo that aids in stabilizing it over time. Alternatively, the tattoo may be embedded more towards the epidermis, within the epidermis, or even beneath the dermis, such as within the hypodermis or subcutis. In some cases, the tattoo may be embedded into multiple layers within the animal's skin structure.

In some embodiments, tattoo inks that include the foregoing intelligent ink may also include one or more other ink elements to facilitate and/or improve measurement of a corresponding tattoo. For example, the tattoo ink may also include one or more factors, agents, and/or support structures. Two example factors include vascular endothelial growth factor (VEGF) and epidermal growth factor (EGF). VEGF may recruit blood vessels to the vicinity of the sensors after the tattoo is injected to increase or ensure exposure of the sensors to antigens or other markers when they are present in the blood stream. EGF may promote healing and reduce scar formation at the injection site, which may improve visibility of the sensors and/or the intelligent ink through the animal's skin. Agents may include opacity-enhancing agents, depigmentation agents such as monobenzone, depilatory agents such as thioglycolic acid, UV blocking agents such as titanate, or other agents to improve visibility of the sensors and/or the intelligent ink and/or to extend a life of the tattoo. Support structures may include hydrogel scaffolds, such as a crosslinked collagen hydrogel scaffold that may be infiltrated by epidermal cells or other cells after injection of the tattoo ink to heal and reduce scar tissue at the injection site.

In some embodiments, the various ink elements of the tattoo ink may be mixed together and/or delivered together at the injection site. Alternately or additionally, some ink elements may be delivered separately from other ink elements. Various tattoo applicators are described below that are capable of delivering different ink elements (or the same ink element) to different layers or depths of the skin. The particular layer or depth of the skin to which each ink element is delivered may depend on the ink element and its intended purpose, the location of the injection site on the animal's hide, and the type of animal, among potentially other criteria. For example, the above-described intelligent ink with PDA vesicles may be into to the dermis to contact the capillary beds of the animal's skin, depigmentation agents that effectively remove melanin or other pigments present at the injection site may be injected into the epidermis to act on melanocytes present there, while depilatory agents that remove hair may be applied to a surface of the skin or may be injected to a layer within the dermis or hypodermis where hair follicles reside.

Strategies for generating the many embodiments of intelligent inks and ink elements may utilize generally recognized as safe (GRAS) chemistry such that the intelligent inks and ink elements may be used in production animals (which may enter the food chain) and/or in humans. Furthermore, this and other intelligent inks and ink elements may have a shelf life of several months or more, or may be stabilized with preservatives such as antioxidants to ensure a shelf life of several months or more. The tattoo itself (and the intelligent ink thereof) may be stable for years after injection into the skin. Additional ink elements at or in the same injection site as the tattoo may extend the life of the tattoo even further, such as UV blocking agents to reduce degradation due to sun exposure.

In some embodiments, intelligent inks may respond to antigens or other markers of an animal. For example, the intelligent ink may include cyclodextrin functionalized with a dye chromophore and modified to preferentially accept a target hormone. The dye chromophore may include methyl red, the color of which may be suppressed by cyclodextrin unless the target hormone is present within the cyclodextrin as a guest where the dye chromophore may appear orange under subcutaneous pH. This intelligent ink may not amplify the hormone signal, and may not be highly sensitive. As such, this intelligent ink may be used to detect incremental cumulative increases in hormone levels, rather than nanomolar thresholds. For example, this intelligent ink may be used to detect incremental cumulative increases in a micromolar to millimolar range.

In some embodiments, another intelligent ink may include a surface antibody or aptamer functionalized gold nanoparticles in polymer encapsulated solution. These particles may create a strong structural color when individually suspended. This structural color may be altered, generally from red to blue, when a corresponding target molecule binds to the surface antibody and causes agglomeration of the nanoparticles. This intelligent ink may have a magnifying effect, as one binding target molecule can agglomerate several nanoparticles. Gold nanoparticle surface aptamers for cortisol and similar molecules exist and can be used in the intelligent ink to detect cortisol or other similar molecules. This intelligent ink can detect concentrations as low as 40 ng/mL, 30 ng/mL, or 20 ng/mL for cortisol, for example. High sensitivity combined with low level amplification may lead to good performance of this intelligent ink as a threshold test.

In some embodiments, another intelligent ink may include an encapsulation of a hormone antibody conjugated to alkaline phosphatase. This conjugation may inhibit an enzyme function. The encapsulation may include a standard phosphatase stain e.g. Fast red violet—Napthol AS-BI phosphate, which may remain inactivated until hormone-antibody binding occurs. Phosphatase activity may be restored in response to the hormone-antibody binding and the stain may be activated.

In some embodiments, another intelligent ink may include a bacterial cell line that may be a simple and controllable hormone responsive line that may have both a tunable amplification response and a controllable expression. For example, E. coli strains can be engineered to respond to many target molecules, and to amplify the signal in a tunable manner. Natural bacterial response to steroids exists, and can be genetically transferred to E. Coli via plasmid. This response may occur down to at least 0.5 millimolar (mM), or down to a nanomolar (nM) range or a picomolar (pM) range. A simple form of color change may include expression of bleaching enzymes into an enzyme sensitive dye filled encapsulant such as Remazol Black-B.

In some embodiments, another intelligent ink may include an immortalized chromatophore or melanophore cell line from fish, such as Betta splendins, to sense and provide a color or shade response to specific hormones. The target specificity may be produced by modification of existing responses within the cell as melanophores respond to steroid concentrations. These cells may be encapsulated where they are still capable of receiving blood solutes.

Another intelligent ink may include encapsulated tissue engineered from animal chromatophore organ to be responsive to a target hormone, similar to the operation of cellular level modified melanophores. Squid, octopus and some bony fish possess organ tissue that can be used for this purpose. The tissue may be well suited to propagation and modification.

In some embodiments, another intelligent ink may include particles possessing a 1-2-, or 3-dimensional photonic crystal structure, such as inverse opal, constructed from molecularly imprinted polymer (MIP) with target hormone imprints. The finely tuned photonic structure may produce a specific structural color, dependent on spacing of porous elements. The MIP may take up the target molecule into its imprints and undergo a process of expansion, which may cause a change in spacing and thus a visible change in the structural color of the component. MIP uptake of progesterone and cortisol have been developed. The components can detect analyte concentrations below 50 ng/L.

In some embodiments, another intelligent ink may include particles that include an encapsulated orientation controlled liquid crystal film over hormone antibodies immobilized on a permeable polymer element that allows hormone access to the hormone antibodies. The liquid crystals may be vertically aligned when hormone antibodies are unbound to the target antigen and thus appear dark. When antigen binding occurs, the liquid crystals may rotate and become highly reflective of polarized light.

Some or all of the intelligent inks described above may be responsive to target hormones. Various intelligent inks will now be described that may be responsive to one or more other targets of physiology, such as temperature. Some intelligent inks that are responsive to temperature may include thermochromic leuco dyes that reversibly or irreversibly bleach or color over a temperature range. An example leuco dye suitable for use as an intelligent ink may include crystal violet lactone with temperature controllable protonation. The leuco dye may be encapsulated in a microcapsule with an organic acid salt and a solvent selected for a specific melting point (m.p.) to achieve about a 39° C. transition point (or other suitable transition point) for the color/bleaching change of the leuco dye. As a particular example, the leuco dye may be encapsulated with, e.g., fatty alcohol mixtures such as myristyl alcohol (m.p. 38° C.) with a low percentage of cetyl alcohol (m.p. 49° C.). The 39° C. transition point in this example may be suitable for use in cattle or other animals with a standard body temperature in adult cattle of about 38-38.5° C. In this example, in response to melting of the solvent, the solutes may become mobile and the pH may drop, which may produce a structural change in the leuco dye that causes a color change within the microcapsule. The microcapsule may be referred to as a sensor.

In some embodiments, encapsulation of such sensors may be performed within polyester or polystyrene spheres on the order of 3-5 micrometers (μm), or within other suitable inert polymer microcapsules.

In some embodiments, the chemistry of the intelligent ink that includes the encapsulated leuco dye may provide a reversible change. Some leuco dyes are available that may undergo irreversible color changes and may provide permanent recording of temperature spikes. A variety of these leuco dyes are available off the shelf.

In some embodiments, intelligent inks that include leuco dyes that undergo reversible or irreversible color changes may be visible to the naked eye and/or may be measurable by a tattoo reader. The color change may include a change from a colorless and/or natural light skin tone to a dark intense violet color.

In some embodiments, the concentrations of 1-250 mg, or 100-250 mg, or 200-250 mg of microcapsules per square cm of skin may be injected to produce a highly visible marker. The microcapsules may be delivered suspended in a skin absorbed carrier such as isopropyl myristate. A size of the area being tattooed and a concentration of the intelligent ink in the area being tattooed may be adjusted, as needed.

In this example, the active components (e.g., the leuco dye) in the microcapsules may be stable and long-lived, and may be encapsulated within an inert polymer such as a cellulose derivative, which may prevent access of external agents. Accordingly, this intelligent ink may be capable of sensing relevant temperature changes within the skin of an animal for 2-3 years or for some other period of time.

In some embodiments, the intelligent ink can be tailored to respond to relevant temperatures at a desired location by altering the composition balance of the encapsulated solvents. At a suitable depth in the skin and placement on the body of the animal the intelligent ink may have access to body heat for which its temperature responsive range can be calibrated. Depending on the location of the tattoo on the body, a core body temperature may be inferred from a previously established relationship between skin temperature and core temperature.

In some embodiments, the intelligent ink may be injected to a relatively shallow depth in the skin to detect skin temperature, which may be influenced by ambient temperature. In other embodiments, the intelligent ink may be injected to a relatively deeper depth, e.g., into a fat layer in the subcutis or below to detect core temperature and/or to reduce an influence of the ambient temperature on the intelligent ink.

The leuco dyes contained within the microcapsules may undergo reversible changes without being damaged by cycling from colored to colorless below about 60° C.

In some embodiments, intelligent ink may be placed anywhere within the dermis, which may include a depth of between 0.1 mm to 4 mm deep depending on the breed and species of the animal, or at any other layer or depth in the skin of the animal. The depth may vary if used in different animals or different sites on an animal. The significant darkening color change when the temperature of the injection site exceeds the transition point of the intelligent ink may make this intelligent ink strongly visible through the skin.

In some embodiments, no further amplification may be needed to perceive (e.g., by the naked eye or using a tattoo reader) the change in the intelligent ink as the temperature change may affect all microcapsules individually.

In some embodiments, The capsules may be inert, may contain no toxic components, and may be delivered only to the skin. These types of microcapsules have been employed in clothing and indicators on food and beverage containers. They may not present a health risk to animals or people, and any consumption or intake may be extremely low due to the placement and large size of the microcapsules. In some embodiments the capsules may be reactive, and form a part of the intelligent ink interaction with the animal body.

In some embodiments, the shelf life of this intelligent ink may be greater than 5 years under correct storage conditions, e.g., standard room temperature of about 25° C. and no direct sunlight.

In some embodiments, the intelligent inks other than those described above may respond to temperature or other target markers. For example, an intelligent ink may include a hygroscopic anhydrous salt that gains or changes color upon hydration. The hygroscopic anhydrous salt may be encapsulated within a microcapsule of a polymer with side chains that crystallize at a different temperature, referred to as a transition point, to that of backbone chains. The transition point may be finely tunable. When the side chains are crystallized (e.g., at temperatures below the transition point) the polymer's water permeability may be extremely low. At temperatures above the transition point, the side chains may relax and allow passage of water vapor. The hygroscopic anhydrous salt, which may be colorless when not exposed to moisture, may be exposed to moisture when the side chains relax and allow passage of water vapor, thereby becoming colored. The hygroscopic anhydrous salts may include cobalt chloride, copper sulphate, or other hygroscopic anhydrous salt. The color change in this intelligent ink may be irreversible.

In some embodiments, the intelligent ink may include encapsulated thermochromic cholesteric or chiral nematic liquid crystal oils that are delivered into the cutis. The liquid crystal oils may be encapsulated by a number of techniques including coacervation with a compatible polymer. Cholesteric liquid crystals may be precisely tunable to specific transition temperatures, and transition ranges, and may be tuned to transition visibly and accurately from red to blue between 37° C. to 40° C. or some other temperature range. This intelligent ink may be used for continual temperature monitoring of animals, as a single piece of material may be capable of producing a range of colors for a temperature range, allowing identification of low, moderate and high temperatures and intermediates.

In some embodiments, intelligent ink may include PDA polymers that undergo a color change from blue to red under rising temperatures between 23° C. to 130° C. A specific region of this range can be selected by altering an initial monomer for polymerization. This intelligent ink may be used to produce reversible or irreversible changes.

In some embodiments, intelligent ink may include Astaxanthin bound to another protein. Astaxanthin is a brightly orange/red colored carotenoid when in a free state (e.g., when it is not bound to another protein). The color of Astaxanthin may be suppressed when not in the free state (e.g., when it is bound to another protein). The color may return after the bound protein has been denatured. Astaxanthin has a high denaturation temperature well above that achievable within the body of an animal, and thus cannot be thermally denatured within the body of the animal. However Astaxanthin can be bound to proteins that begin denaturing at ˜40° C., which is a property of many intracellular proteins, thus acting as a biologically relevant thermochromic indicator. The color change in this intelligent ink may be irreversible.

In some embodiments, intelligent ink may include a compound of copper (Cu) nitrogen dioxide (NO2)2 ammonia (NH₃)₂, which is a copper based compound that converts from green to purple at 35° C. The intelligent ink may also include Bis (N,N-diethylethylenediamine)copper(II)perchlorate, which reversibly changes color from red to deep blue-purple at 43° C. The combination of CU(NO₂)₂(NH₃)₂ and Bis (N,N-diethylethylenediamine)copper(II)perchlorate establishes a temperature window of 35° C. to 43° C., the departure from which may be visible by dramatic color change. These compounds may be robustly encapsulated in polymer microspheres to avoid chemical interaction with the in-animal environment.

Various embodiments of intelligent inks will now be described that may be responsive to one or more targets of physiology other than target hormones or temperature. For example, the intelligent ink may include a color changing marker for glucose that can be injected into the skin of the animal. The color changing marker for glucose may have good correlation to blood glucose measurements taken using a glucometer. One such marker or intelligent ink in an animal tattoo may operate as follows. The intelligent ink of the tattoo may contain nanospheres that contain a covalently bound phenylboronic acid derivative as well as two attached fluorophores that have been synthesized. The nanospheres may vary in size dependent on sugar concentration. In the absence of glucose the nanospheres may be small and may thereby provide a relatively dense or concentrated color for the tattoo. As glucose concentration increases, the glucose may bond with the acid and may increase the size of the fluorophores, which may in turn decrease the color density or color concentration of the tattoo, or at least of the part of the tattoo that includes the intelligent ink.

Another such marker or intelligent ink may target hydration level of the animal by responding to electrolytes in the blood or elsewhere in the animal. For example, a responsive dye used in the intelligent ink may be encapsulated within microspheres, such as 120 micron microspheres. The microspheres may be coated with a biocompatible material and may be implanted in the subcutis of the animal as the intelligent ink that makes up all or a part of the tattoo. The microspheres may be configured to be porous to small cations. Cations from interstitial space may migrate into the microspheres such that the concentration within the microspheres and within interstitial space may be the same. The presence of cations near the dye may cause the dye to lose an electron and become fluorescent. The relative fluorescence of the portion of the tattoo that includes the intelligent ink made up of these microspheres can be used to determine sodium concentration within interstitial space, from which hydration level can be inferred.

Other intelligent inks may alternately or additionally be implemented for animal lifecycle monitoring as described herein.

Various use cases may exist for the tattoos with intelligent inks described herein. In one such case, one or more cattle in a herd of cattle may be tattooed with one or more intelligent inks to monitor the physiological status of the tattooed animals in the herd. A farmer, farmhand, rancher, ranch hand, veterinarian, or other person may be with the animals in a remote location, e.g., on a cattle drive, with little or no access to any communication networks or computing devices, or in a location in which it may be inconvenient to access communication networks or computing devices. The person may observe the tattoo of each of the tattooed animals with the naked eye, determine a physiological status of the animal based on the tattoo, and then take some other action. Actions the person may take may include treating a physiological event or condition indicated by the determined physiological status, separating the animal from other animals that do not have the same physiological event or condition, or some other action. For example, if the tattoo of an animal indicates the animal is pregnant, the animal may be treated for pregnancy. If the tattoo of the animal indicates the animal has a pathogen, the animal may be separated or quarantined from other animals whose tattoos indicate they do not have the pathogen. If the tattoo of the animal indicates the animal's blood glucose levels are relatively high, which may indicate the animal is consuming muscle or liver glycogen and is likely to be dark cutting, the animal may be separated from a group of animals being sent to slaughter.

In another potential use case, tattoos with intelligent inks may be used in conservation efforts for, e.g., endangered species or other species. Examples include some species of rhinoceros, panda, and tigers and there are many others. In an example implementation, breeding females within an endangered species may be tattooed with an intelligent ink that responds to hormones associated with estrus in the species. When the tattoo of a breeding female indicates the breeding female is in estrus, arrangements may be made to unite the breeding female with one or more breeding males of the species during some or all of the breeding female's estrus phase.

In another potential use case, tattoos with intelligent ink may be applied to pets, such as dogs, cats, ferrets, turtles, snakes, or other pets. In this example, the intelligent ink may be responsive to one or more pathogens, hormones associated with estrus and/or pregnancy, target markers associated with breed-specific physiological events or conditions, or other target markers. Owners, breeders, veterinarians, and/or other individuals associated with the pets may observe the tattoos of their pets to monitor whether their pets are infected, in estrus, pregnant, or have some other physiological event or condition. The individuals may take their pets to the veterinarian, isolate them from other members of the same species during estrus to avoid unwanted pregnancy, hire a stud or artificially inseminate their pets during estrus to initiate pregnancy, modify the diet or daily exercise of their pet while their pet is pregnant, or take some other action in response to the tattoos indicating that their pets are infected with a pathogen, in estrus, pregnant, or have some other physiological event or condition.

Other potential use cases involve humans, which fall under the broad umbrella of the term animal. As an example of a use case that involves humans, military personnel or other humans may be tattooed with one or more intelligent inks that are responsive to hormones associated with stress or depression and/or to other target markers. The military personnel and/or their superiors or other individuals may monitor the tattoos of the military personnel to determine their battle readiness or other physiological status and/or psychological status of the military personnel. If the tattoos indicate the military personnel are stressed or depressed, one or more stress-reduction or depression treatments may be recommended or prescribed for the military personnel or some other action may be taken.

In another embodiment, tattoos with intelligent inks may be used to monitor oxytocin levels and/or edema in healthcare settings or other settings. For example, an expecting mother may be tattooed with an intelligent ink that is responsive to the expecting mother's oxytocin level. If the tattoo indicates the expecting mother's oxytocin level is high and/or climbing and the expecting mother is not already with a healthcare provider for childbirth, the expecting mother may make appropriate arrangements, e.g., by driving to a hospital or contacting a midwife, nurse, doctor, or other healthcare provider that makes house calls to come attend to the expecting mother. Alternatively or additionally, if the tattoo indicates the expecting mother's oxytocin level is low and her baby is at or near full term, an intravenous infusion of oxytocin may be administered to the expecting mother to induce labor. Alternatively or additionally, after delivery of the baby, if the tattoo indicates the mother's oxytocin level is low and/or the mother is experiencing difficulty producing milk, oxytocin may be administered to the mother to stimulate milk release. These or other actions may be taken responsive to an oxytocin level indicated by the tattoo.

In another embodiment, patients in a hospital or other settings may be tattooed with an intelligent ink that is responsive to one or more target markers (e.g., fluid located in the interstitium) associated with edema. If the tattoo indicates onset of edema in a patient, appropriate treatment may be administered to the patient.

In still another embodiment, humans with diabetes (diabetics) may be tattooed with one or more intelligent inks that respond to blood glucose levels. Many diabetics test their blood glucose levels multiple times a day by pricking a finger or other location on their body to draw blood, applying some of the blood to a test strip, and inserting the test strip into a blood glucose meter to determine the diabetic's blood glucose level. A diabetic with a tattoo with one or more intelligent inks that respond to blood glucose levels may observe the tattoo to determine his or her blood glucose levels in a non-invasive manner, e.g., without pricking himself or herself to draw blood. For example, the intelligent ink may be configured to turn one color when the blood glucose level is below a threshold level and another color when the blood glucose level is above the threshold level. Alternatively or additionally, the intelligent ink may be configured to gradually transition from one color to the other over a range of blood glucose level values, with the color of the intelligent ink at a given moment being indicative of a particular blood glucose level within the range. Alternatively or additionally, the diabetic may use an optical imager to measure the color or other optical property of the intelligent ink to infer the diabetic's blood glucose level at the moment. If the tattoo indicates the diabetic's blood glucose level is relatively high, the diabetic may administer insulin to him or herself to lower the diabetic's blood glucose level. If the tattoo indicates the diabetic's blood glucose level is relatively low, the diabetic may consume some candy, juice, or other food or drink to raise the diabetic's blood glucose level.

In another embodiment, humans experiencing fertility problems may be tattooed with one or more intelligent inks that respond to hormones associated with ovulation and/or pregnancy. When the tattoo indicates that a woman is ovulating, she may take appropriate action to initiate pregnancy. Alternatively or additionally, when the tattoo indicates that the woman is pregnant, she may modify her diet, exercise, work schedule, and/or other aspects of her life to reduce a likelihood of miscarriage.

In another embodiment, humans may tattoo themselves with intelligent inks for purely aesthetic reasons. For instance, some humans may want a tattoo that includes one or more intelligent inks that vary in color or some other optical property responsive to one or more target markers of the humans.

Some potential use cases may involve one or more of a tattoo reader, a tattoo applicator, a server, a computing device, and/or a network. The tattoo application may be manually or automatically operated to tattoo the animal with an intelligent ink.

The tattoo reader may read the tattoo to determine a physiological condition of the animal. In these and other examples, the tattoo reader may include an optical imager and/or a radiation source. The radiation source may emit white light, UV light, or other light to irradiate the tattoo. The optical imager may measure one or more variable optical properties of each intelligent ink and/or of one or more non-intelligent inks included in the tattoo to read the tattoo. The optical properties may include a color of the corresponding ink, or more particularly a wavelength or wavelengths of light reflected by the corresponding ink, color saturation of the corresponding ink, fluorescence of the corresponding ink, or other optical property of the corresponding ink. The optical property may be measured by measuring the optical property of light reflected, transmitted, emitted, and/or interfered by the corresponding ink. The measured optical property may be provided to the server and/or may be retained at the computing device which may include or be coupled to the tattoo reader.

The server or the computing device may include one or more animal records and/or a health monitor application. Each animal record may include a record of each animal with a tattoo. Each of the animal records may include physiological status generated and/or recorded by the health monitor application for a corresponding one of the animals. The physiological status may include one-time or repeated measurements of the optical property of each intelligent ink and/or non-intelligent ink included in the tattoo of the corresponding animal. Alternately or additionally, the physiological status may include interpretations of measurements of the optical property. For example, the measurements of the optical property may include numerical values of the optical property, each numerical value corresponding to a different measurement. In comparison, each interpretation may interpret the corresponding numerical value as indicating that a particular target of physiology is normal or abnormal, is within or without a particular range, is indicative (or not) of a particular physiological event or condition, or may provide some other interpretation of the numerical values. In some embodiments, entries of measurements and/or interpretations of measurements in each of the animal records may each include a timestamp or another indicator of when the measurements are received from the tattoo reader and/or entered into the animal records.

The tattoo reader, tattoo applicator, server, computing device, and/or the network may be involved in a method to monitor physiological status of animals. The method may include forming a tattoo with intelligent ink in skin of an animal, e.g., using the tattoo applicator. The method may also include determining a physiological status of the animal based on the tattoo. The method may also include recording the physiological status and determining if the physiological status indicates a physiological event or condition. If the physiological status indicates the physiological event or condition, an alert may be triggered to address the physiological event or condition. The method may additionally include addressing the physiological event or condition in response to the trigger.

As can be seen from the above examples, the technology described herein is applicable to any kind of animal that can accept this technology. The above are non-limiting examples, and virtually any animal may be a potential receiver of this technology, including land animals, marine animals, birds, worms, amphibians, reptiles, and other animals.

Tattoos as described herein may be applied using any suitable tattoo applicator, a variety of which are described herein. Some of the tattoo applicators described herein include needles or microneedles of a same length and/or that penetrate to a same layer or depth of the skin. In these embodiments and other embodiments, one or more ink elements may be injected to the same layer or depth of the skin.

In some embodiments, tattoo applicators described herein include needles or microneedles of different lengths and/or that penetrate to different layers or depths of the skin. In these and other embodiments, one or more ink elements may be injected to one layer or depth of the skin, while one or more other ink elements may be injected to another layer or depth of the skin. As a particular example, a tattoo applicator may include some microneedles of one length that inject a depigmentation agent into epidermis of the skin, other microneedles of another length that inject an intelligent ink into dermis of the skin, and still other microneedles of yet another length that inject a depilatory agent into a lower depth in the dermis and/or into subcutis of the skin. In some embodiments, the different ink elements may be injected to different layers or depths at the same time without separately injecting needles or microneedles of one length at different times then needles or microneedles of a different length. Alternatively or additionally, some needles or microneedles may deliver multiple ink elements per needle or per microneedle to different layers or depths of the skin.

In addition to needles or microneedles, some embodiments of one or more tattoo applicator(s) may include one or more of an ink delivery mechanism, an ink reservoir, a sanitation mechanism, a cleansing mechanism, and a pattern selection mechanism. The ink delivery mechanism may be configured to deliver ink to the needles or microneedles such that the needles or microneedles may inject the ink into a corresponding layer or depth in the skin. For example, the ink delivery mechanism may include a roller that is configured to roll ink onto a surface of the skin, which may then be injected into the skin by the needles or microneedles. The ink roller may include multiple areas, each with a different ink or ink element. As another example, the ink delivery mechanism may include an ink pad that the needles or microneedles pass through before penetrating the skin; the needles or microneedles may retain some of the ink on their tips as they pass through the ink pad and may inject the retained ink into the skin. The ink pad may include multiple areas, each with a different ink or ink element. As another example, where the needles or microneedles include perforated needles, the ink delivery mechanism may include a pump, tubing, and/or or other apparatus or device to deliver ink through hollow portions of the perforated needles into the skin. In this example, the ink delivery mechanism may deliver different inks or ink elements to different perforated needles for injection into the skin.

In some embodiments, the ink reservoir may be configured to retain ink and to supply ink to the ink delivery mechanism and/or to the needles or microneedles. The ink reservoir may be partitioned to retain different inks and/or ink elements.

In some embodiments, the applicator may include a sanitation mechanism. The sanitation mechanism may be configured to apply a sanitation agent to the needles or microneedles between applications of tattoos to different animals. For example, the sanitation mechanism may include a sponge saturated in the sanitation agent. The needles or microneedles may be retracted into the sponge when not penetrating the skin such that the needles or microneedles are in contact with the sanitation agent. Alternatively or additionally, where the needles or microneedles are perforated needles, the sanitation mechanism may be configured to route the sanitation agent through the hollow portion of the perforated needles.

In some embodiments, the applicator may include a cleansing mechanism. The cleansing mechanism may be configured to cleanse the injection site of the skin to which a tattoo is to be applied. The cleansing mechanism may include a sponge that is saturated in a cleansing agent.

In some embodiments, the applicator may include a pattern selection mechanism. The pattern selection mechanism may be configured to change an arrangement or pattern of the needles or microneedles. The pattern selection mechanism may include one or more selectors, each with multiple different patterns of needles or microneedles. Each selector may be operated to select one of the multiple different patterns of the selector.

In various embodiments, the tattoo applicators may include microneedles made of self-dissolving, skin-absorbable materials. Each of the microneedles may include one or more ink elements infused or otherwise contained therein. When a tattoo is formed using tattoo applicators with these microneedles, the microneedles may penetrate the skin and may be left in the skin to dissolve and release the ink elements contained therein into the skin.

In an embodiment, a tattoo applicator may include a substrate with multiple microneedles arranged on the substrate. The microneedles may include self-dissolving, skin-absorbable materials. The substrate may be configured to peel away or break away from the microneedles after injection to leave the microneedles in the skin. A tattoo may be formed by such a tattoo applicator by injecting the microneedles into the skin and peeling the substrate away from the microneedles. The microneedles may then release one or more ink elements contained therein into the skin.

In some embodiments, the microneedles of a tattoo applicator may have different lengths. For example, some of the microneedles may have one length, others may have another length, and still others may have still another length. The microneedles of different lengths may include different ink elements. For example, the microneedles of one length may include one ink element, the microneedles of another length may include another ink element, and the microneedles of still another length may include still another ink element.

Alternatively or additionally, in some embodiments, the microneedles of the tattoo applicator may include multiple ink elements per microneedle at the same or different depths of the microneedle. For example, each of these microneedles may include one ink element in a tip of the microneedle, another ink element in a middle of the microneedle, and still another ink element in a base of the microneedle.

In another embodiment, the tattoo applicator may include a branding iron configuration. In particular, the tattoo applicator may include a handle and a plate. The tattoo applicator may additionally include one or more substrates with peel and stick backing on one side and microneedles on an opposite side. The peel and stick backing may be removed from the substrate to attach the substrate to the plate, and the handle may be moved, as desired, to position the substrate at a desired injection site of an animal. The handle may be further moved to push the substrate with microneedles toward the skin at the injection site and to inject the microneedles into the skin. The handle may also be moved to peel the substrate away from the injected microneedles. The substrate without the microneedles may be removed from the plate and the process may be repeated with a new substrate with microneedles to form a new tattoo on the same or a different animal.

Reference will now be made to the drawings to describe various aspects of some example embodiments. It is to be understood that the drawings are diagrammatic and schematic representations of such example embodiments, and are not limiting of the present invention, nor are they necessarily drawn to scale.

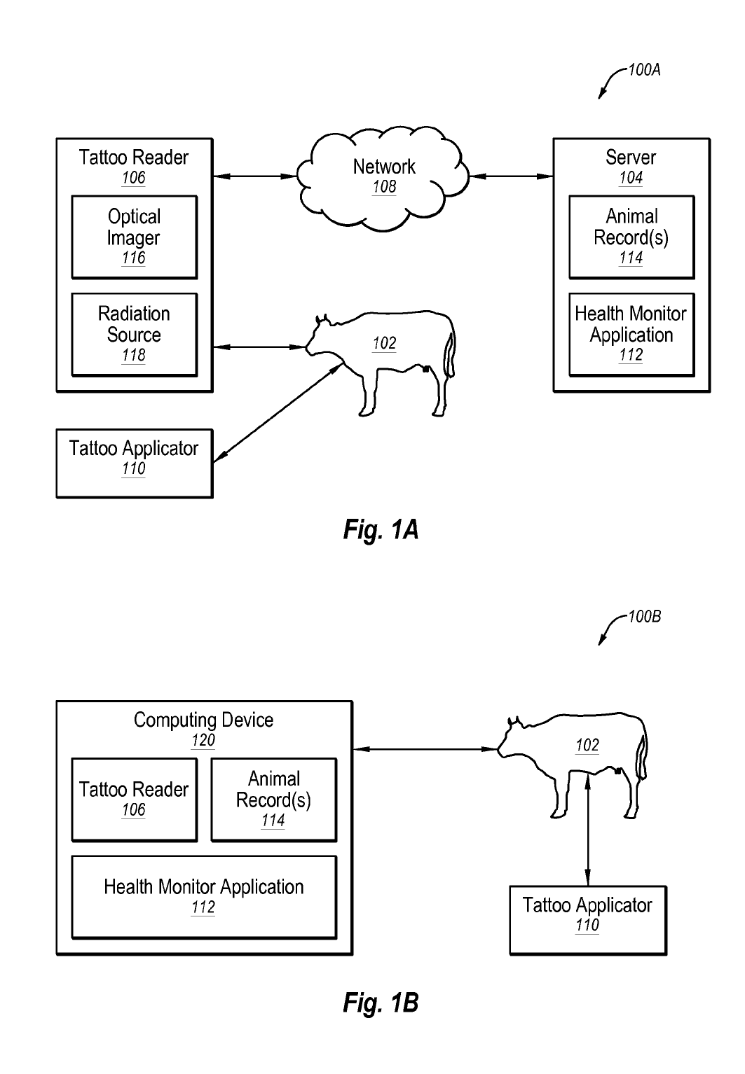

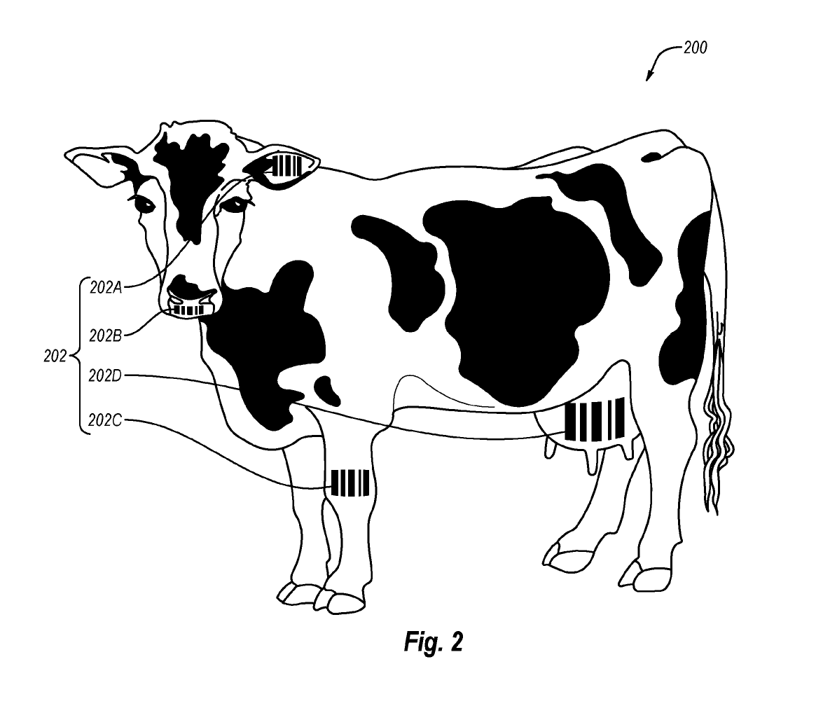

FIG. 1A is a diagram of an example system 100A to monitor one or more animals 102 (hereinafter “animal 102” or “animals 102”), arranged in accordance with at least some embodiments described herein. The system 100A may, in general, monitor a lifecycle and/or health of the animals 102 based on one or more tattoos (see FIG. 2) applied to the animals 102. The tattoos applied to the animals 102 may each include one or more intelligent inks and/or other ink elements. Alternately or additionally, the tattoos applied to the animals 102 may include or indicate a unique identifier of each of the animals 102 and/or other information.

The system 100A may include a server 104, a tattoo reader 106, and a network 108. Alternately or additionally, the system 100A may include a tattoo applicator 110. In general, the network 108 may include one or more wide area networks (WANs) and/or local area networks (LANs) that enable the server 104, the tattoo reader 106, and/or the tattoo applicator 110 to communicate with each other. In some embodiments, the network 108 includes the Internet, including a global internetwork formed by logical and physical connections between multiple WANs and/or LANs. Alternately or additionally, the network 108 may include one or more cellular RF networks and/or one or more wired and/or wireless networks such as, 802.xx networks, Bluetooth access points, wireless access points, IP-based networks, or the like. The network 108 may also include servers that enable one type of network to interface with another type of network.

The server 104 may in general be configured to host a health monitor application 112 and may include one or more animal records 114. In these and other embodiments, the server 104 may include computing resources such as a processor and a computer-readable medium communicatively coupled to the processor. The health monitor application 112 may be at least temporarily loaded into memory or other volatile storage of the server 104 for execution by the processor. Alternately or additionally, the health monitor application 112 may be executed by the processor line by line from a hard disk or other non-volatile storage of the server 104. The memory and hard disk are both examples of the computer-readable medium, and others are described below with respect to FIG. 11. The health monitor application 112 may generally be configured to perform one or more of the operations described herein. In particular, the health monitor application 112 may include computer instructions that, in response to execution by the processor, cause the processor to perform or control performance of one or more of the operations described herein.

The animal records 114 may be stored on the server 104, as illustrated in FIG. 1A. For example, the animal records 114 may be stored in the computer-readable medium of the server 104. Alternately or additionally, the animal records 114 may be stored remotely in cloud storage and/or in a computer-readable medium that is remote from and communicatively coupled to the server 104. Alternately or additionally, the animal records 114 may be stored in hardcopy form, such as in a notebook, journal, diary, or physical file system.

The animal records 114 may include a record for each of the animals 102. Each of the animal records 114 may include physiological status generated and/or recorded by the health monitor application 112 for the corresponding one of the animals 102. The physiological status may include one-time or repeated measurements of an optical property of the intelligent ink and/or other ink elements included in the tattoos of the animals 102. The optical property of the intelligent ink may include a variable optical property. Alternately or additionally, the physiological status may include interpretations of measurements of the optical property. For example, the measurements of the optical property may include numerical values of the optical property, each numerical value corresponding to a different measurement. In comparison, each interpretation may interpret the corresponding numerical value as indicating that a particular target of physiology is normal or abnormal, is within or without a particular range, is indicative (or not) of a particular physiological event or condition, or may provide some other interpretation of the numerical values. In some embodiments, entries of measurements and/or interpretations of measurements in each of the animal records 114 may each include a timestamp or another indicator of when the measurements are received from the tattoo reader and/or entered into the animal records 114.

The particular target of physiology of the animal 102 to which each intelligent ink is configured to respond may include a glucose level of the animal 102, a hydration level of the animal 102, a temperature of the animal 102, a pH level of the animal 102, a level of a particular hormone in the animal 102, a particular pathogen in the animal 102, a particular antigen in the animal 102, or other target of physiology. Alternately or additionally, the particular target of physiology of the animal 102 may include salts, minerals, vitamins, sugars, carbohydrates, lipids, phospholipids, proteins, nucleic acids, polynucleotides, antibodies, immunoregulatory molecules, disease markers, or other evidence of a physiological condition or event in the animal 102. Accordingly, the particular target of physiology of the animal 102 may include blood markers, interstitial markers, or other markers indicative of a physiological condition or event.

In some embodiments, each of at least some of the animals 102 may include a tattoo with multiple different intelligent inks In these and other embodiments, the health records 114 for the animals 102 with tattoos with multiple different intelligent inks may include one-time or repeated measurements of at least one optical property for one or more, up to all, of the different intelligent inks. The measured optical property may be the same or different for the multiple different intelligent inks. For example, color saturation may be measured for one of the different intelligent inks while color saturation, fluorescence, color, or another optical property may be measured for another of the different intelligent inks. Alternately or additionally, the health records 114 may include interpretations of one or more of the one-time or repeated measurements.

Each tattoo may additionally include one or more non-intelligent inks. A non-intelligent ink may include a tattoo ink that does not respond to or depend on the physiology of the animals 102. The non-intelligent ink may be used as a static reference and/or may provide a baseline against which the intelligent ink is compared. For example, the optical property of the intelligent ink may be measured, the same optical property of the non-intelligent ink may also be measured, and the two measurements may be compared to each other. The optical property of the non-intelligent ink may be equal or substantially equal to the same optical property of the intelligent ink prior to (or after) the intelligent ink responds to the target of physiology. Alternately or additionally, whether or not the optical property of the non-intelligent ink is ever equal or substantially equal to the same optical property of the intelligent ink, the optical property of the non-intelligent ink may provide a static baseline. In these and other embodiments, a difference between the measurements of the optical property of the non-intelligent ink and of the intelligent ink may indicate a level of the target of physiology. In some embodiments, the animal records 114 may include measurements of the non-intelligent ink, calculations of the difference between the measurements of the non-intelligent ink and of the intelligent ink, interpretations of any of the foregoing, and/or corresponding timestamps. Alternately or additionally, the intelligent ink and/or the tattoo that includes the intelligent ink may convey or include information such as a nature of the tattoo (e.g., the target to which it is configured to respond), a brand, test meanings, and/or other information.

The tattoo reader 106 may be configured to read the tattoos of the animals 102. In some embodiments, the tattoo reader 106 may be configured to read the tattoos by measuring one or more optical properties of each of the intelligent inks and/or non-intelligent inks included in the tattoos of the animals 102. Accordingly, the tattoo reader 106 may include an optical imager 116. The optical imager 116 may include a camera, a charge-coupled device (CCD), an active-pixel sensor (APS), a complementary metal-oxide-semiconductor (CMOS) device, a photodiode, or other suitable optical imager.

The optical imager 116 may be configured to measure one or more optical properties of each of the intelligent inks and/or the non-intelligent inks. The optical properties may include a color of the corresponding ink, or more particularly a wavelength or wavelengths of light reflected by the corresponding ink, color saturation of the corresponding ink, fluorescence of the corresponding ink, or other optical property of the corresponding ink. The optical property may be measured by measuring the optical property of light reflected, transmitted, emitted, and/or interfered by the corresponding ink. The term “light” as used herein may generally include electromagnetic radiation of any wavelength, including those within the visible spectrum as well as the x-ray spectrum, the microwave spectrum, the ultraviolet (UV) spectrum, the infrared (IR) spectrum, and/or other electromagnetic waves. The optical imager 116 may measure the one or more optical properties by receiving light reflected, transmitted, emitted, and/or interfered by the corresponding ink and generating an electric signal therefrom that represents a value of the optical property being measured.

Alternately or additionally, the optical imager 116 may be configured to capture an image of at least a portion of the tattoos. In some embodiments, the optical imager 116 may include a visible light spectrum imager that captures light within the visible light spectrum, which may include a wavelength range from about 380 nanometers (nm) to about 800 nm. Alternately or additionally, the imager 130 may be configured to capture light from the x-ray portion of the electromagnetic spectrum, the UV portion of the electromagnetic spectrum, the IR portion of the electromagnetic spectrum, among potentially other portions of the electromagnetic spectrum. The optical imager 116 may generate electrical signals and/or data that may represent one or more optical properties of the intelligent ink in the tattoos of the animals 102 based on the capture of light reflected, transmitted, emitted, and/or interfered by the intelligent ink.

In some embodiments, the tattoo reader 106 may additionally include a radiation source 118. The radiation source 118 may be configured to emit electromagnetic radiation that may be directed toward the tattoos of the animals 102. The radiation source 118 may include a white light source, a UV light source, or another suitable light source. The radiation may illuminate the tattoos to allow the tattoos to be read or imaged, and at least a portion of the wavelength spectrum of the radiation may be reflected by the inks of the tattoos back to the optical imager 116 as the light that is received by the optical imager 116. Alternately or additionally, at least some of the wavelength spectrum of the radiation may be absorbed by at least one of the intelligent inks of the tattoos such that the at least one of the intelligent inks subsequently emits fluorescence that may be received and read by the optical imager 116.

The tattoo applicator 110 may include any system, apparatus, or device to apply a tattoo to the animals 102. The tattoo applicator 110 may include an automated tattoo applicator, a semi-automated tattoo applicator, or a manual tattoo applicator. In some embodiments in which the tattoo applicator 110 is an automated or semi-automated tattoo applicator, the tattoo applicator 110 may be communicatively coupled to the server 104 and/or the tattoo reader 106 through the network 108 or other suitable connection and/or may be automatically or semi-automatically controlled by the server 104 and/or the tattoo reader 106 to automatically or semi-automatically apply tattoos to the animals 102.

In an example embodiment, the tattoo applicator 110 includes a plier-type tattoo applicator with opposing jaws, which may be used to apply tattoos to ears of the animals 102 or other locations on the animals 102. One or more tattoo needles may be secured on one of the jaws facing inward toward the other one of the jaws. After application of one or more intelligent and/or non-intelligent inks or other tattoo ink elements to an injection site on the skin of the animal 102, e.g., inside an ear of the animal, and placement of the ear between the jaws of the tattoo applicator 110 with the tattoo needles arranged to come in contact with the injection site to which the ink is applied, handles of the tattoo applicator 110 may be squeezed to cause the jaws of the tattoo applicator 110 to come together and squeeze the ear. As the ear is squeezed between the jaws, the tattoo needles may penetrate the skin of the animal and force the ink from the surface of the skin inward to a particular depth, such as into epidermis stratum basale, dermis, or subcutis of the skin, or other layer of the skin.

In another embodiment, the tattoo applicator 110 is based on microneedle vaccine technology. For example, the tattoo applicator 110 may include a patch with multiple microneedles, such as a thousand microneedles or more. The patch may be placed on the skin with the microneedles penetrating a particular depth into the skin, or multiple depths into the skin. The microneedles may include therein reservoirs of ink and/or may be hollow and may be in fluid communication with a reservoir of ink elsewhere in the patch. After the patch has been applied with the microneedles penetrating the skin, the microneedles may dissolve to release the ink contained therein into the skin and/or osmotic or mechanical pressure may force the ink from the reservoir through the microneedles into the skin.

In another embodiment, the tattoo applicator 110 includes a needleless tattoo applicator configured to inject ink into the skin with high velocity ink streams.

Other example configurations of the tattoo applicator 110 are described below.

Modifications, additions, and/or omissions may be made to FIG. 1A without departing from the scope of the present disclosure. For example, the tattoo reader 106 may include multiple optical imagers 116 and/or multiple radiation sources 118. Alternately or additionally, the tattoo reader 106 may include a processor and/or may be configured to process data generated by the optical imager 116. Alternately or additionally, the tattoo reader 106, the health monitor application 112, and/or the animal records 114 may be included in or directly coupled to a single computing device as illustrated in FIG. 1B.

FIG. 1B is a diagram of another example system 100B to monitor the one or more animals 102 of FIG. 1A, arranged in accordance with at least some embodiments described herein. As illustrated in FIG. 1B, the tattoo reader 106, the health monitor application 112, and the animal records 114 may be included in or directly coupled to a computing device 120. The computing device 120 may include a handheld computing device such as a smartphone. Alternately or additionally, the computing device 120 may include a personal digital assistant, a laptop computer, a desktop computer, or other suitable computing device.

The tattoo reader 106, the health monitor application 112, and the animal records 114 illustrated in FIG. 1B may generally be configured and may operate in the same or similar manner as described with respect to FIG. 1A.

FIGS. 1A and 1B illustrate two operating environments in which embodiments described herein may be implemented. Alternately or additionally, a person such as a farmer, farmhand, rancher, ranch hand, veterinarian, or other person may measure, perceive, or otherwise observe the tattoos of the animals 102 with the naked eye for lifecycle monitoring. The person may record physiological status of the animals 102 manually in a book, notebook, journal, diary, and/or in a computing device. Alternately or additionally, the physiological status of the animals 102 as indicated by the tattoos of the animals and as observed by the person may inform how the person manages and cares for the animals 102. For example, the person may observe the tattoo, determine a physiological status of the animal 102 based on the tattoo, and then take some other action, such as treating a physiological event or condition indicated by the determined physiological status, separating the animal 102 from other animals 102 that do not have the same physiological event or condition, or some other action.

FIG. 2 illustrates an example animal 200 and placement of one or more tattoos 202 with intelligent ink on the animal 200, arranged in accordance with at least some embodiments described herein. The animal 200 of FIG. 2 is an example of the animals 102 of FIGS. 1A-1B.

The tattoos 202 include tattoos 202A, 202B, 202C, and 202D. While four tattoos are illustrated in FIG. 2, more generally one or more tattoos with intelligent ink may be applied to the animal 200 at the same or different locations than are illustrated.

In FIG. 2, the tattoo 202A may be applied to an inside of an ear of the animal 200, the tattoo 202B may be applied to a nose of the animal 200, the tattoo 202C may be applied to a foreleg of the animal 200, and/or the tattoo 202D may be applied to an udder of the animal 200. Tattoos may alternately or additionally be applied to other locations of the animal 200, such as the forehead, shoulder, hind leg, hindquarter, or other suitable location.

A location on the animal 200 to which a tattoo is applied may be cleaned, shaved, and/or otherwise prepared prior to application of the tattoo. Some locations of an animal may be easier to tattoo than other locations. For example, the animal 200 in FIG. 2 includes a cow, and locations such as the inside of ears, the nose, and the udder may naturally have less hair and may be easier to tattoo than other locations, such as the foreleg.

The tattoos 202 may be applied to the animal 200 at birth, at maturity, or at any other time. The tattoos 202 may be applied using manual, semi-automatic, or fully automatic tattoo applicators, some examples of which are described elsewhere herein.

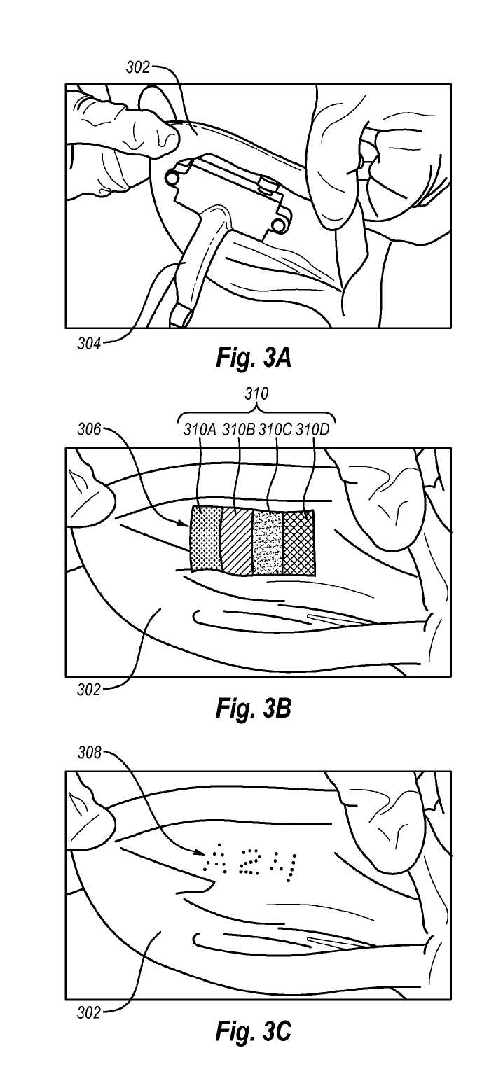

FIGS. 3A-3C illustrate an example animal ear 302, tattoo applicator 304, and tattoos 306 and 308 that may be applied to the animal ear 302, arranged in accordance with at least some embodiments described herein. The tattoos 306 and 308 are examples of tattoos that may be applied to an animal, such as the animals 102 and 200 of FIGS. 1A-2.

The tattoo applicator 304 of FIG. 3A is illustrated as a manual plier-type tattoo applicator. Other tattoo applicators may alternately or additionally be implemented. The tattoo applicator 304 may be configured to apply a tattoo to the animal ear 302, such as the tattoo 306 of FIG. 3B and/or the tattoo 308 of FIG. 3C.

The tattoo 306 of FIG. 3B includes four different inks 310A-310D (collectively “inks 310”). More generally, the tattoo 306 may include one or more different inks where at least one of the one or more different inks includes an intelligent ink. In some embodiments, each of the inks 310 may include an intelligent ink. For example, all four of the inks 310 may include a different intelligent ink.

Alternately, at least one of the inks 310 may include a non-intelligent ink used as a static reference or a baseline against which one or more of the other inks 310 may be compared. For example, the ink 310A and the ink 310C may each include a different intelligent ink while the ink 310B and the ink 310D may each include a different non-intelligent ink. The ink 310A may be compared against the ink 310B as a static reference or baseline, while the ink 310C may be compared against the ink 310D as a static reference or baseline.

In some embodiments, two or more non-intelligent inks may be used as a static reference or baseline for a single intelligent ink. As an example, the ink 310B may include an intelligent ink configured to respond to a target of physiology by changing between two colors dependent on changes to the target. In this example, the ink 310A may include a non-intelligent ink that is one of the two colors while the ink 310C may include a non-intelligent ink that is the other of the two colors. Accordingly, measurements of the ink 310B may be compared against measurements of the inks 310A and 310C to determine whether, at any given time, the ink 310B is closer in color to the ink 310A or the ink 310C, which may be indicative of a physiological status of the animal.

The intelligent inks described herein may be configured to undergo reversible or irreversible changes dependent on the particular target of physiology to which the intelligent inks are configured to respond, as manifested by reversible or irreversible changes in the measured optical property of the intelligent inks. For example, an optical property, such as color saturation, of an intelligent ink may be configured to reversibly vary dependent upon variations of the corresponding target of physiology, such as blood glucose level. The color saturation or other optical property may vary up and down as the blood glucose level varies up and down and may thus be measured repeatedly over time to monitor the blood glucose level. Thus, a reversible intelligent ink may undergo a reversible change in response to the corresponding target of physiology satisfying a particular criteria, but the reversible change may not be measurable unless the particular criteria is satisfied at the time of measurement.

Alternately, an optical property, such as color, of an intelligent ink may be configured to irreversibly change from an initial value to another value in response to the corresponding target of physiology, such as temperature, reaching or exceeding a particular threshold. Accordingly, the color or other optical property may irreversibly change from an initial color or value to another color or value in response to the temperature or other target reaching or exceeding a particular threshold, such as 41.0° C., which may indicate that the animal has or has had a fever or other physiological event or condition. Intelligent inks that undergo irreversible changes may be used to identify the occurrence of a transitory physiological event or condition or other physiological event or conditions at any time during or after the occurrence of the physiological event or condition, whether or not such a physiological event or condition exists at the time the intelligent ink is read. Thus, an irreversible intelligent ink may undergo an irreversible change in response to the corresponding target of physiology satisfying a particular criteria at least once, and the irreversible change may be measurable whether or not the particular criteria is satisfied at the time of measurement.

The tattoo 306 of FIG. 3B may generally be used for lifecycle monitoring. Alternately or additionally, tattoos with intelligent ink may be used to uniquely identify animals. For example, the tattoo 308 of FIG. 3C includes a 3-digit alphanumeric code to uniquely identify the animal to which the tattoo 308 is applied. Different 3-digit alphanumeric codes may be used on different animals to uniquely identify those animals. More generally, tattoos used to uniquely identify animals may include any combination of numbers, letters, and/or symbols to uniquely identify the animals. The alphanumeric code and/or other elements of the tattoo that uniquely identify each animal may be referred to as the animal's ID.

In FIG. 3C, each of the three digits of the tattoo 308 may include an intelligent ink and/or a non-intelligent ink. For instance, at least one of the three digits of the tattoo 308 may include an intelligent ink. Each of the three digits may include the same intelligent ink, or at least one of the three digits may include a different intelligent ink than the other digits. Alternately or additionally, at least one of the three digits of the tattoo 308 may include a non-intelligent ink to be used as a static reference or baseline.

Various examples of intelligent inks will now be described.

Intelligent inks configured to respond to antigens in an animal may include UV-irradiated colored polydiacetylene (PDA) vesicles coupled with antibodies or other antigen receptors. The antigens to which the intelligent ink is configured to respond may be specific to particular hormones or may otherwise be indicative of hormone levels of the particular hormones, such as cortisol or progesterone levels, in the animal. Accordingly, the antigens may include hormone-specific antigens and the antibodies conjugated to the PDA vesicles may include hormone-specific antibodies. For example, the antibodies may include cortisol-specific antibodies to detect cortisol-specific antigens.

The PDA vesicles coupled with hormone-specific antibodies may be referred to as sensors. A distinct color change may be produced on the PDA vesicles when the hormone-specific antibodies are exposed to a corresponding hormone-specific antigen in solution. Biologically-produced hormone-specific antibodies can be conjugated to the PDA vesicles to produce the intelligent ink. A color change of the intelligent ink from blue to red in the presence of the hormone-specific antigens may be clear and sensitive. The color change may be due to immunoreaction at a surface of the PDA vesicle, which may result in a conformational change in the PDA vesicle. The color change in this example may be visible to the naked eye from concentrations as low as 100 ng/mL, 10 ng/mL, or 1 ng/mL. Concentrations as low as 100 ng/mL, 10 ng/mL, or 1 ng/mL may be sufficiently sensitive to be used as a threshold sensor for blood concentration of cortisol or progesterone, for example. However virtually any antigen could be detected using such an intelligent ink by conjugating an appropriate antibody, antibody mimetic or receptor molecule to the PDA vesicle. Tuning the sensitivity of these systems may be accomplished through the addition of various amounts of lipids into membranes of the PDA vesicle: the type and concentrations of lipids can alter the sensitivity.

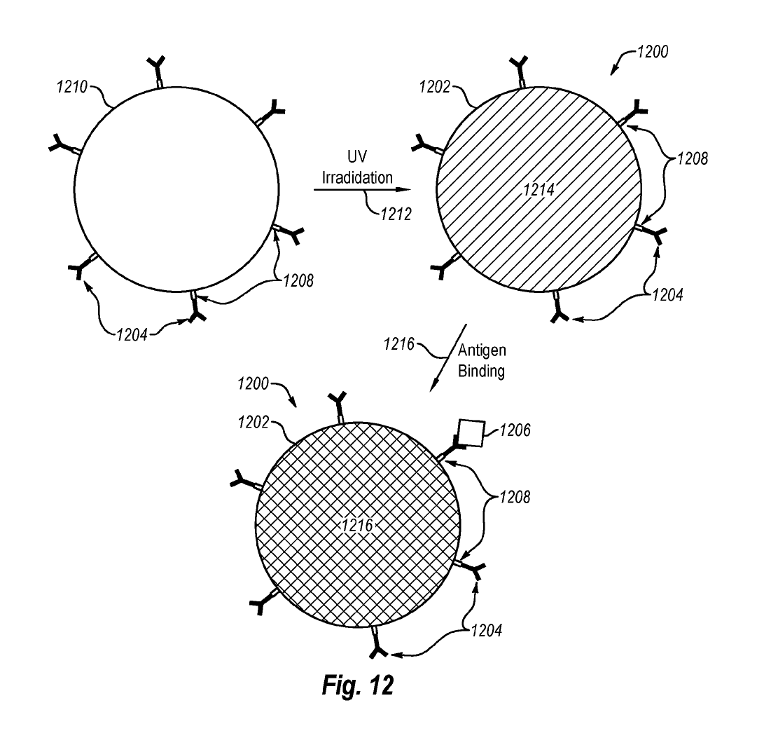

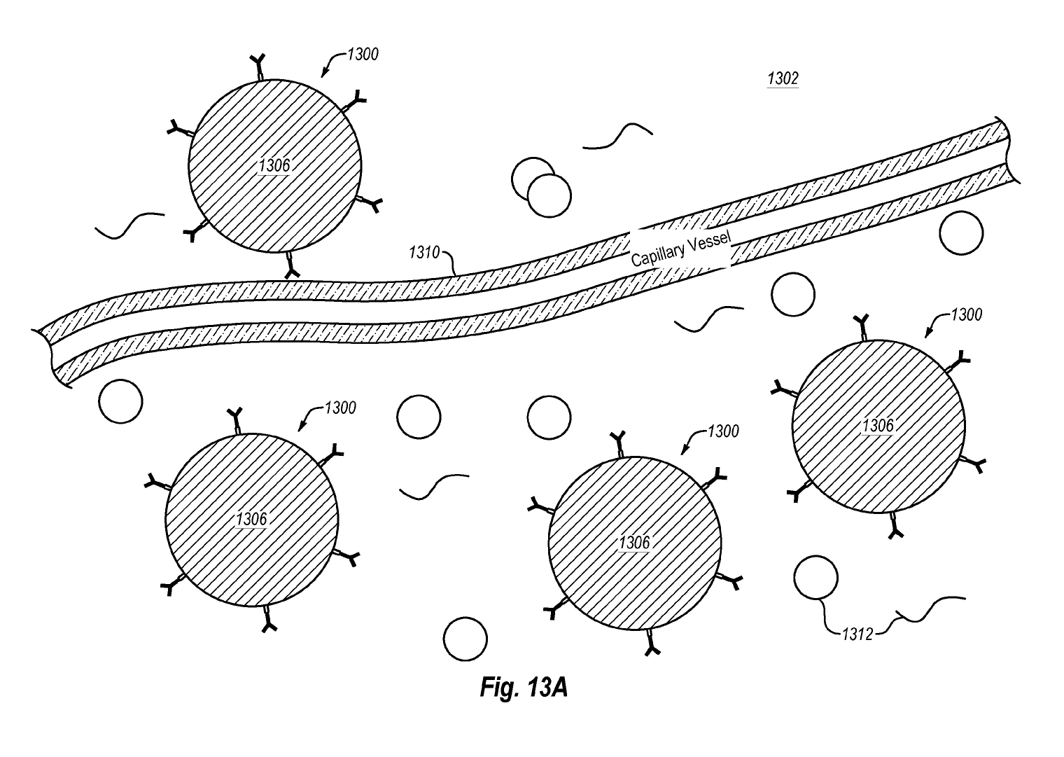

FIG. 12 illustrates an example sensor 1200 that includes a PDA vesicle 1202 conjugated with multiple antigen receptors 1204, arranged in accordance with at least some embodiments described herein. The sensor 1200 may be included in an intelligent ink that includes multiple sensors 1200.

Only some of the antigen receptors 1204 are labelled in FIG. 12 for simplicity. The antigen receptors 1204 may include antibodies, antibody mimetics, or receptor molecules specific to a particular antigen 1206. Each of the antigen receptors 1204 may be bound to the PDA vesicle 1202 with a peptide bond 1208, only some of which are labelled in FIG. 12 for simplicity.

The sensor 1200 may be formed from a diacetylene lipid vesicle 1210 conjugated with the antigen receptors 1204. Irradiation of the diacetylene lipid vesicle 1208 with UV light, as indicated at 1212, may change the diacetylene lipid vesicle 1210 to the PDA vesicle 1202.

The sensor 1200 and/or the PDA vesicle 1202 may have a variable optical property that is indicative of a presence or absence of the antigen 1206. The variable optical property may include color and/or fluorescence. For example, in an absence of the antigen 1212, the sensor 1200 may be blue in color without fluorescence, as indicated by hatch pattern 1214. When the antigen 1212 is present, e.g., within a vicinity of the sensor 1200, it may bind to one of the antigen receptors 1204, as indicated at 1216. The PDA vesicle 1202 may undergo a conformational change responsive to the antigens 1212 binding to the antigen receptors 1204. The conformational change of the PDA vesicle 1202 may cause the color of the sensor 1200 to change to red, as indicated by hatch pattern 1216. The sensor 1200 may also exhibit fluorescence when its color is red. Thus, the color and/or fluorescence of the sensor 1200 may be indicative of the presence or absence of the antigen 1206.

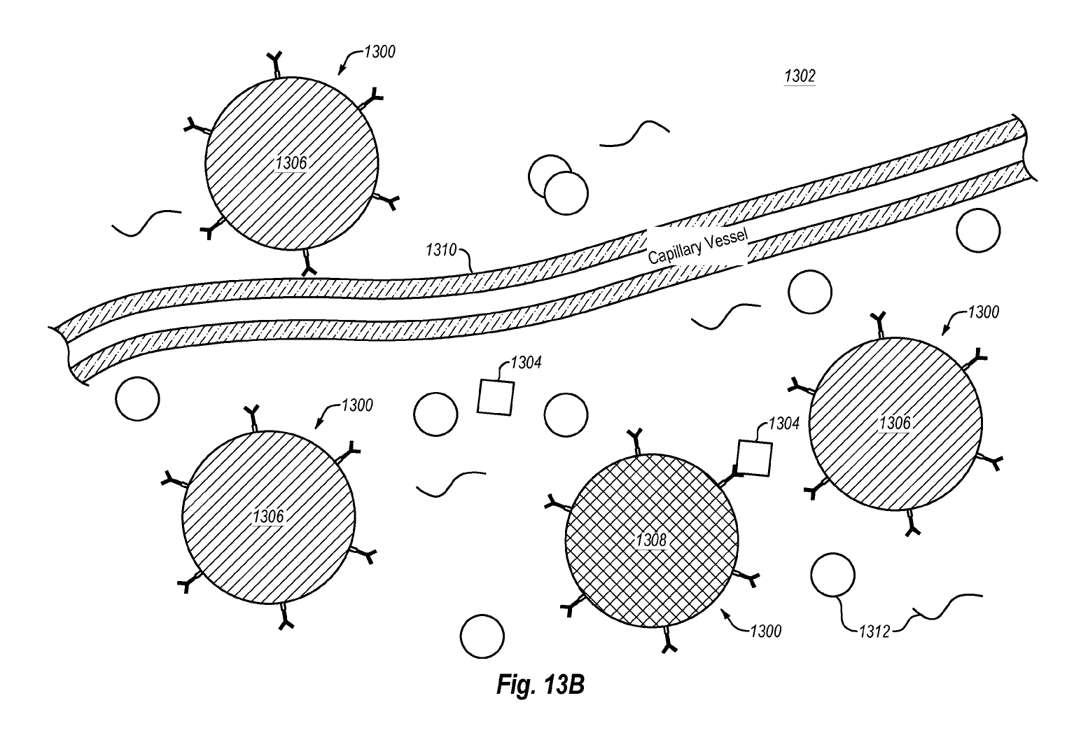

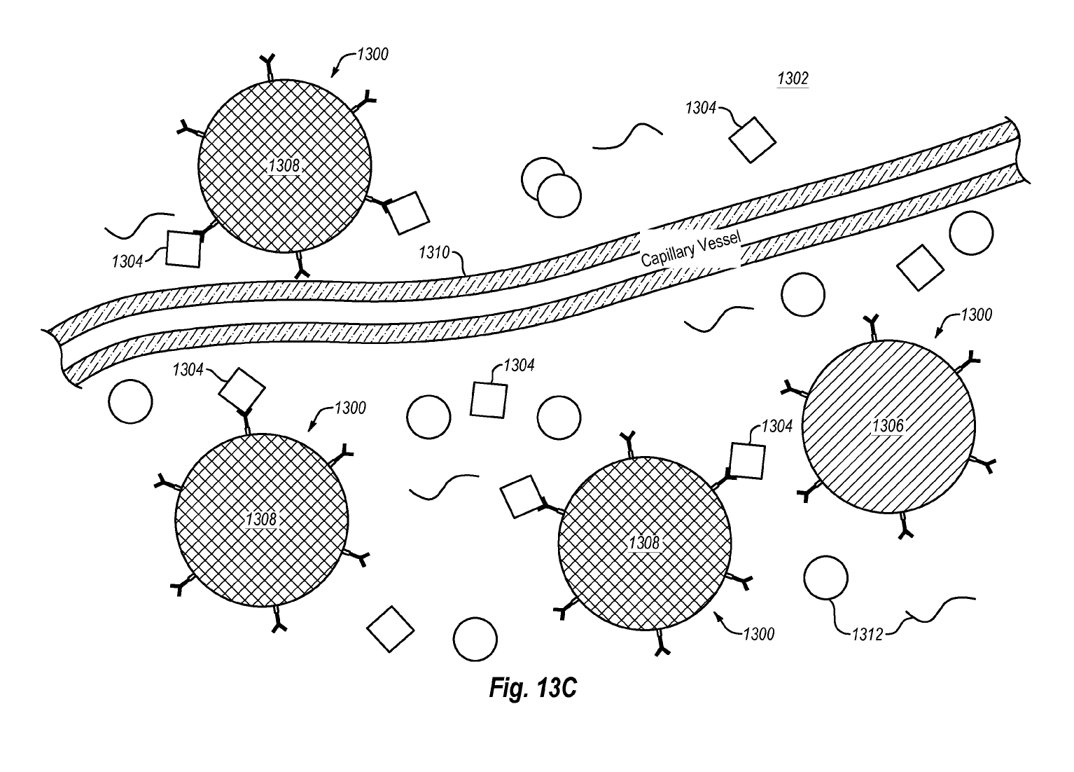

FIGS. 13A-13C depict operation of sensors 1300 injected into dermis 1302 of an animal, arranged in accordance with at least some embodiments described herein. The sensors 1300 may be similar or identical to the sensor 1200 of FIG. 12. Each of the sensors 1300 may have a variable optical property that is indicative of a presence or absence of antigens 1304 (FIGS. 13B and 13C) particular to antigen receptors included in each of the sensors 1300. In an absence of the antigens 1304, the sensors 1300 may have one color and/or fluorescence state, as indicated by a hatch pattern 1306. When the antigens 1304 are present and bind to the sensors 1300, the sensors 1300 may have a different color and/or fluorescence state, as indicated by a different hatch pattern 1308 (FIGS. 13B and 13C). Although four sensors 1300 are illustrated in FIG. 13A, the dermis 1302 may more generally have injected therein one or more sensors 1300.

One or more capillary vessels 1310 that passes through or near the dermis 1302 may supply blood to the dermis 1302. The blood may include one or more solutes 1312 that are not relevant to the sensors 1300, e.g., which are not detected by the sensors 1300, and which may be referred to as non-relevant solutes 1306.

As illustrated in FIG. 13A, and in an absence of the antigens 1304 (FIGS. 13B and 13C), the sensors 1300 may have the color and/or fluorescence state associated with the hatch pattern 1306.

As illustrated in FIG. 13B, when the antigens 1304 are present in a relatively low concentration in the blood supplied by the capillary vessel 1310 to the dermis 1302, the antigens 1304 may bind to relatively few of the sensors 1300. The sensors 1300 to which the antigens 1304 bind may have the different color and/or fluorescence state associated with the different hatch pattern 1308. When the concentration of antigens 1304 is relatively low such that relatively few of the sensors 1300 have the different color and/or fluorescence state associated with the different hatch pattern 1308, it may be difficult to perceive any change to an aggregate color of a tattoo that includes the sensors 1300. Alternatively or additionally, where some of the sensors 1300 have one color and/or fluorescence state, such as blue color, and others of the sensors 1300 have the different color and/or fluorescence state, such as red color, an aggregate color of the tattoo that includes the sensors 1300 may include a combination of the two colors, such as purple color.

As illustrated in FIG. 13C, and in comparison to FIG. 13B, when the antigens 1304 are present in a relatively higher concentration in the blood supplied by the capillary vessel 1310 to the dermis 1302, the antigens 1304 may bind to relatively more of the sensors 1300. Only some of the antigens 1304 are labeled in FIG. 13C for simplicity. The sensors 1300 to which the antigens 1304 bind may have the different color and/or fluorescence state associated with the different hatch pattern 1308.

A tattoo ink that incorporates an intelligent ink with PDA vesicles such as are included in the sensors 1200 and 1300 of FIGS. 12-13C may be delivered in a carrier, such as phosphate buffered saline (PBS). In some embodiments, the carrier may provide flow and/or may match conditions (e.g., pH and osmolarity) of an injection site. Optionally, the tattoo ink may also include other ink elements, such as factors to facilitate and/or improve measurement of a tattoo that includes the intelligent ink. For example, the tattoo ink may also include vascular endothelial growth factor (VEGF), which may be associated with the PDA vesicles or which may be free in the carrier, to recruit blood vessels to the vicinity of the sensors after the tattoo is injected. As another example, the tattoo ink may also include one or more agents to promote healing and reduce scar formation around the tattoo ink, such as epidermal growth factor (EGF), or one or more opacity-enhancing agents or other agents to improve visualization, or one or more support elements such as hydrogel scaffolds.

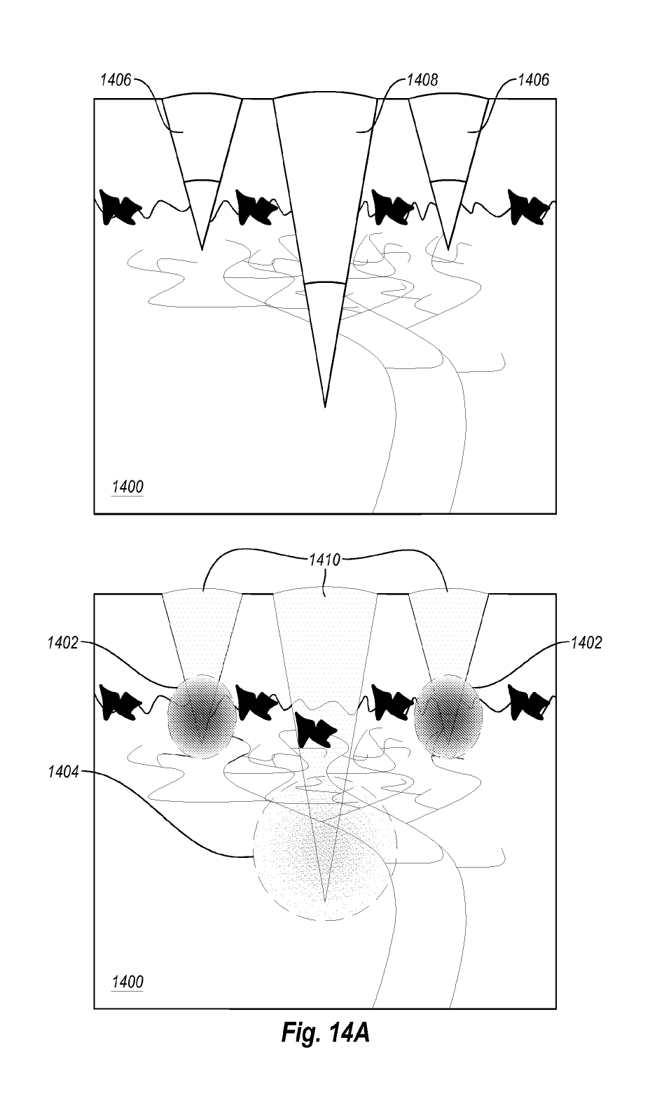

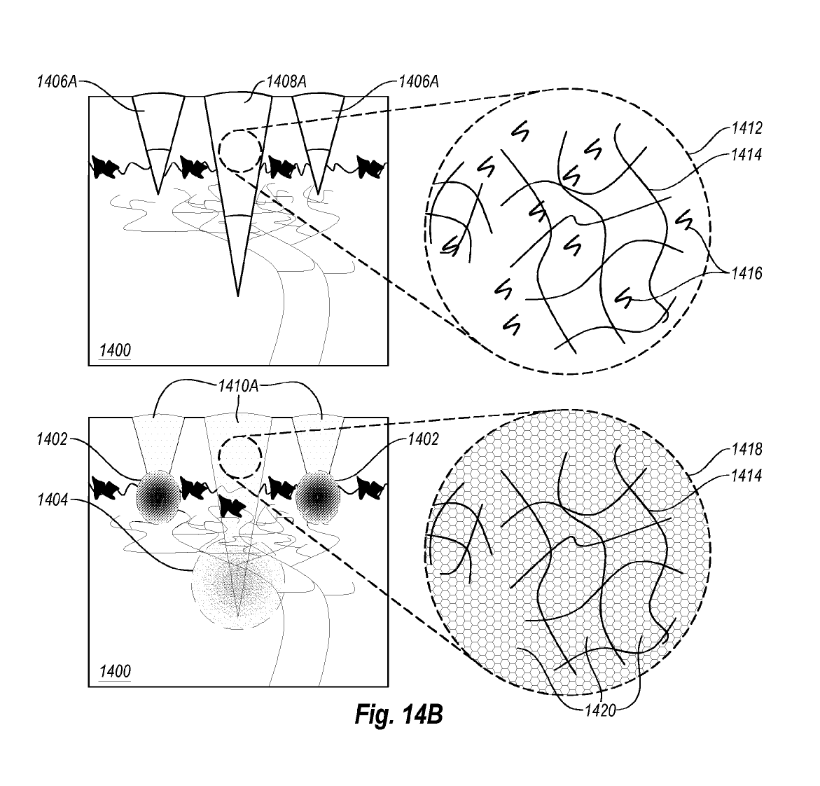

FIGS. 14A and 14B depict injection of a tattoo ink into skin 1400 of an animal, arranged in accordance with at least some embodiments described herein. In FIG. 14A, the tattoo ink may include intelligent ink. In FIG. 14B, the tattoo ink may include intelligent ink and one or more other ink elements.

In more detail, in FIG. 14A, the tattoo ink includes intelligent inks 1402 and 1404 delivered by needles 1406 and 1408 that inject the intelligent inks 1402 and 1404 into the skin 1400. The needles 1406 and 1408 may be removed immediately after injecting the intelligent inks 1402 and 1404 into the skin 1400, or the needles 1406 and 1408 may be dissolved in and absorbed by the skin 1400 to release the intelligent inks 1402 and 1404 into the skin 1400. As the skin 1400 heals after injection of the intelligent inks 1402 and 1404 by the needles 1406 and 1408, the skin 1400 may form relatively dense epidermal scar tissue 1410 where it experienced trauma from penetration of the needles 1406 and 1408. The relatively dense scar tissue 1410 may be relatively opaque and may decrease visibility of the intelligent inks 1402 and 1404 and their associated optical properties.

In FIG. 14B, the tattoo ink includes the intelligent inks 1402 and 1404 and one or more other ink elements delivered by needles 1406A and 1408A. As illustrated in detail view 1412, the one or more other ink elements may include a hydrogel scaffold 1414 and EGFs 1416, only some of which are labeled in FIG. 14B for simplicity. The hydrogel scaffold may include a crosslinked hydrogel collagen scaffold in some embodiments.

The hydrogel scaffold 1414, the EGFs 1416, and/or other ink elements may be provided together with the intelligent inks 1402 and 1404 at a same depth in the needles 1406A and 1408A to deliver the intelligent inks 1402 and 1404 to a common depth in the skin 1400. For example, VEGF may be provided at the same depth in the needle 1408A as the intelligent ink 1404 to induce vasculogenesis and/or andiogenesis in a vicinity of the intelligent ink 1404, as denoted at 1418 in FIG. 14B. As a result, blood flow in the vicinity of the intelligent ink 1404 may be improved, which may improve sensitivity of the intelligent ink to a target of physiology that includes a blood marker.

Alternatively or additionally, the hydrogel scaffold 1414, the EGFs, and/or other ink elements may be provided in the needles 1406A and 1408A at a different depth or depth than the intelligent ink 1402 or 1404. For example, the intelligent ink 1404 may be provided in a tip of the 1408A that penetrates to dermis of the skin 1400, while the hydrogel scaffold 1414 and the EGF 1416 may be provided at a depth of the needle 1408A that penetrates to epidermis of the skin 1400 to deliver the hydrogel scaffold 1414 and the EGF 1416 to the epidermis of the skin 1400. As illustrated in detail view 1418, epidermal growth cells may infiltrate the hydrogel scaffold 1414, promoted by the EGF 1416. In FIG. 14B, the hydrogel scaffold 1414 and the EGF 1416 may cause the skin 1400 to heal with relatively less epidermal scar tissue 1410A compared to FIG. 14A.

Returning to the discussion of sensors that include PDA vesicles conjugated with antibodies, after an intelligent ink that includes such sensors is injected in the skin to form the tattoo, the intelligent ink may alter color from blue to red in response to binding of the hormone-specific antigen to the hormone-specific antibodies conjugated to the PDA vesicles. The color change may be readable by the naked eye, however, it is also possible to be machine read, e.g., by the tattoo reader 106 of FIGS. 1A-1B or other suitable tattoo reader. For example, the optical imager 116 of the tattoo reader 106 may include a digital camera to take a picture that a computer, e.g., the server 104 and/or the computing device 120 of FIGS. 1A-1B, can process to determine the color(s) in the tattoo. Alternatively, the optical imager 116 of the tattoo reader 106 may include a spectrophotometer that can measure absorbance of light of the tattoo to determine what the color of the tattoo is at a given point in time. The determined color information can then be correlated with the animal's ID.

PDA alternates between a non-fluorescent state (when it is blue) to a fluorescent state (when it is red). As such, the intelligent ink in this example can also be read by measuring fluorescence of the intelligent ink. To measure the fluorescence, an appropriate excitation illumination may be provided, e.g., by the radiation source 118 of FIG. 1A. When the PDA of the intelligent ink is red, exposure of the PDA to the excitation illumination may cause the PDA to fluoresce. The fluorescence may be measured using a fluorescence spectrophotometer or other suitable device such as may be included in the optical imager 116 of FIG. 1A, for example.

The concentration of the intelligent ink to be visible to the naked eye and/or to be measured by the tattoo reader 106 of FIGS. 1A-1B may depend on one or more factors, such as depth of the tattoo, type of animal or the injection site on its hide, area of the tattoo, and whether the color or fluorescence is measured. However, if the intelligent ink is to be read by the naked eye, a 1 square centimeter (cm) area being tattooed with the intelligent ink may include between 1 milligram (mg) and 100 mg of PDA vesicles, or between 40 mg and 100 mg, or between 80 mg and 100 mg. A size of the area being tattooed and a concentration of the intelligent ink in the area being tattooed may be adjusted, as needed.

In some embodiments, the intelligent ink may be stabilized in the hide of the animal for an extended time, e.g., for several months or even a year or more. In this case, the PDA vesicles of the intelligent ink may be encapsulated in a semipermeable encapsulant to reduce their deterioration and resorption compared to non-encapsulated embodiments. Alginate encapsulation for xenotransplants affords a stable environment for material that would otherwise be attacked by a host's immune system and quickly removed. The alginate may be biocompatible and can be tuned to an appropriate molecular weight cutoff to ensure passage of molecules of interest (e.g., the hormone-specific antigens) into, and out of, the vicinity of the sensor. For example, with a PDA vesicle which is conjugated to a hormone-specific antibody that specifically binds cortisol, a molecular weight cutoff of about 1 kilodalton (kDa) may be sufficient to allow relatively free diffusion of cortisol while preventing ingress into a vicinity of the sensor of enzymes that have a molecular weight above the molecular weight cutoff. Such enzymes may include proteases or other enzymes that may degrade the hormone-specific antibody.

The sensors may be encapsulated in a time-release encapsulant which may be semipermeable or impermeable. The time-release encapsulant may degrade over a duration of time to eventually expose the sensor within. The duration of time may include a duration of time on the order of seconds, minutes, hours, days, weeks, months, or some other duration. For example, the duration of time may include 24 hours, 30 days, 45 days, 90 days, or some other duration of time. In some embodiments, different sets of sensors in the intelligent ink may be encapsulated in time-release encapsulants of different durations of time to stagger exposure of the different sets of sensors over time. If the time-release encapsulant is semipermeable, the sensors may operate as described above while encapsulated. After degradation of the semipermeable time-release encapsulant, the sensors may be exposed to deterioration and resorption within the animal, which may eventually cause the tattoo with the intelligent ink, or at least some portion of the intelligent ink of the tattoo, to fade or disappear if the exposed intelligent ink is not otherwise stabilized within the hide of the animal. If the time-release encapsulant is impermeable, the sensors may generally be inactive while encapsulated within the impermeable time-release encapsulant. After degradation of the impermeable time-release encapsulant, the sensors may be activated to sense antigens or other compounds. The encapsulation of the sensors in time-release encapsulants may be used to cause automatic fading or disappearance of the tattoo over time, time-delayed activation of some or all of the sensors, or some other outcome.

The intelligent ink may be embedded into capillary beds of the dermis to ensure significant perfusion in some embodiments. As mentioned above, the intelligent ink may also have factors to increase perfusion of the sensors by encouraging growth of capillaries in the vicinity of the tattoo. The dermis may provide contact with capillary beds, while still being close enough to a surface of the animal's hide to ensure visibility of the sensors. The epidermis may provide a protective layer over the tattoo that aids in stabilizing it over time. Alternatively, the tattoo may be embedded more towards the epidermis, within the epidermis, or even beneath the dermis, such as within the hypodermis or subcutis. In some cases, the tattoo may be embedded into multiple layers within the animal's skin structure.

Agents may be added to the intelligent ink that aid in the visualisation of the sensors. Alternately or additionally, the agents may be delivered separately to the same injection site as the intelligent ink. The agents may include depigmentation agents (e.g., monobenzone) that effectively remove melanin or other pigments present at the tattoo injection site, depilatory agents that remove hair (e.g., thioglycolic acid), or other agents. Such agents may be injected into the same or different skin layers as the sensors of the intelligent ink. For example, the intelligent ink may be injected into the dermis to contact the capillary beds, while depigmentation agents may be injected into the epidermis to act on melanocytes present there and/or depilatory agents may be applied to a surface of the skin or may be injected to a layer within the dermis or hypodermis where hair follicles reside.

Strategies for generating this and other intelligent inks may utilize generally recognized as safe (GRAS) chemistry such that the intelligent inks may be used in production animals (which may enter the food chain) and/or in humans. Furthermore, this and other intelligent inks may have a shelf life of several months or more, or could be stabilized with preservatives such as antioxidants to ensure a shelf life of several months or more. The tattoo itself (and the intelligent ink thereof) may be stable for years after injection into the skin. Additional agents included in the intelligent ink or otherwise applied at or in the same injection site as the tattoo may extend the life of the tattoo even further, such as UV blocking agents (e.g., titanate) to reduce degradation due to sun exposure.

Intelligent inks other than those described above may respond to antigens in the animal. For example, the intelligent ink may include cyclodextrin functionalized with a dye chromophore and modified to preferentially accept a target hormone. The dye chromophore may include methyl red, the color of which may be suppressed by cyclodextrin unless the target hormone is present within the cyclodextrin as a guest where the dye chromophore may appear orange under subcutaneous pH. This intelligent ink may not amplify the hormone signal, and may not be highly sensitive. As such, this intelligent ink may be used to detect incremental cumulative increases in hormone levels, rather than nanomolar thresholds.

Another intelligent ink may include antibody or aptamer functionalized gold nanoparticles in polymer encapsulated solution. These particles may create a strong structural color when individually suspended. This structural color may be altered, generally from red to blue, when a corresponding target molecule binds to the surface antibody and causes agglomeration of the nanoparticles. This intelligent ink may have a magnifying effect, as one binding target molecule can agglomerate several nanoparticles. Gold nanoparticle surface aptamers for cortisol and similar molecules exist and can be used in the intelligent ink to detect cortisol or other similar molecules. This intelligent ink can detect concentrations as low as 40 ng/mL, 30 ng/mL, or 20 ng/mL for cortisol, for example. High sensitivity combined with low level amplification lead to good performance of this intelligent ink as a threshold test.

Another intelligent ink may include an encapsulation of a hormone antibody conjugated to alkaline phosphatase. This conjugation may inhibit an enzyme function. The encapsulation may include a standard phosphatase stain e.g. Fast red violet—Napthol AS-BI phosphate, which may remain inactivated until hormone-antibody binding occurs. Phosphatase activity may be restored in response to the hormone-antibody binding and the stain may be activated.

Another intelligent ink may include a bacterial cell line that may be a simple and controllable hormone responsive line that may have both a tunable amplification response and a controllable expression. For example, E. coli strains can be engineered to respond to many target molecules, and to amplify the signal in a tunable manner. Natural bacterial response to steroids exists, and can be genetically transferred to E. Coli via plasmid. This response may occur down to at least 0.5 mM, or down to a nM range or a pM range. A simple form of color change may include expression of bleaching enzymes into an enzyme sensitive dye filled encapsulant such as Remazol Black-B.

Another intelligent ink may include an immortalized chromatophore or melanophore cell line from fish, such as Betta splendins, to sense and provide a color or shade response to specific hormones. The target specificity may be produced by modification of existing responses within the cell as melanophores respond to steroid concentrations. These cells may be encapsulated where they are still capable of receiving blood solutes.

Another intelligent ink may include encapsulated tissue engineered from animal chromatophore organ to be responsive to the target hormone, similar to the operation of cellular level modified melanophores. Squid, octopus and some bony fish possess organ tissue that can be used for this purpose. The tissue may be well suited to propagation and modification.

Another intelligent ink may include particles possessing a 1-, 2-, or 3-dimensional photonic crystal structure, such as inverse opal, constructed from molecularly imprinted polymer (MIP) with target hormone imprints. The finely tuned photonic structure may produce a specific structural color, dependent on spacing of porous elements. The MIP may take up the target molecule into its imprints and undergo a process of expansion, which may cause a change in spacing and thus a visible change in the structural color of the component. MIP uptake of progesterone and cortisol have been developed. The components can detect analyte concentrations below 50 ng/L.

Another intelligent ink may include particles that include an encapsulated orientation controlled liquid crystal film over hormone antibodies immobilized on a permeable polymer element that allows hormone access to the hormone antibodies. The liquid crystals may be vertically aligned when hormone antibodies are unbound to the target antigen and thus appear dark. When antigen binding occurs, the liquid crystals may rotate and become highly reflective of polarized light.

Intelligent inks configured to respond to a target of physiology of the animal, such as temperature, may include thermochromic leuco dyes that reversibly or irreversibly bleach or color over a temperature range. An example leuco dye suitable for use as an intelligent ink may include crystal violet lactone with temperature controllable protonation. The leuco dye may be encapsulated in a microcapsule with an organic acid salt and a solvent selected for a specific melting point (m.p.) to achieve about a 39° C. transition point (or other suitable transition point) for the color/bleaching change of the leuco dye. As a particular example, the leuco dye may be encapsulated with, e.g., fatty alcohol mixtures such as myristyl alcohol (m.p. 38° C.) with a low percentage of cetyl alcohol (m.p. 49° C.). The 39° C. transition point in this example may be suitable for use in cattle with a standard body temperature in adult cattle of about 38-38.5° C. In this example, in response to melting of the solvent, the solutes may become mobile and the pH may drop, which may produce a structural change in the leuco dye that causes a color change within the microcapsule. The microcapsule may be referred to as a sensor.

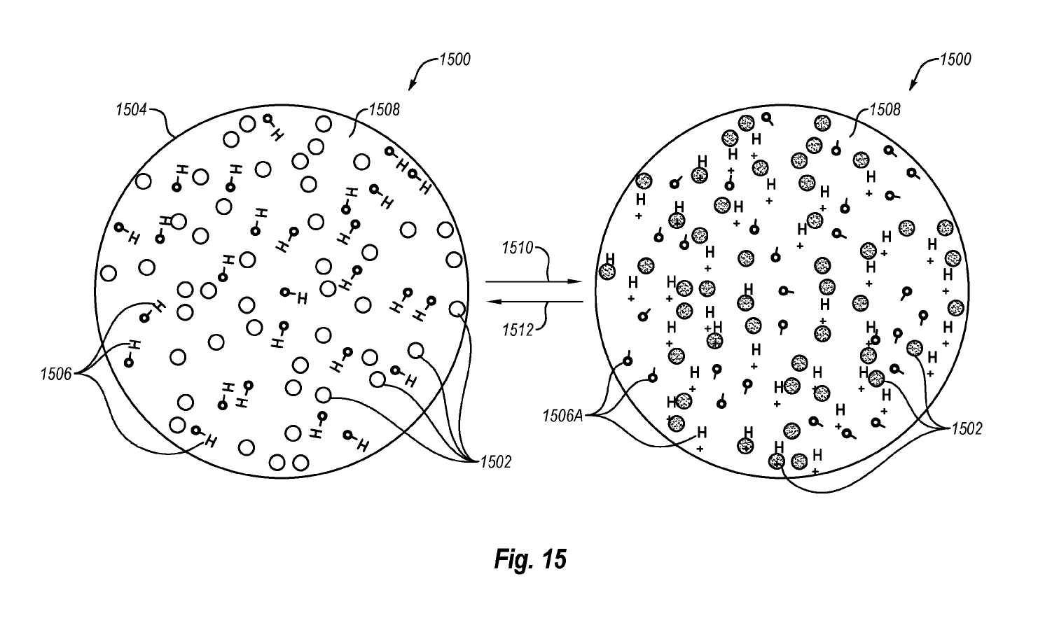

FIG. 15 illustrates an example sensor 1500 that includes leuco dye 1502, arranged in accordance with at least some embodiments described herein. The sensor 1500 includes a microcapsule 1504 that encapsulates the leuco dye 1502, an organic acid salt 1506, and a solvent 1508 with a melting point. At temperatures below the melting point of the solvent 1508, the solvent 1508 may be solid, the organic acid salt 1506 may be immobile, and the leuco dye 1502 may be colorless, as indicated by an absence of a hatch pattern in the leuco dye 1502 of the microcapsule 1500 on the left side of FIG. 15. As a result, the microcapsule 1500 may appear colorless at temperatures below the melting point of the solvent 1508.

If the temperature increases to a temperature above the melting point, as denoted at 1510, the solvent 1508 may melt and the solutes, e.g., the organic acid salt 1506, may become mobile and may transition to a dissociated acid 1506A. The mobile dissociated acid 1506A may cause the pH to drop, which may produce a structural change in the leuco dye 1502, e.g., protonation of the leuco dye 1502, that causes a color change in the leuco dye 1502 from colorless to a violet color, as indicated by a hatch pattern included in the leuco dye 1502 in the microcapsule 1500 on the right side of FIG. 15. Lowering the temperature below the melting point of the solvent 1508, as denoted at 1512, may reverse the process by solidifying the solvent 1508 to cause the dissociated acid 1506A to transition to the organic acid salt 1506 and raise the pH and thereby unprotonate the leuco dye 1502.

Encapsulation in FIG. 15 and other embodiments may be performed within polyester or polystyrene spheres on the order of 3-5 micrometers (μm), or within other suitable inert polymer microcapsules.

The chemistry of the intelligent ink described above (e.g., encapsulated leuco dye with organic acid salt and solvent) may provide a reversible change. Some leuco dyes are available that may undergo irreversible color changes and provide permanent recording of temperature spikes. A range of these leuco dyes are available off the shelf.

Intelligent inks that include leuco dyes that undergo reversible or irreversible color changes may be visible to the naked eye and/or may be measurable by a tattoo reader, such as the tattoo reader 106 of FIGS. 1A-1B. The change may include a change from a colorless and/or natural light skin tone to a dark intense violet color.

Concentrations of 1-250 mg, or 100-250 mg, or 200-250 mg of microcapsules per square cm of skin may be injected to produce a highly visible marker. The microcapsules may be delivered suspended in a skin absorbed carrier such as isopropyl myristate. A size of the area being tattooed and a concentration of the intelligent ink in the area being tattooed may be adjusted, as needed.

In this example, the active components (e.g., the leuco dye) in the microcapsules are stable and long-lived, and are encapsulated within an inert polymer such as a cellulose derivative, which may prevent the access of external agents. Accordingly, this intelligent ink may be capable of sensing relevant temperature changes within the skin of an animal for 2-3 years or for some other period of time.

The intelligent ink can be tailored to respond to relevant temperatures at a desired location by altering the composition balance of the encapsulated solvents. At a suitable depth in the skin and placement on the body of the animal the ink may have access to body heat for which its temperature responsive range can be calibrated. Depending on the location of the tattoo on the body, a core body temperature may be inferred from a previously established relationship between skin temperature and core temperature.

In some embodiments, the intelligent ink may be injected to a relatively shallow depth in the skin to detect skin temperature, which may be influenced by ambient temperature. In other embodiments, the intelligent ink may be injected to a relatively deeper depth, e.g., into a fat layer in the subcutis or below to detect core temperature and/or to reduce an influence of the ambient temperature on the intelligent ink.

The leuco dyes contained within the microcapsules may undergo reversible changes without being damaged by cycling from colored to colorless below about 60° C.

This intelligent ink may be placed anywhere within the dermis, which may include a depth of between 0.1 mm to 4 mm deep depending on the breed and species of the animal. The depth may vary if used in different animals or different sites on an animal. The significant darkening color change when the temperature of the injection site exceeds the transition point of the intelligent ink may make this intelligent ink strongly visible through the skin.

No further amplification may be needed to perceive (e.g., by the naked eye or using a tattoo reader) the change in the intelligent ink as the temperature change may affect all microcapsules individually.

The capsules may be inert, may contain no toxic components, and may be delivered only to the skin. These types of microcapsules have been employed in clothing and indicators on food and beverage containers. They may not present a health risk to animals or people, and any consumption or intake may be extremely low due to the placement and large size of the microcapsules.

The shelf life of this intelligent ink may be greater than 5 years under correct storage conditions, e.g., standard room temperature of about 25° C. and no direct sunlight.

Intelligent inks other than those described above may respond to temperature or other target of physiology of an animal. For example, the intelligent ink may include a hygroscopic anhydrous salt that gains or changes color upon hydration. The hygroscopic anhydrous salt may be encapsulated within a microcapsule of a polymer with side chains that crystallize at a different temperature, referred to as a transition point, to that of backbone chains. The transition point may be finely tunable. When the side chains are crystallized (e.g., at temperatures below the transition point) the polymer's water permeability may be extremely low. At temperatures above the transition point, the side chains may relax and allow passage of water vapor. The hygroscopic anhydrous salt, which may be colorless when not exposed to moisture, may be exposed to moisture when the side chains relax and allow passage of water vapor, thereby becoming colored. The hygroscopic anhydrous salts may include cobalt chloride, copper sulphate, or other hygroscopic anhydrous salt. The color change in this intelligent ink may be irreversible.

Another intelligent ink may include encapsulated thermochromic cholesteric or chiral nematic liquid crystal oils that are delivered into the cutis. The liquid crystal oils may be encapsulated by a number of techniques including coacervation with a compatible polymer. Cholesteric liquid crystals may be precisely tunable to specific transition temperatures, and transition ranges, and may be tuned to transition visibly and accurately from red to blue between 37° C. to 40° C. or some other temperature range. This intelligent ink may be used for continual temperature monitoring of animals, as a single piece of material may be capable of producing a range of colors for a temperature range, allowing identification of low, moderate and high temperatures and intermediates.

Another intelligent ink may include PDA polymers that undergo a color change from blue to red under rising temperatures between 23° C. to 130° C. A specific region of this range can be selected by altering an initial monomer for polymerization. This intelligent ink may be used to produce reversible or irreversible changes.

Another intelligent ink may include Astaxanthin bound to another protein. Astaxanthin is a brightly orange/red colored carotenoid when in a free state (e.g., when it is not bound to another protein). The color of Astaxanthin may be suppressed when not in the free state (e.g., when it is bound to another protein). The color may return after the bound protein has been denatured. Astaxanthin has a high denaturation temperature well above that achievable within the body of an animal, and thus cannot be thermally denatured within the body of the animal. However Astaxanthin can be bound to proteins that begin denaturing at ˜40° C., which is a property of many intracellular proteins, thus acting as a biologically relevant thermochromic indicator. The color change in this intelligent ink may be irreversible.

Another intelligent ink may include a compound of copper (Cu) nitrogen dioxide (NO2)2 ammonia (NH3)2, which is a copper based compound that converts from green to purple at 35° C. The intelligent ink may also include Bis (N,N-diethylethylenediamine)copper(II)perchlorate, which reversibly changes color from red to deep blue-purple at 43° C. The combination of CU(NO2)2(NH3)2 and Bis (N,N-diethylethylenediamine)copper(II)perchlorate establishes a temperature window of 35° C. to 43° C., the departure from which may be visible by dramatic color change. These compounds may be robustly encapsulated in polymer microspheres to avoid chemical interaction with the in-animal environment.

An intelligent ink that is responsive to glucose may include a color changing marker for glucose that can be injected into the skin of an animal. The color changing marker for glucose may have good correlation to blood glucose measurements taken using a glucometer.

One such marker or intelligent ink in an animal tattoo may operate as follows. The intelligent ink of the tattoo may contain nanospheres that contain a covalently bound phenylboronic acid derivative as well as two attached fluorophores that have been synthesized. The nanospheres may vary in size dependent on sugar concentration. In the absence of glucose the nanospheres may be small and may thereby provide a relatively dense or concentrated color for the tattoo. As glucose concentration increases, the glucose may bond with the acid and may increase the size of the fluorophores, which may in turn decrease the color density or color concentration of the tattoo, or at least of the part of the tattoo that includes the intelligent ink.

Another such marker or intelligent ink may target hydration level of the animal's blood by responding to electrolytes in the blood. For example, a responsive dye used in the intelligent ink may be encapsulated within microspheres, such as 120 micron microspheres. The microspheres may be coated with a biocompatible material and may be implanted in the subcutis of the animal as the intelligent ink that makes up all or a part of the tattoo. The microspheres may be configured to be porous to small cations. Cations from interstitial space may migrate into the microspheres such that the concentration within the microspheres and within interstitial space is the same. The presence of cations near the dye may cause the dye to lose an electron and become fluorescent. The relative fluorescence of the portion of the tattoo that includes the intelligent ink made up of these microspheres can be used to determine sodium concentration within interstitial space, from which hydration level can be inferred.

Other intelligent inks may alternately or additionally be implemented for animal lifecycle monitoring as described herein.

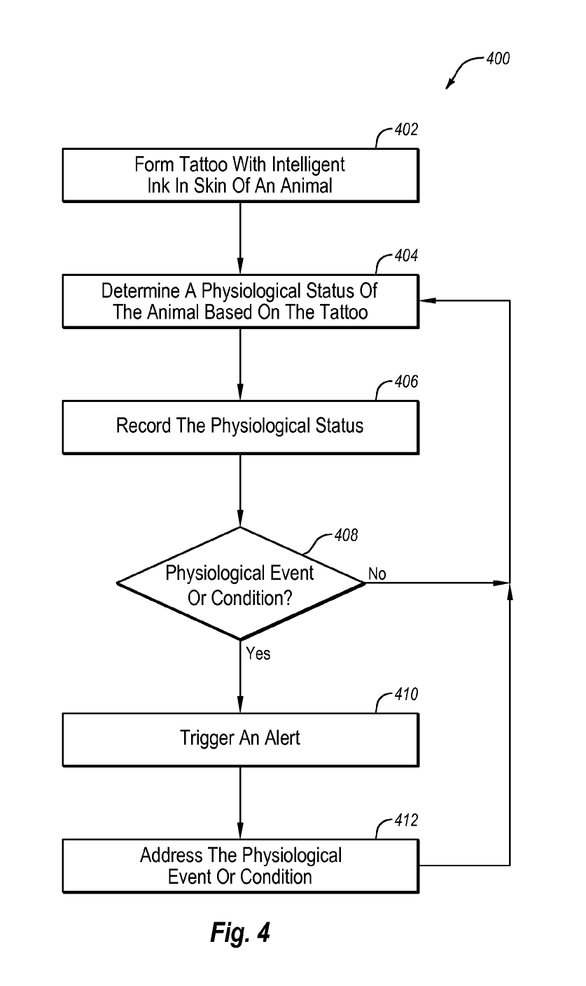

FIG. 4 is a flowchart of an example method 400 to monitor an animal, arranged in accordance with at least some embodiments described herein. The method 400 may be implemented, in whole or in part, by the system 100A of FIG. 1A, the system 100B of FIG. 1B, or another suitable system. For this and other processes and methods disclosed herein, the operations performed in the processes and methods may be implemented in differing order. Furthermore, the outlined operations are only provided as examples, and some of the operations may be optional, combined into fewer operations, supplemented with other operations, or expanded into additional operations without detracting from the essence of the disclosed embodiments. The method 400 includes various operations, functions, or actions as illustrated by one or more of blocks 402, 404, 406, 408, 410, and/or 412. The method 400 may begin at block 402.

In block 402 [“Form Tattoo With Intelligent Ink In Skin Of An Animal”], a tattoo with intelligent ink may be formed in the skin of an animal. The tattoo may be formed by injection of the intelligent ink into the skin of the animal using, e.g., the tattoo applicator 110 of FIGS. 1A-1B and/or the tattoo applicator 304 of FIG. 3A. Alternatively, the tattoo may be formed by applying the intelligent ink directly to a surface of the skin and solvating the intelligent ink into the skin. The intelligent ink may be injected or solvated into any desired layer of the animal's skin. Block 402 may be followed by block 404.

In block 404 [“Determine A Physiological Status Of The Animal Based On The Tattoo”], a physiological status of the animal may be determined based on the tattoo. The physiological status may be determined by, e.g., the health monitor application 112 and/or the tattoo reader 106 of FIGS. 1A-1B. Determining the physiological status of the animal based on the tattoo that includes the intelligent ink may include receiving light reflected, transmitted, emitted, and/or interfered by the intelligent ink; determining a current value of an optical property of the intelligent ink based on the received light; and comparing the current value of the optical property to a baseline value. The baseline value may include a current value of an optical property of a non-intelligent ink used as a baseline and/or static reference. Alternatively or additionally, the baseline value may include a previous value of the optical property of the intelligent ink, one or more possible values associated with the intelligent ink, or a spectrum of values associated with the intelligent ink. In these and other embodiments, determining the physiological status of the animal based on the tattoo that includes the intelligent ink may further include illuminating the intelligent ink with UV light. Determining the current value of the optical property of the intelligent ink based on the received light may include determining an optical property associated with fluorescence emitted by the intelligent ink in response to absorption by the intelligent ink of at least some of the UV light.

The current value, the previous value, the baseline value, and/or other values of optical properties may be determined using, e.g., the tattoo reader 106 of FIGS. 1A and 1B and/or by naked eye observation. The current value, the previous value, the baseline value, and/or other values of optical properties may include quantitative values such as a wavelength or wavelength range (e.g., 450 to 495 nm or 620 to 750 nm) and/or qualitative values such as a color description (e.g., blue or red).

Alternately or additionally, determining the physiological status of the animal based on the tattoo that includes the intelligent ink may include determining that the physiological status of the animal with respect to a particular target of physiology to which the intelligent ink is configured to respond is normal or indicative of a particular physiological event or condition. Such a determination may be made, e.g., in response to the current value of the optical property being within a particular range of the baseline value. Alternately, determining the physiological status of the animal based on the tattoo that includes the intelligent ink may include determining that the physiological status of the animal with respect to the particular target is abnormal or indicative of a particular physiological event or condition. Such a determination may be made, e.g., in response to the current value of the optical property being outside the particular range of the baseline value. Block 404 may be followed by block 406.

In block 406 [“Record The Physiological Status”], the physiological status of the animal may be recorded. The physiological status may be recorded by, e.g., the health monitor application 112 of FIGS. 1A-1B and/or manually by a person. The physiological status may be recorded in the animal records 114 of FIGS. 1A-1B. Recording the physiological status may include recording a measurement of the optical property of the intelligent ink, such as a numerical/quantitative value or a qualitative value of the measured optical property. Alternately or additionally, recording the physiological status may include recording an interpretation of the measurement of the optical property, recording a timestamp or another indicator of when the measurement is received from the tattoo reader and/or entered into the animal records, or recording some other information. The physiological status may generally be used any time after it is determined in block 404 and/or after it is recorded in block 406 to inform how the animal is managed and/or cared for. Block 406 may be followed by block 408.

In block 408 [“Physiological Event Or Condition?”], it is determined whether the physiological status indicates a physiological event or condition. The determination of whether the physiological status indicates a physiological event or condition may be made by, e.g., the health monitor application 112 of FIGS. 1A-1B. The physiological event or condition can include stress, pregnancy, fever, hypoglycemia, hyperglycemia, dehydration, infection by one or more pathogens, the absence of any of the foregoing, or other physiological event or condition. Block 408 may be followed by block 404 (“No” at block 408) or by block 410 (“Yes” at block 408).

In block 410, and in response to the physiological status indicating a physiological event or condition, an alert may be triggered to address the physiological event or condition. Triggering an alert to address the physiological event or condition may include generating an electronic message that identifies the animal and the physiological event or condition and/or sending the electronic message to a person, such as a farmer, farmhand, veterinarian, a healthcare provider, or other person. Alternately or additionally, the electronic message may include a recommended treatment to address the physiological event or condition. The alert may be triggered by, e.g., the health monitor application 112 of FIGS. 1A-1B. The electronic message may include a text message (e.g., SMS or Short Message Service), a multimedia message service (MMS) message, an email, an instant message (IM), a chat, or other suitable electronic message. Block 410 may be followed by block 412.

In block 412, the physiological event or condition may be addressed. For example, if the animal is being raised for slaughter and the physiological status indicates that the animal's blood glucose level is such that the animal is likely to be dark cutting, the physiological event or condition may be addressed by changing the animal's diet, keeping the animal at a farm until its glycogen levels are back to normal, or in some other manner. As another example, if the animal is a breeding animal and the physiological status indicates that the animal is pregnant, the physiological event or condition may be addressed by properly managing the animal in light of the pregnancy. As yet another example, if the animal is a dairy cow and the physiological status indicates that the animal has a pathogen or other physiological event or condition, a veterinarian may treat the animal for the pathogen or other physiological event or condition and/or other action may be taken to address the physiological event or condition. Block 412 may be followed by block 404.

The method 400 has been discussed in the context of a single intelligent ink. Alternately or additionally, the tattoo may include multiple intelligent inks injected into the skin of the animal Each of the intelligent inks may depend on a different one of multiple targets of physiology of the animal. In these and other embodiments, determining the physiological status of the animal based on the tattoo that includes the intelligent into at block 404 may include determining the physiological status of the animal with respect to each of the targets of physiology of the animal.

Blocks 404, 406, 408, 410, and/or 412 may be performed one time or may be repeated on a regular, periodic, and/or random schedule to monitor the physiological status of the animal throughout all or a portion of its lifecycle.

Some embodiments disclosed herein include a non-transitory computer-readable medium that includes computer-readable instructions stored thereon. In response to execution by a processor, the computer-readable instructions may cause the processor to perform or may cause the processor to control performance of the method 400 and/or variations thereof. In these and other embodiments, the processor may be included in a server, tattoo reader, or computing device, such as the server 104, the tattoo reader 106, and the computing device 120 of FIGS. 1A-1B.

The embodiments described herein may find application in a variety of areas. For example, tattoos with intelligent inks may be used for meat quality monitoring in animals raised for slaughter. In more detail, multiple measurements over time of an animal's tattoo with at least one intelligent ink may be used to develop an indication of a metabolic profile of the animal. Higher blood glucose levels may be a useful, non-invasive indicator of the animal consuming muscle or liver glycogen, indicating the animal may be dark cutting.

As implemented for multiple animals on a farm, the animals may be scanned to determine a likelihood that each is dark cutting. Those with a relatively low likelihood may be taken to slaughter. Those with a relatively higher likelihood may be left on the farm for a carbohydrate loading protocol before being taken to slaughter. Thus, a number of dark cutting animals may be reduced.

Tattoos with intelligent inks may alternately or additionally be used to monitor dairy animals. Monitoring may be done on a day-to-day basis as each animal is milked. The tattoo may be applied to the udder of the animal or other location of the animal. A tattoo reader, such as the tattoo reader 106 of FIGS. 1A-1B, may be integrated into or placed in close proximity with each milk station. As each animal is prepared for milking, the tattoo may be read by the tattoo reader automatically and/or by a person scanning the tattoo reader across the tattoo. The physiological status of the animal may be recorded in a corresponding animal record to provide a daily (or other period) physiological status across the animal's lifetime.

As another example, tattoos with intelligent inks may alternately or additionally be used to monitor breeding animals. The heath status of each animal in a breeding program may be monitored as described herein upon entry into the breeding program and throughout the breeding program. The tattoos may allow the physiological status to be monitored non-invasively.

Other examples described elsewhere herein include use of tattoos with intelligent inks in endangered species, pets, and humans.

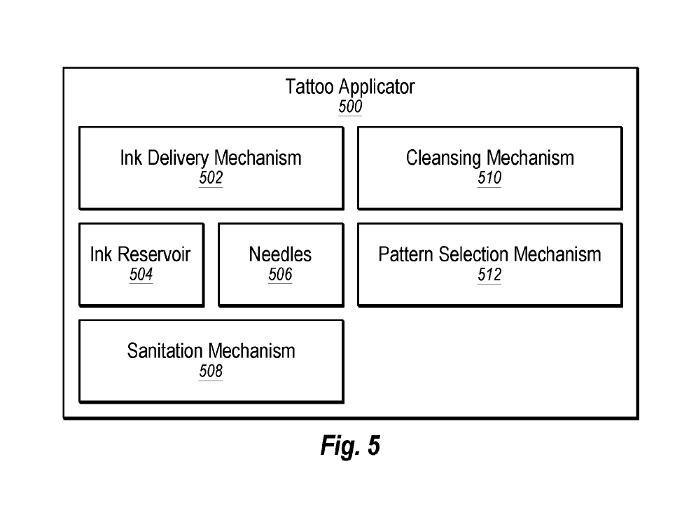

FIG. 5 is a block diagram of an example tattoo applicator 500, arranged in accordance with at least some embodiments described herein. The tattoo applicator 500 may be configured to apply a tattoo to skin of a human, an animal, or other tattoo recipient. The tattoo applicator 500 may include an ink delivery mechanism 502 and multiple needles 506. In some embodiments, the tattoo applicator 500 may include a sanitation mechanism 508, a cleansing mechanism 510, and/or a pattern selection mechanism 512.

The needles 506 may include any suitable system, apparatus, or device that may be configured to inject ink, agents, or other ink elements into or onto skin to form a tattoo. In some embodiments, the needles 506 may be manufactured using any suitable or existing tattoo or medical needle manufacturing technique. The needles 506 may be selected and/or manufactured to have a durability that allows for multiple uses. For example, the needles 506 may have a durability that allows for the needles 506 to be used between approximately 25 and 150 times. In other examples, the needles 506 may include self-dissolving, skin-absorbable materials that are used once and left in the skin to be dissolved and absorbed.

The needles 506 may be configured to inject the ink into a dermis of the skin such as is commonly performed in applying tattoos. Alternately or additionally, the needles 506 may be configured to inject the ink into a subcutis or other layer of the skin. In some embodiments, the needles 506 may include needles of different lengths to deliver different ink elements to different layers in the skin.

In some embodiments the ink may include an intelligent ink and the needles 506 may be configured to inject the intelligent ink into the subcutis of the skin of an animal. The intelligent ink may include an ink that responds to a particular target of physiology of the animal and the injection of the intelligent ink into the subcutis may allow for the intelligent ink to interact with the blood of the animal. The intelligent ink may be configured to change color or another optical property dependent on the particular target of physiology. The particular target of physiology may be indicative of a physiological status of the animal. Accordingly, the optical property of the intelligent ink may be measured to determine the physiological status of the animal In some embodiments, if the physiological status indicates a particular physiological event or condition, an alert may be triggered to address the physiological event or condition. Examples of various intelligent inks and usage thereof are described elsewhere herein.

Alternatively or additionally, the ink may include an intelligent ink, a depigmentation agent, and/or a depilatory agent. Some of the needles 506 may have one length and may be configured to deliver the intelligent ink to one layer or depth within the skin. Others of the needles 506 may have another length and may be configured to deliver the depigmentation agent to another layer or depth within the skin. Still others of the needles 506 may have yet another length and may be configured to deliver the depilatory agent to yet another layer or depth within the skin.

In some embodiments, at least a subset of the needles 506 may be configured to inject the ink at substantially the same time such that a substantial portion of the tattoo—or, in some embodiments, the entire tattoo—may be applied at substantially the same time. As explained further below, in some embodiments the ink delivery mechanism 502 and the needles 506 may be configured such that the needles 506 may inject different types of ink or different elements of the ink (e.g., different intelligent inks and/or different agents) into different portions of an area of the skin and/or to different layers or substructures of the skin that is tattooed to achieve desired properties of the tattoo. For example, the ink delivery mechanism 502 and the needles 506 may be configured such that different types of intelligent ink may be tattooed to different portions of the skin of an animal. The different types of intelligent ink may provide information related to different blood properties or animal properties. Therefore, different blood properties or other properties of the animal may be monitored by monitoring the different portions of the skin that include the different types of intelligent ink.

In some embodiments, the needles 506 may be configured in a relatively dense pattern (e.g., between 10-600 needles per square centimeter). The density of the needles 506 may allow for a more thorough and even distribution of ink as compared to when a lower density is used. Improved distribution may improve the utility of some applications of tattoos such as tattoos that include intelligent ink.

The size of each of the needles 506 may be selected to be small enough to allow for a relatively high density of the needles 506 but to also provide enough durability to allow for repeated use of the needles 506. For example, the needles 506 may be made using materials commonly used for tattooing needles. In this particular example, the size of the needles 506 that may allow for a desired density and durability may be such that the needles 506 have a density between approximately 10-600 needles per square centimeter when grouped as close together as possible. The above sizes and corresponding density range of the needles 506 are merely examples. The sizes of the needles 506 may decrease and the corresponding density range of the needles 506 may increase depending on the materials used for the needles 506 and their associated durability. Alternatively or additionally, one or more of the needles 506 may be made using self-dissolving, skin-absorbable materials such as dextrin, chondroitin sulphate, albumin, sodium chloride, polymers, or other substances.

The needles 506 may be configured according to a pattern such that the tattoo may be formed with the desired pattern when the needles 506 inject the ink into the skin. The pattern may include a simple shape, a complex shape, a number, a letter, a picture, a barcode, or any other suitable pattern. In some embodiments, the pattern may be based on a desired purpose of the tattoo. For example, when the tattoo is used for identification of an animal, the pattern may include a number, a letter, and/or a barcode that may be used to identify the animal. As another example, when the ink includes intelligent ink, the desired pattern may include a shape where the intelligent ink is densely embedded in the skin. In these or other embodiments, the tattoo may have more than one use. For example, an identification tattoo may also include intelligent ink such that the tattoo may be used to identify an animal and to monitor its physiological status.

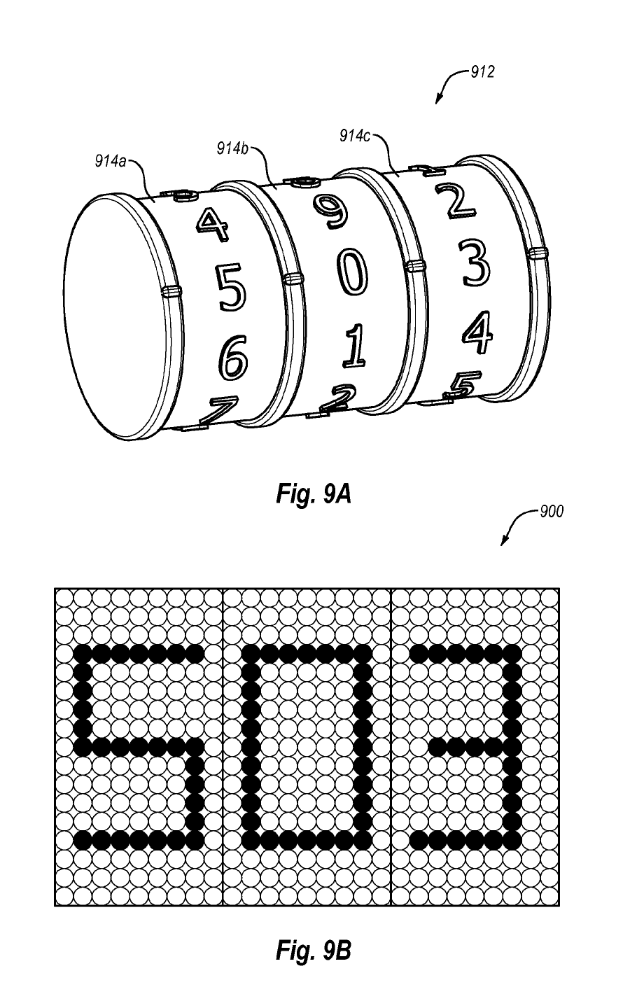

In these or other embodiments, the pattern of the needles 506 may be changed using the pattern selection mechanism 512. The pattern selection mechanism 512 may include any suitable system, apparatus, or device that may be configured to change the pattern of the needles 506 that may inject the ink into the skin. Accordingly, the pattern selection mechanism 512 may change the pattern of the corresponding tattoo. An example pattern selection mechanism 512 is described below with respect to FIG. 9A.

In some embodiments, one or more of the needles 506 may be perforated and may be referred to as “perforated needles.” The perforated needles may be perforated along at least a portion of the respective shafts of the perforated needles such that at least a portion of the shafts of the perforated needles may be hollow. The perforation may extend to a tip portion of each of the perforated needles and may be configured to carry ink and/or a sanitation agent to the tip portion of the perforated needles.

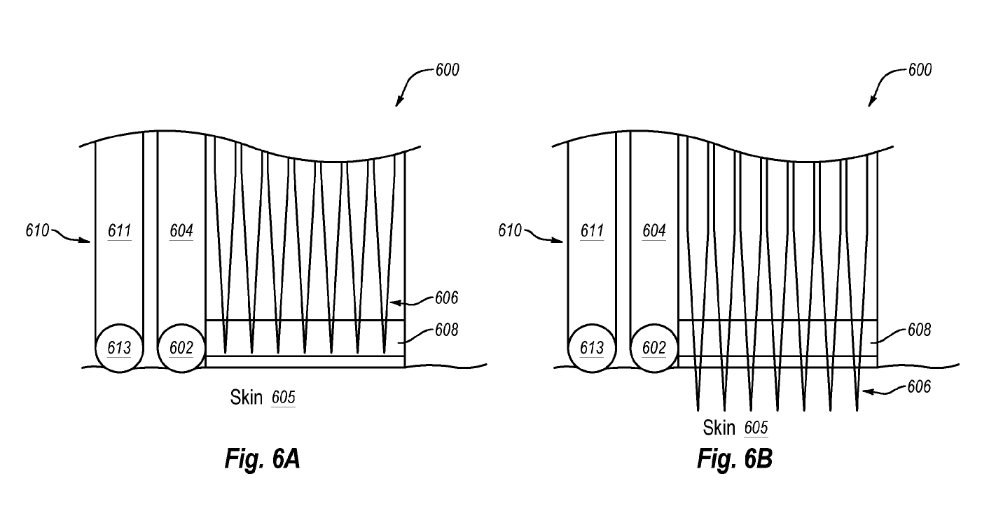

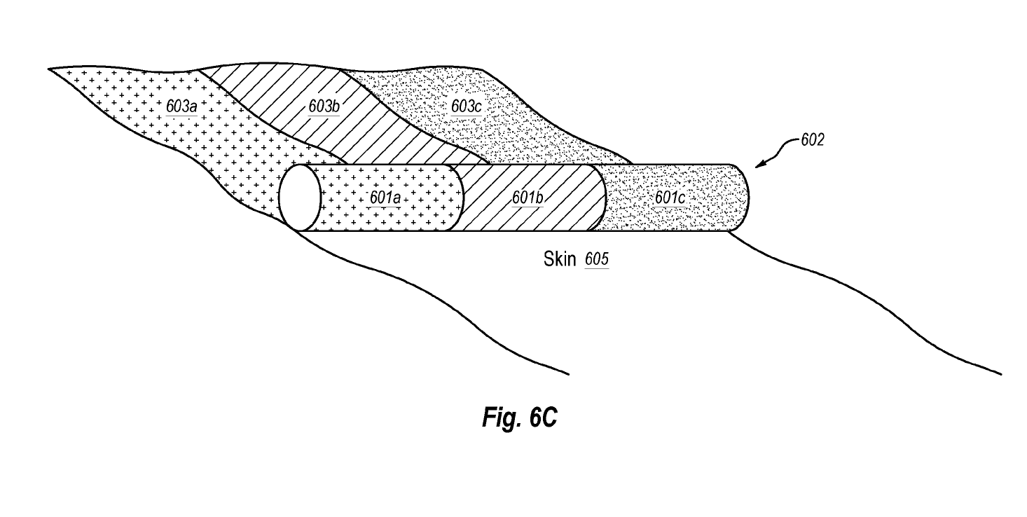

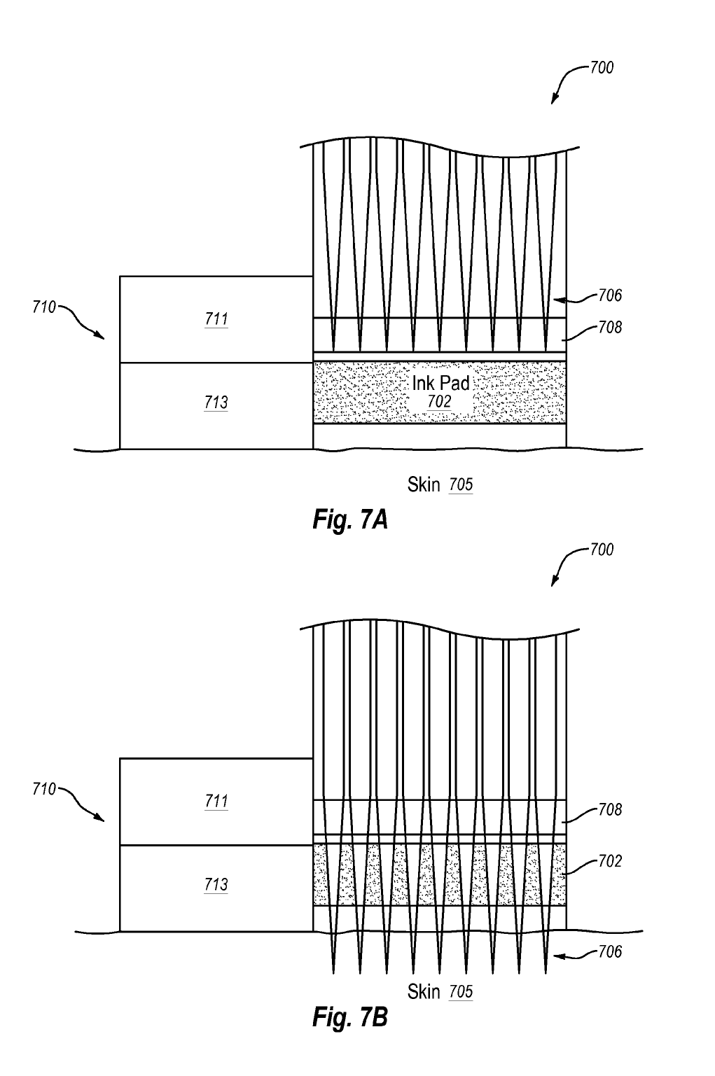

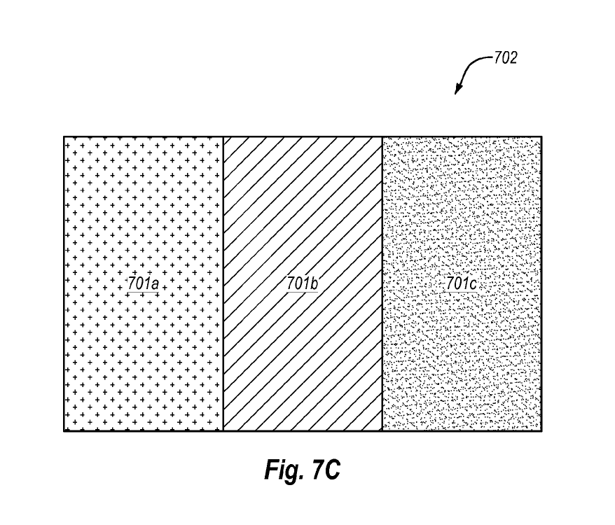

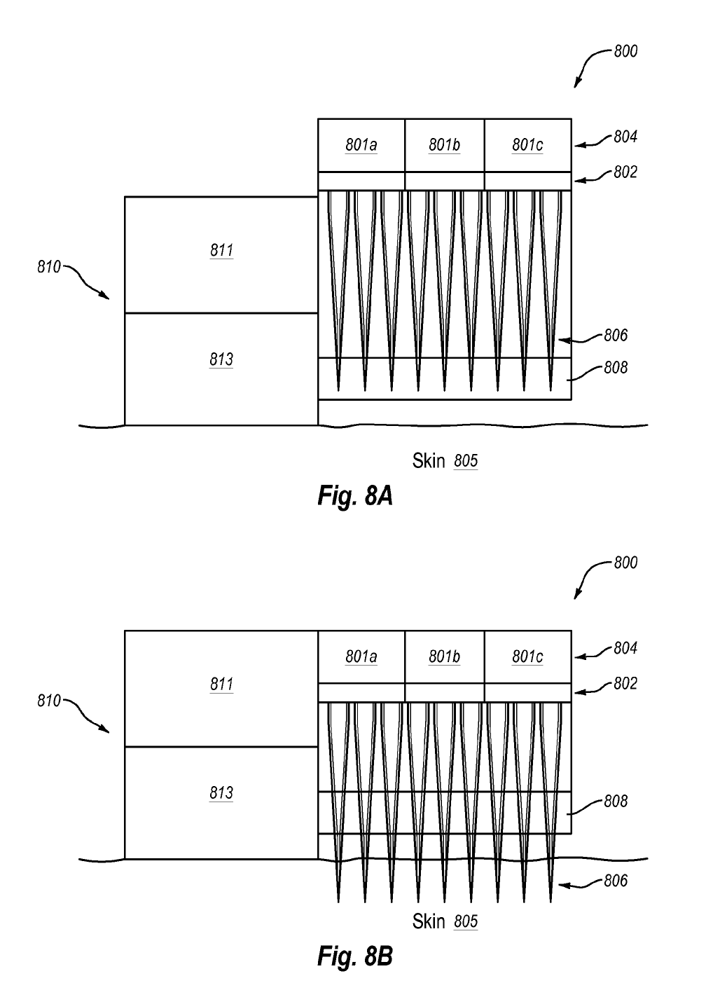

The ink delivery mechanism 502 may include any suitable system, apparatus, or device configured to deliver ink to the needles 506 such that the needles 506 may inject the ink into any layer or substructure of skin and/or may deliver ink to the surface of the skin. For example, in some embodiments, the ink delivery mechanism 502 may include one or more rollers that are configured to roll ink onto an area of the skin at an epidermal layer of the skin. The tattoo applicator 500 may be configured to activate the needles 506 after the ink has been delivered onto the area of the skin such that the needles 506 inject the ink from the epidermis to the dermis and/or subcutis of the skin. An example tattoo applicator that includes a roller is described below with respect to FIGS. 6A-6C. As another example, the ink delivery mechanism 502 may include an ink pad configured such that the needles 506 may pass through the ink pad prior to puncturing the skin. The needles 506 may be configured to retain some ink on their tips from passing through the ink pad such that the retained ink may be injected into the skin. An example tattoo applicator that includes an ink pad is described below with respect to FIGS. 7A-7C. As another example, when the needles 506 include perforated needles, the ink delivery mechanism 502 may be configured to divert ink to the tips of the perforated needles through the hollow portions of the perforated needles. An example tattoo applicator that delivers ink through the hollow portion of perforated needles is described below with respect to FIGS. 8A and 8B.

In some embodiments, the tattoo applicator 500 may include an ink reservoir 504 configured to retain ink. The ink reservoir 504 may be configured to provide ink that may be delivered by the ink delivery mechanism 502. For example, when the ink delivery mechanism 502 includes a roller, the ink reservoir 504 may be configured to supply ink to the roller. As another example, when the ink delivery mechanism 502 includes an ink pad, the ink reservoir 504 may be configured to provide ink to the ink pad. Further, when the ink delivery mechanism 502 is configured to deliver ink to the hollow portions of perforated needles, the ink reservoir 504 may be configured to retain the ink that may be delivered to the hollow portions. In these or other embodiments, the ink reservoir 504 may be partitioned such that different types of ink may be delivered to different portions of the ink delivery mechanism 502 and accordingly to different needles, to different portions of the skin that is tattooed, and/or to different layers or skin substructures of the skin that is tattooed.

As indicated above, the tattoo applicator 500 may also include a sanitation mechanism 508. The sanitation mechanism 508 may be configured to apply a sanitation agent to the needles 506 between applications of tattoos. For example, in some embodiments, the sanitation mechanism 508 may include a sponge that may be saturated in the sanitation agent. Further, the needles 506 may be configured to puncture the skin and then retract into the tattoo applicator 500. In these or other embodiments, when the needles 506 are retracted, they may be configured to sit within the sponge such that the sanitation agent of the sponge may be in contact with the needles 506. Examples of such a configuration are given with respect to FIGS. 6A-6B, 7A-7B, and 8A-8B. In these or other embodiments, such as when the needles 506 are perforated, the sanitation mechanism may be configured to route the sanitation agent through the hollow portion of the perforated needles to the tips of the perforated needles. The sanitation agent may include any suitable agent that may be configured to kill a substantial number of germs that may be on the needles 506. For example, ethanol may be used as the sanitation agent.

Additionally, in some embodiments, the tattoo applicator 500 may include the cleansing mechanism 510. The cleansing mechanism 510 may include any suitable system, apparatus, or device configured to cleanse the area of the skin to which a tattoo may be applied by the tattoo applicator 500. For example, in some embodiments, the cleansing mechanism 510 may include a sponge that is saturated in a cleansing agent. In some embodiments, the cleansing agent may include a sanitation agent that may be the same as or different from the sanitation agent that may be used with the sanitation mechanism 508.

Modifications, additions, or omissions may be made to the tattoo applicator 500 without departing from the scope of the present disclosure. For example, the tattoo applicator 500 may include other elements than those specifically described. Further, the relationship between elements of the tattoo applicator may vary depending on specific configurations. Additionally, the configuration may vary depending on the desired application. For example, in some embodiments, the tattoo applicator 500 may be configured in a pliers-like configuration such that one or more elements of the tattoo applicator 500 may interact with both sides of an ear of an animal to which the tattoo is to be ablied.

FIGS. 6A and 6B illustrate an example configuration of some elements of a tattoo applicator 600, arranged in accordance with at least some embodiments described herein. The tattoo applicator 600 may be configured to apply a tattoo to skin 605.

The tattoo applicator 600 may include an ink roller 602 and an ink reservoir 604, which may be examples of the ink delivery mechanism 502 and the ink reservoir 504, respectively, of FIG. 5. The ink roller 602 may be configured to apply ink to the skin 605 in a rolling motion. The ink reservoir 604 may be configured to supply ink to the ink roller 602. In some embodiments, the ink roller 602 and the ink reservoir 604 may be configured such that different types of ink may be applied to different portions of an area of the skin 605 that is to be tattooed.

For example, FIG. 6C illustrates an example where the ink roller 602 and the ink reservoir 604 are configured to apply different types of ink to different portions of the skin 605, according to at least some embodiments described herein. In the illustrated embodiment of FIG. 6C, the ink reservoir 604 (not depicted in FIG. 6C) may be partitioned such that one ink, another ink, and yet another ink are applied to one portion 601a, another portion 601b, and yet another portion 601c, respectively, of the ink roller 602. Accordingly, when the ink roller 602 rolls across the skin 605, the portion 601a of the ink roller 602 may apply one ink to a portion 603a of the skin 605, the portion 601b of the ink roller 602 may apply another ink to a portion 603b of the skin 605, and the portion 601c of the ink roller 602 may apply yet another ink to a portion 603c of the skin 605. The different inks may each have different properties such that they may be different ink types and/or different ink elements. For example, the different inks may include different types of intelligent ink that may provide information related to different targets of physiology or the different inks may merely include inks of different colors. Therefore, different ink types may be applied to different portions of the skin 605 such that when ink is injected into the skin 605 (as described below), ink of different types may be injected into the portion 603a, the portion 603b, and the portion 603c of the skin 605.

The tattoo applicator 600 may include multiple needles 606 that may be examples of the needles 506 described with respect to FIG. 5. FIG. 6A illustrates the needles 606 in a retracted state and FIG. 6B illustrates the needles 606 in an unretracted state in which the needles 606 have punctured the skin 605. The needles 606 may be configured to transition from the retracted state to the unretracted state over an area of the skin 605 that has been passed over by the ink roller 602. Therefore, the needles 606 may be configured to inject the ink from a surface of an epidermis of the skin 605 to a dermis or subcutis of the skin 605 when transitioning from the retracted state to the unretracted state. Additionally, by applying the ink to the skin 605 in a manner that allows the needles 606 to inject the ink into the skin, the needles 606 and the ink roller 602 may interact in a manner in which the ink roller 602 delivers ink to the needles 606.

The needles 606 may be configured to retract and unretract using any suitable system, apparatus, or device. For example, in some embodiments, the needles 606 may be configured such that when they are in the refracted state they are spring loaded. In some of these embodiments, the tattoo applicator 600 may include a triggering mechanism (not illustrated) that may release the spring (not illustrated) in response to a user input in a manner that causes the needles 606 to move to the unretracted state. In these or other embodiments, the needles 606 may be reset to the retracted state through a cocking motion and mechanism that may reload the needles 606 into the spring loaded retracted state.

Further, in some embodiments, the needles 606 may be configured with the ink roller 602 such that after the ink roller 602 has rolled a certain distance in a direction moving away from the needles 606, the needles 606 may transition from the retracted state to the unretracted state. For example, a gearing mechanism may be configured between the ink roller 602 and the needles 606 to move the needles 606 between the refracted and unretracted states as the ink roller 602 rolls in the direction from the needles 606 toward the ink roller 602. As another example, the ink roller 602 may be configured to release the spring after the ink roller 602 has rolled a certain distance in configurations when the needles 606 are spring loaded.

In some embodiments, the tattoo applicator 600 may include a sanitation sponge 608 that may be soaked in a sanitation agent. The sanitation sponge 608 may be an example of the sanitation mechanism 508 of FIG. 5. As illustrated in FIG. 6A, when the needles 606 are in the retracted state, tips of the needles 606 may rest substantially inside of the sanitation sponge 608. Therefore, the tips of the needles 606 may be exposed to the sanitation agent. As illustrated in FIG. 6B, at least a portion of the needles 606 may be configured to pass through the sanitation sponge 608 when the needles 606 transition from the retracted state to the unretracted state.

The tattoo applicator 600 may also include a cleansing mechanism 610, which may include a cleansing roller 613 and a cleansing agent reservoir 611. The cleansing mechanism 610 may be an example of the cleansing mechanism 510 of FIG. 5. The cleansing roller 613 may be configured to apply a cleansing agent to the skin 605 prior to the skin 605 being tattooed. For example, the tattoo applicator 600 may be configured such that it may be moved across the skin 605 in the direction moving from the needles 606 to the ink roller 602 and the cleansing roller 613. Therefore, the cleansing roller 613 may pass over the skin 605 before the ink roller 602 and before the needles 606 inject the ink into the skin 605. The cleansing agent may be provided to the cleansing roller 613 by the cleansing agent reservoir 611. As indicated above, the cleansing agent may include a sanitation agent that may be the same as or different from the sanitation agent in which the sanitation sponge 608 may be soaked.

In FIGS. 6A-6B, the needles 606 are illustrated as having a same length and penetrating to a same layer or depth of the skin 605. In other embodiments, the needles 606 may have different lengths and/or may penetrate to different layers or depths of the skin 605. For example, some of the needles 606 may have one length and may penetrate to one layer or depth while others of the needles 606 may have another length and may penetrate to a different layer or depth. Alternately or additionally, the needles 606 that penetrate to one layer or depth may deliver one intelligent ink or agent, while the needles 606 that penetrate to another layer or depth may deliver a different intelligent ink or agent. Alternately or additionally, the needles 606 may have three or more different lengths and they may deliver three or more different intelligent inks or agents to three or more different layers or depths of the skin 605.

Modifications, additions, or omissions may be made to the tattoo applicator 600 without departing from the scope of the present disclosure. For example, the illustrated embodiment is merely to provide a conceptual illustration of how different components of the tattoo applicator 600 may interact with each other. The actual configuration and interaction with different components may vary. Additionally, any number of other components may be present to help facilitate the functionality described herein.

FIGS. 7A and 7B illustrate an example configuration of some elements of a tattoo applicator 700, arranged in accordance with at least some embodiments described herein. The tattoo applicator 700 may be configured to apply a tattoo to an area of skin 705 as described below.

The tattoo applicator 700 may include an ink pad 702, which may be an example of the ink delivery mechanism 502 of FIG. 5. In some embodiments, the tattoo applicator 700 may also include an ink reservoir (not illustrated) that may be configured to provide ink to the ink pad 702. In some embodiments, the ink pad 702 may include different types of ink such that different types of ink may be tattooed to different portions of the area of the skin 705 that may be tattooed.

For example, FIG. 7C illustrates an example of the ink pad 702 including a portion 701a, a portion 701b, and a portion 701c, arranged in accordance with at least some embodiments described herein. The portion 701a may include one ink saturated therewith, the portion 701b may include another ink saturated therewith, and the portion 701c may include yet another ink saturated therewith. The different inks may have one or more different properties such that they may be of different types. Accordingly, when ink from the ink pad 702 is injected into the skin 705 (as explained below) different portions of an area of the skin 705 that may be tattooed may be injected with different types of ink.

The tattoo applicator 700 may include multiple needles 706 that may be examples of the needles 506 described with respect to FIG. 5. FIG. 7A illustrates the needles 706 in a retracted state and FIG. 7B illustrates the needles 706 in an unretracted state in which the needles 706 have punctured the skin 705. As illustrated in FIGS. 7A and 7B, the needles 706 may be configured to pass through the ink pad 702 when the needles 706 transition from the retracted state to the unretracted state. Additionally, the needles 706 may be configured such that when the needles 706 pass through the ink pad 702, the needles 706 may retain some ink on the tips of the needles 706. Therefore, when the needles 706 puncture the skin 705, the needles may inject the ink into a dermis or subcutis of the skin 705 when transitioning from the retracted state to the unretracted state.

The needles 706 may be configured to retract and unretract using any suitable system, apparatus, or device. For example, in some embodiments, the needles 706 may be configured such that when they are in the retracted state they are spring loaded. In some of these embodiments, the tattoo applicator 700 may include a triggering mechanism (not illustrated) that may release the spring (not illustrated) in response to a user input in a manner that causes the needles 706 to transition to the unretracted state. In these or other embodiments, the needles 706 may be reset to the retracted state through a cocking motion and mechanism that may reload the needles 706 into the spring loaded retracted state.

In some embodiments, the tattoo applicator 700 may include a sanitation sponge 708 that may be soaked in a sanitation agent. The sanitation sponge 708 may be an example of the sanitation mechanism 508 of FIG. 5. As illustrated in FIG. 7A, when the needles 706 are in the retracted state, tips of the needles 706 may rest substantially inside of the sanitation sponge 708. Therefore, the tips of the needles 706 may be exposed to the sanitation agent. As illustrated in FIG. 7B, at least a portion of the needles 706 may be configured to pass through the sanitation sponge 708 when the needles 706 transition from the retracted state to the unretracted state.

The tattoo applicator 700 may also include a cleansing mechanism 710, which may include a cleansing sponge 713 and a cleansing agent reservoir 711. The cleansing mechanism 710 may be an example of the cleansing mechanism 510 of FIG. 5. The cleansing sponge 713 may be configured to apply a cleansing agent to the skin 705 prior to the skin 705 being tattooed. For example, the tattoo applicator 700 may be configured such that it may be moved across the skin 705 while the needles 706 are in the retracted state before transitioning to the unretracted state with respect to the skin 705. Therefore, the cleansing agent may be applied to the skin 705 before the needles 706 inject the ink into the skin 705. The cleansing agent may be provided to the cleansing sponge 713 by the cleansing agent reservoir 711. As indicated above, the cleansing agent may be a sanitation agent that may be the same as or different from the sanitation agent in which the sanitation sponge 708 may be soaked.

In FIGS. 7A-7B, the needles 706 are illustrated as having a same length and penetrating to a same layer or depth of the skin 705. In other embodiments, the needles 706 may have different lengths and/or may penetrate to different layers or depths of the skin 705. For example, some of the needles 706 may have one length and may penetrate to one layer or depth while others of the needles 706 may have another length and may penetrate to a different layer or depth. Alternately or additionally, the needles 706 that penetrate to one layer or depth may deliver one intelligent ink or agent, while the needles 706 that penetrate to another layer or depth may deliver a different intelligent ink or agent. Alternately or additionally, the needles 706 may have three or more different lengths and they may deliver three or more different intelligent inks or agents to three or more different layers or depths of the skin 705.

Modifications, additions, or omissions may be made to the tattoo applicator 700 without departing from the scope of the present disclosure. For example, the illustrated embodiment is merely to provide a conceptual illustration of how different components of the tattoo applicator 700 may interact with each other. The actual configuration and interaction with different components may vary. Additionally, any number of other components may be present to help facilitate the functionality described herein.

FIGS. 8A and 8B illustrate an example configuration of some elements of a tattoo applicator 800, arranged in accordance with at least some embodiments described herein. The tattoo applicator 800 may be configured to apply a tattoo to skin 805.

The tattoo applicator 800 may include multiple needles 806 that may be examples of the needles 506 described with respect to FIG. 5. FIG. 8A illustrates the needles 806 in a retracted state and FIG. 8B illustrates the needles 806 in an unretracted state in which the needles 806 have punctured an area of the skin 805. The needles 806 may be configured to retract and unretract using any suitable system, apparatus, or device. For example, in some embodiments, the needles 806 may be configured such that when they are in the retracted state they are spring loaded. In some of these embodiments, the tattoo applicator 800 may include a triggering mechanism (not illustrated) that may release the spring (not illustrated) in response to a user input in a manner that causes the needles 806 to move to the unretracted state. In these or other embodiments, the needles 806 may be reset to the retracted state through a cocking motion and mechanism that may reload the needles 806 into the spring loaded retracted state. Further, the needles 806 may be perforated such that the needles 806 may include a hollow portion that may extend to a tip of each of the needles 806.

The tattoo applicator 800 may include an ink diverter 802 and an ink reservoir 804, which may be examples of the ink delivery mechanism 502 and the ink reservoir 504, respectively, of FIG. 5. The ink diverter 802 may be configured to divert ink into the hollow portions of the needles 806 such that the ink may be delivered to the tips of the needles 806. Therefore, when the needles 806 puncture the skin 805, the needles 806 may inject the ink into a dermal or subcutaneous layer of the skin 805. The ink reservoir 804 may be configured to store and supply the ink that may be diverted by the ink diverter 802.

In some embodiments, the ink diverter 802 and the ink reservoir 804 may be configured such that different types of ink and/or agents may be diverted to different needles of the needles 806 such that different portions of the area of skin 805 may be tattooed by the needles 806 with different types of ink or agents and/or such that different types of ink or agents may be delivered to different layers or structures of the skin 805. For example, as illustrated in FIGS. 8A and 8B, the ink diverter 802 and the ink reservoir 804 may be partitioned according to a portion 801a, a portion 801b, and a portion 801c. The ink reservoir 804 may be configured to retain a one ink in the portion 801a such that the ink may be delivered to the ink diverter 802 within the portion 801a. The ink diverter 802 within the portion 801a may then divert the ink to one subset of the needles 806. The ink reservoir 804 may be configured to retain another ink in the portion 801b such that the ink may be delivered to the ink diverter 802 within the portion 801b. The ink diverter 802 within the portion 801b may then divert the ink to another subset of the needles 806 that may be different from the previously mentioned subset of the needles 806. Additionally, the ink reservoir 804 may be configured to retain yet another ink in the portion 801c such that the ink may be delivered to the ink diverter 802 within the portion 801c. The ink diverter 802 within the portion 801c may then divert the ink to yet another subset of the needles 806 that may be different from the two previously mentioned subsets of the needles 806. Accordingly, when the needles 806 puncture the skin 805, one subset of the needles 806 may inject one ink, another subset of the needles 806 may inject another ink, and yet another subset of the needles 806 may inject yet another ink. The different inks may have one or more different properties such that they may be of different types. Therefore, different portions of the area of the skin 805 that may be tattooed may be injected with different types of ink.

In some embodiments, the tattoo applicator 800 may include a sanitation sponge 808 analogous to the sanitation sponge 608 and the sanitation sponge 708 described above with respect to FIGS. 6A, 6B, 7A, and 7B. Further, the tattoo applicator 800 may also include a cleansing mechanism 810, which may include a cleansing sponge 813 and a cleansing agent reservoir 811. The cleansing mechanism 810, the cleansing sponge 813, and the cleansing agent reservoir 811 may be analogous to the cleansing mechanism 710, the cleansing sponge 713, and the cleansing agent reservoir 711, respectively, of FIGS. 7A and 7B.

In FIGS. 8A-8B, the needles 806 are illustrated as having a same length and penetrating to a same layer or depth of the skin 805. In other embodiments, the needles 806 may have different lengths and/or may penetrate to different layers or depths of the skin 805. For example, some of the needles 806 may have one length and may penetrate to one layer or depth while others of the needles 806 may have another length and may penetrate to a different layer or depth. Alternately or additionally, the needles 806 that penetrate to one layer or depth may deliver one intelligent ink or agent, while the needles 806 that penetrate to another layer or depth may deliver a different intelligent ink or agent. Alternately or additionally, the needles 806 may have three or more different lengths and they may deliver three or more different intelligent inks or agents to three or more different layers or depths of the skin 806.

Modifications, additions, or omissions may be made to the tattoo applicator 800 without departing from the scope of the present disclosure. For example, the illustrated embodiment is merely to provide a conceptual illustration of how different components of the tattoo applicator 800 may interact with each other. The actual configuration and interaction with different components may vary. Additionally, any number of other components may be present to help facilitate the functionality described herein.

FIG. 9A illustrates an example pattern selection mechanism 912 of a tattoo applicator, arranged in accordance with at least some embodiments described herein. The pattern selection mechanism 912 is an example of the pattern selection mechanism 512 of FIG. 5. Although not expressly described or illustrated with respect to FIGS. 6A-8B, the pattern selection mechanism 912 may be included with one or more of the tattoo applicator 600, the tattoo applicator 700, and the tattoo applicator 800.

In the illustrated embodiment, the pattern selection mechanism 912 may allow for selection of a three digit number. For example, the pattern selection mechanism 912 may include a selector 914a, a selector 914b, and a selector 914c. The selector 914a may be configured to allow for selection of one digit of the three digit number, the selector 914b may be configured to allow for selection of another digit of the three digit number, and the selector 914c may be configured to allow for selection of yet another digit of the three digit number.

The selectors 914 may be configured to interact with needles of the corresponding tattoo applicator such that a selected number may be tattooed into the skin. For example, when a number is selected for one of the selectors 914, a corresponding subset of the needles may be configured such that the needles of the corresponding subset that puncture the skin form the selected number. By way of example, FIG. 9A illustrates that the number “503” is selected and FIG. 9B illustrates a bottom-view of a tattoo applicator 900 that includes the pattern selection mechanism 912. As illustrated in FIG. 9B, needles of the tattoo applicator 900 may be configured to tattoo the number “503” into skin according to the selection of the number “503” on the pattern selection mechanism 912.

Modifications, additions, or omissions may be made to the pattern selection mechanism 912 without departing from the scope of the present disclosure. For example, the pattern selection mechanism 912 may be modified such that it may be integrated with the housing of the tattoo applicator 900. Additionally, other components may be included that provide for the interaction between selection of a number and selecting the corresponding needles.

Further, the illustrated embodiment is merely an example configuration and any number of other configurations may be used to select a pattern. For example, in some embodiments, the needles may be organized into dies of different patterns and the dies may be removable. As such, one or more dies having a desired pattern may be selected and placed into the corresponding tattoo applicator.

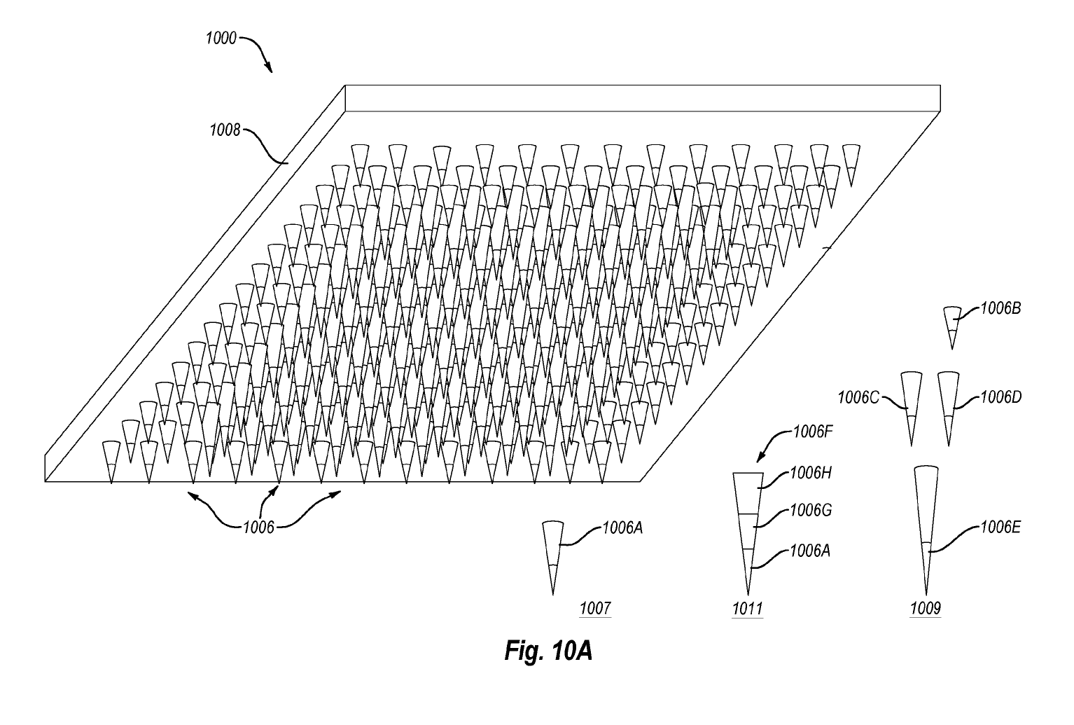

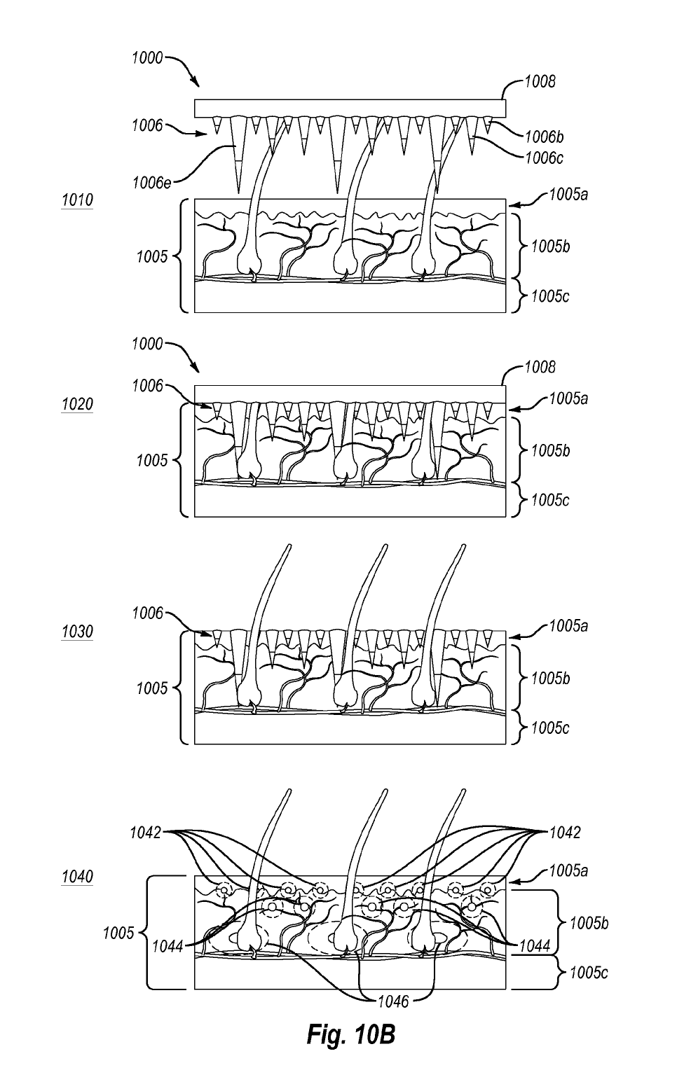

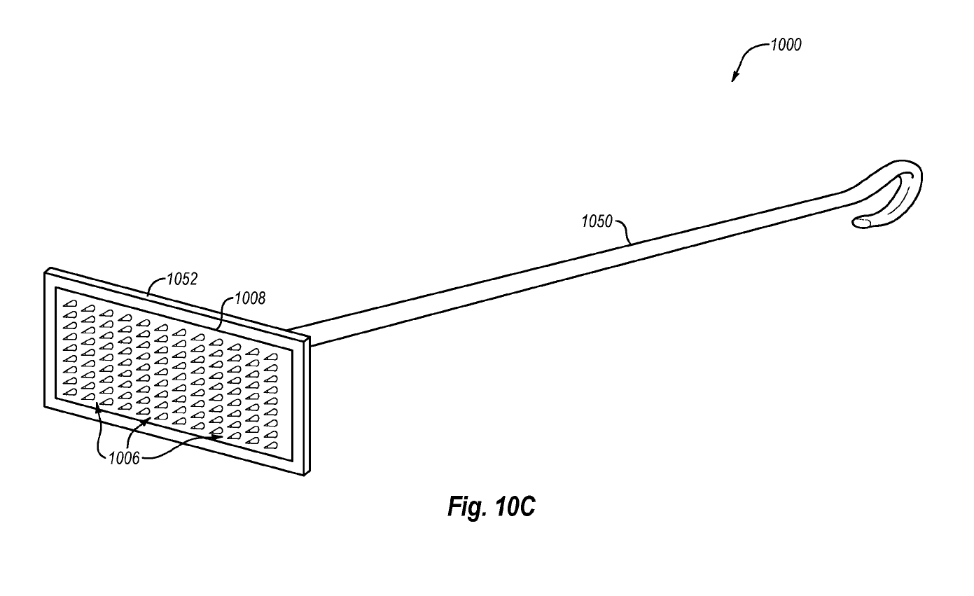

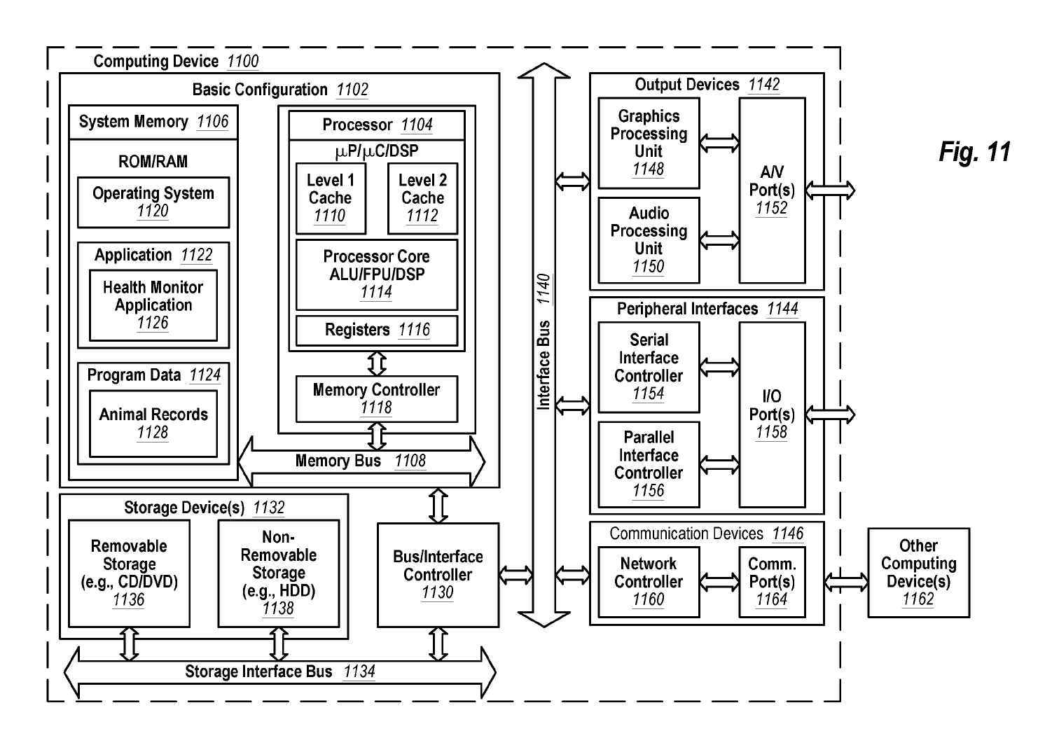

FIGS. 10A and 10B illustrate an example configuration of some elements of another tattoo applicator 1000, arranged in accordance with at least some embodiments described herein. The tattoo applicator may be configured to apply a tattoo to skin 1005 (FIG. 10B).

The tattoo applicator 1000 may include multiple microneedles 1006 that may be examples of the microneedles 506 described with respect to FIG. 5. FIG. 10A includes a detail view 1007 of one of the microneedles 1006. The microneedle 1006 in the detail view 1007 includes a self-dissolving and skin-absorbable material that forms a structure of the microneedle 1006. For example, the material may include dextrin, chondroitin sulphate, albumin, sodium chloride, one or more polymers, or other self-dissolving and skin-absorbable material(s). The microneedle 1006 in the detail view 1007 additionally includes a tip 1006A infused with an intelligent ink and/or an agent to aid in the visualisation of the intelligent ink. Each of the microneedles 1006 may generally include a similar configuration as illustrated in the detail view 1007.

In some embodiments, some of the microneedles 1006 of the tattoo applicator 1000 may include tips 1006A with the intelligent ink while others may include tips 1006A with the agent. Alternatively or additionally, where there are multiple intelligent inks and/or multiple agents, different sets of the microneedles 1006 may include tips 1006A with different intelligent inks and/or with different agents.

In addition, a length of the microneedles 1006, a volume of the tip 1006A and/or of the intelligent ink or agent infused thereon, or other aspect of the microneedles 1006 may vary depending on the intelligent ink or the agent. For example, FIG. 10A includes a detail view 1009 that illustrates microneedles 1006B-1006E with different lengths. The microneedle 1006B may include length and an intelligent ink or agent, the microneedle 1006C may include another length and another intelligent ink or agent, the microneedle 1006D may include another length and another intelligent ink or agent, and the microneedle 1006E may include another length and another intelligent ink or agent. For example, the microneedle 1006B may include a length in a range of 5-100 μm and a depigmentation agent infused in its tip 1006A. The microneedle 1006C may include a length in a range of 100-1000 μm and an intelligent ink infused in its tip 1006A. The microneedle 1006D may have a same length as the microneedle 1006C, e.g., a length in a range of 100-1000 μm, and another intelligent ink infused in its tip 1006A that is different than the intelligent ink of the microneedle 1006C. The microneedle 1006E may have a length in a range of 1000-3000 μm and a depilatory agent infused in its tip 1006A. The length of each of the microneedles 1006B-1006E may depend on the animal on which the tattoo applicator 1000 will be used (different animals may have different skin thicknesses), an injection site of the animal (different injection sites of an animal may have different skin thicknesses), the particular intelligent ink or agent included in the microneedles 1006B-1006E which is to be injected in the animal's skin, and/or other criteria.

Alternately or additionally, different ink elements may be included at different depths of the same microneedles. For example, detail view 1011 includes a microneedle 1006F with three different ink elements infused at three different depths of the needle 1006F. In particular, the tip 1006A of the microneedle 1006F may include the depilatory agent, a middle section 1006G of the microneedle 1006F may include one of the intelligent inks included in the microneedle 1006C or 1006D, and a base section 1006H of the microneedle 1006F may include the depigmentation agent. In other embodiments, different ink elements may be infused at the same or overlapping depths of the microneedle 1006F. More generally, microneedles with multiple ink elements may include two or more different ink elements at two or more different depths or at the same or overlapping depths of the microneedles. Such a configuration may allow each such microneedle to simultaneously deliver two or more different ink elements to two or more different layers or depths of the skin and/or to same or overlapping layers or depths of the skin.

In the example of FIG. 10A, one set of the microneedles 1006 of the tattoo applicator 1000 may include microneedles with a same configuration as the microneedle 1006B, another set of the microneedles 1006 may include microneedles with a same configuration as the microneedle 1006C, another set of the microneedles 1006 may include microneedles with a same configuration as the microneedle 1006D, and/or yet another set of the microneedles 1006 may include microneedles with a same configuration as the microneedle 1006E. The different sets of the needles 1006 may be separately located from each other or they may be intermixed. Further, although four sets of microneedles 1006 have been described in the context of the detail view 1009 of FIG. 10A, more generally the tattoo applicator 1000 may include one or more sets of microneedles 1006, with each set of microneedles 1006 having microneedles 1006 of a particular length and/or with a particular intelligent ink or agent.

The tattoo applicator 1000 may additionally include a substrate 1008 to which the microneedles 1006 are attached. The substrate 1008 may be rigid or flexible. A rigid substrate 1008 may be acceptable when, e.g., an injection site is relatively flat. A flexible substrate may be used when, e.g., the injection site is contoured to more uniformly cover the contoured injection site as compared to a rigid substrate. The substrate 1008 may be in the form of a patch.

Alternately or additionally, whether rigid or flexible, the substrate 1008 may be removable from the microneedles 1006. For example, after inserting the microneedles 1006 into the skin 1005 of the animal, the substrate may be peeled back from the microneedles 1006 or otherwise removed from the microneedles 1006, leaving the microneedles 1006 embedded in the skin 1005 to dissolve and release the intelligent inks and/or agents included therein.

An example process to apply a tattoo using the tattoo applicator 1000 will now be described with respect to FIG. 10B.

At stage 1010, the tattoo applicator 1000 may be manually or automatically positioned over a desired injection site. Lengths and ink elements of the microneedles 1006 may be configured to deliver a single ink element per microneedle 1006 or multiple ink elements per microneedle 1006 to a particular depth or depths of the skin 1005 based on thicknesses of layers of the skin at the injection site and the ink element or elements to be delivered by the microneedles 1006. For example, short microneedles 1006B, only one of which is labeled at stage 1010, with the depigmentation agent may be configured to deliver the depigmentation agent to epidermis 1005a of the skin 1005. Medium microneedles 1006C, only one of which is labeled at stage 1010, with the intelligent ink may be configured to deliver the intelligent ink to dermis 1005b of the skin 1005. Long microneedles 1006E, only one of which is labeled at stage 1010, with the depilatory agent may be configured to deliver the depilatory agent to a lower level of the dermis 1005b and/or to subcutis 1005c of the skin 1005. Alternatively or additionally, although not illustrated in FIG. 10B, microneedles 1006 with multiple ink elements, such as the microneedle 1006F of FIG. 10A, may be configured to deliver the depigmentation agent to the epidermis 1005a, the intelligent ink to the dermis 1005b, and the depilatory agent to the lower level of the dermis and/or to the subcutis 1005c.

At stage 1020, the microneedles 1006 are injected into the skin 1005.

At stage 1030, the substrate 1008 (see stages 1010 and 1020) is removed from the microneedles 1006. For example, the substrate 1008 may be peeled away from the skin 1005, with the microneedles 1006 breaking off of the substrate 1008 and remaining lodged in the skin 1005. While in the skin 1005, the microneedles 1006 may dissolve and/or be absorbed into the skin 1005.

At stage 1040, after the microneedles 1006 have partially or completely dissolved, the intelligent ink(s) and/or agent(s) delivered in the tips 1006A (FIG. 10A) of the microneedles 1006 may disperse in the skin 1005 generally at the depth they were injected by the microneedles 1006. For example, dispersion of the depigmentation agent delivered by the short microneedles 1006B is generally denoted at 1042. Dispersion of the intelligent ink delivered by the medium microneedles 1006C is generally denoted at 1044. Dispersion of the depilatory agent delivered by the long microneedles 1006E is generally denoted at 1046.

The tattoo applicator 1000 of FIGS. 10A and 10B may include other elements and/or configurations. For example, FIG. 10C illustrates another example configuration of the tattoo applicator 1000, arranged in accordance with at least some embodiments described herein. In FIG. 10C, the tattoo applicator 1000 includes a branding iron configuration. In particular, the tattoo applicator 1000 includes the microneedles 1006 and the substrate 1008. The tattoo applicator 1000 of FIG. 10C additionally includes a handle 1050 and a plate 1052 coupled to an end of the handle 1050. The substrate 1008 may be coupled to the plate 1052 with adhesive, tape, straps, a suction cup, or other suitable material or device.

In use, a rancher, ranch hand, or other person may attach the substrate 1008 with microneedles 1006 to the plate 1052. In some embodiments, the substrate 1008 includes a peel and stick backing on a back side of the substrate 1008 that is opposite a front side of the substrate 1008. The front side of the substrate 1008 includes the microneedles 1006. The peel and stick backing may include an adhesive on the back side of the substrate 1008 that is covered by a non-stick peelable layer. To attach the substrate 1008 to the plate 1052, the person may peel off the non-stick peelable layer to expose the adhesive and attach the substrate 1008 to the plate 1052 using the adhesive. Alternatively, other materials or devices may be used to attached the substrate 1008 to the plate 1052.

After the substrate 1008 with microneedles 1006 is attached to the plate 1052, the substrate 1008 with microneedles 1006 may be positioned at an injection site of an animal. The substrate 1008 with microneedles 1006 may be positioned at the injection site without touching the substrate 1008 with microneedles by grasping the handle 1050 and moving the plate 1052 and the attached substrate 1008 with microneedles 1006 to the injection site. When positioned at the injection site, the person may push the substrate with microneedles 1006 against the injection site using the handle 1050. The person may rotate or otherwise move the handle 1050 in a suitable manner to peel the substrate 1008 away from the injection site to break away from the microneedles 1006 and leave the microneedles 1006 within the animal's skin at the injection site. The person may then peel the remaining substrate 1008 off of the plate 1052 or otherwise remove the remaining substrate 1008 from the plate 1052 and discard it. The process may be repeated to apply tattoos to other animals, as desired.

FIG. 11 is a block diagram illustrating an example computing device 1100 that is arranged to monitor and/or tattoo an animal, arranged in accordance with at least some embodiments described herein. In a very basic configuration 1102, computing device 1100 typically includes one or more processors 1104 and a system memory 1106. A memory bus 1108 may be used for communicating between processor 1104 and system memory 1106.

Depending on the desired configuration, processor 1104 may be of any type including a microprocessor (μP), a microcontroller (μC), a digital signal processor (DSP), or any combination thereof. Processor 1104 may include one or more levels of caching, such as a level one cache 1110 and a level two cache 1112, a processor core 1114, and registers 1116. The example processor core 1114 may include an arithmetic logic unit (ALU), a floating point unit (FPU), a digital signal processing core (DSP Core), or any combination thereof. An example memory controller 1118 may also be used with processor 1104, or in some implementations memory controller 1118 may be an internal part of processor 1104.

Depending on the desired configuration, system memory 1106 may be of any type including volatile memory (such as RAM), nonvolatile memory (such as ROM, flash memory, etc.), or any combination thereof. System memory 1106 may include an operating system 1120, one or more applications 1122, and program data 1124. Application 1122 may include a health monitor application 1126 that may correspond to the health monitor application 112 of FIGS. 1A-1B and/or an application to control automatic or semiautomatic application of tattoos by a tattoo applicator. Program data 1124 may include animal records 1128 that may correspond to the animal records 114 of FIGS. 1A-1B. In some embodiments, application 1122 may be arranged to operate with program data 1124 on operating system 1120 to perform a method to monitor an animal, such as the method 400 of FIG. 4, and/or to perform other methods and/or operations described herein.

Computing device 1100 may have additional features or functionality, and additional interfaces to facilitate communications between basic configuration 1102 and any required devices and interfaces. For example, a bus/interface controller 1130 may be used to facilitate communications between basic configuration 1102 and one or more data storage devices 1132 via a storage interface bus 1134. Data storage devices 1132 may be removable storage devices 1136, non-removable storage devices 1138, or a combination thereof. Examples of removable storage and non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

System memory 1106, removable storage devices 1136, and non-removable storage devices 1138 are examples of computer storage media. Computer storage media includes RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other medium which may be used to store the desired information and which may be accessed by computing device 1100. Any such computer storage media may be part of computing device 1100.

Computing device 1100 may also include an interface bus 1140 for facilitating communication from various interface devices (e.g., output devices 1142, peripheral interfaces 1144, and communication devices 1146) to basic configuration 1102 via bus/interface controller 1130. Example output devices 1142 include a graphics processing unit 1148 and an audio processing unit 1150, which may be configured to communicate to various external devices such as a display or speakers via one or more A/V ports 1152. Example peripheral interfaces 1144 include a serial interface controller 1154 or a parallel interface controller 1156, which may be configured to communicate with external devices such as input devices (e.g., keyboard, mouse, pen, voice input device, touch input device, etc.), sensors, or other peripheral devices (e.g., printer, scanner, etc.) via one or more I/O ports 1158. An example communication device 1146 includes a network controller 1160, which may be arranged to facilitate communications with one or more other computing devices 1162 over a network communication link via one or more communication ports 1164.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term “computer-readable media” as used herein may include both storage media and communication media.

Computing device 1100 may be implemented as a portion of a small-form factor portable (or mobile) electronic device such as a cell phone, a personal data assistant, a tablet computer, a personal media player device, a wireless web-watch device, a personal headset device, an application-specific device, or a hybrid device that includes any of the above functions. Computing device 1100 may also be implemented as a personal computer including both laptop computer and non-laptop computer configurations. The computing device 1100 of FIG. 11 is an example implementation of the server 104, the tattoo reader 106, and/or the computing device 120 of FIGS. 1A-1B.

The many embodiments and examples described herein may be combined in any fashion desired. In some embodiments combining various aspects of the technology described herein, an antigen detection method may be combined with a temperature sensitive detection method, and injected into an animal skin using a microneedle array. The extent of combinations is left to the reader, and various combinations will become evident to those skilled in the art upon review of the present disclosure. A variety of non-limiting embodiments are now provided:

1. A tattoo ink, comprising:

an intelligent ink that includes a plurality of sensors, each of the plurality of sensors including:
a polydiacetylene (PDA) vesicle; and
a plurality of antigen receptors conjugated to the PDA vesicle;

wherein a variable optical property of each of the plurality of sensors is indicative of a presence or absence of a plurality of antigens specific to the plurality of antigen receptors.

2. The tattoo ink of embodiment 1, wherein the plurality of antigen receptors comprise a plurality of antibodies configured to bind the plurality of antigens, a plurality of antibody mimetics configured to bind the plurality of antigens, or a plurality of receptor molecules configured to bind the plurality of antigens.
3. The tattoo ink of embodiment 1, wherein:

the plurality of antigen receptors comprise a plurality of cortisol-specific antibodies and the plurality of antigens comprise a plurality of cortisol-specific antigens; or

the plurality of antigen receptors comprise a plurality of progesterone-specific antibodies and the plurality of antigens comprise a plurality of progesterone-specific antigens.

4. The tattoo ink of embodiment 1, further comprising at least one of:

epidermal growth factors;

an agent to promote healing and reduce scar formation at an injection site of the tattoo ink;

an opacity-enhancing agent; or

a hydrogel scaffold.

5. The tattoo ink of embodiment 1, wherein the PDA vesicle of each of the plurality of sensors is configured to undergo a conformational change responsive to the plurality of antigens binding to the plurality of antigen receptors of the PDA vesicle, the conformational change causing a change to the variable optical property of a corresponding one of the plurality of sensors.
6. The tattoo ink of embodiment 1, wherein the variable optical property of each of the plurality of sensors comprises at least one of a color or a fluorescence of each of the plurality of sensors.
7. The tattoo ink of embodiment 1, wherein each of the plurality of sensors further includes a semipermeable alginate encapsulant that encapsulates the PDA vesicle and the plurality of antigen receptors conjugated to the PDA vesicle.
8. The tattoo ink of embodiment 7, wherein the semipermeable alginate encapsulant is biocompatible.
9. The tattoo ink of embodiment 7, wherein the semipermeable alginate encapsulant is tuned to a molecular weight cutoff to allow passage of the plurality of antigens into a vicinity of the plurality of antigen receptors and to prevent ingress into a vicinity of the plurality of antigen receptors of enzymes that have a molecular weight above the molecular weight cutoff.
10. The tattoo ink of embodiment 1, wherein each of the plurality of sensors further includes a time-release encapsulant that encapsulates the PDA vesicle and the plurality of antigen receptors conjugated to the PDA vesicle.
11. The tattoo ink of embodiment 1, further comprising at least one of:

a depigmentation agent; or

a depilatory agent.

12. The tattoo ink of embodiment 11, wherein:

the depigmentation agent comprises monobenzone; or

the depilatory agent comprises thioglycolic acid.

13. A tattoo ink, comprising:

an intelligent ink that includes a plurality of sensors, each of the plurality of sensors including:
a leuco dye;
an organic acid;
a salt;
a solvent with a particular melting point; and
an encapsulant including the leuco dye, the organic acid, the salt, and the solvent;

wherein a variable optical property of each of the plurality of sensors is indicative of a temperature of each of the plurality of sensors.

14. The tattoo ink of embodiment 13, wherein the leuco dye comprises crystal violet lactone.
15. The tattoo ink of embodiment 13, wherein the encapsulant comprises a fatty alcohol mixture that includes myristyl alcohol and cetyl alcohol.
16. The tattoo ink of embodiment 13, wherein the variable optical property of each of the plurality of sensors comprises a color of each of the plurality of sensors that may vary between colorless in response to the temperature of a corresponding one of the plurality of sensors being less than the melting point and violet in response to the temperature of the corresponding one of the plurality of sensors being greater than the melting point.
17. A method to monitor an animal, the method comprising:

observing a tattoo of the animal, wherein the tattoo comprises an intelligent ink; and

determining a physiological status of the animal based on the tattoo.

18. The method of embodiment 17, further comprising, in response to the physiological status of the animal indicating a physiological event or condition of the animal, treating the animal for the physiological event or condition.
19. The method of embodiment 17, further comprising, in response to the physiological status of the animal indicating a physiological event or condition of the animal, separating the animal from other animals that do not have the physiological event or condition.
20. The method of embodiment 17, wherein observing the tattoo of the animal comprises viewing the tattoo of the animal with a naked eye.
21. The method of embodiment 17, wherein observing the tattoo of the animal comprises receiving light emitted, transmitted, and/or reflected by the intelligent ink at an optical imager.
22. The method of embodiment 17, wherein:

a color of the intelligent ink included in the tattoo is configured to respond to a particular target of physiology of the animal; and

determining the physiological status of the animal based on the tattoo includes determining the color of the intelligent ink,

23. A tattoo applicator, comprising:

a substrate;

a plurality of needles coupled to the substrate, wherein a first set of the plurality of needles have a first length and a second set of the plurality of needles have a second length that is different from the first length; and

an intelligent ink infused into at least some of the first set or the second set.

24. The tattoo applicator of embodiment 23, wherein the substrate comprises a flexible and peelable substrate.
25. The tattoo applicator of embodiment 23, wherein each of the plurality of needles comprises a self-dissolving and skin-absorbable needle.
26. The tattoo applicator of embodiment 23, wherein:

the intelligent ink is infused into each of the first set of the plurality of needles to be delivered to a first depth in skin of an animal; and

the tattoo applicator further includes a depigmentation agent or a depilatory agent infused into each of the second set of the plurality of needles to be delivered to a second depth in the skin of the animal that is different than the first depth.

27. The tattoo applicator of embodiment 23, wherein each of at least some of the plurality of needles includes:

the intelligent ink infused into a first portion of each of the at least some of the plurality of needles; and

another intelligent ink, a depigmentation agent, or a depilatory agent infused into a second portion of each of the at least some of the plurality of needles.

28. The tattoo applicator of embodiment 27, wherein each of the at least some of the plurality of needles is configured to:

deliver the intelligent ink to a first depth in skin of an animal; and

deliver the another intelligent ink, the depigmentation agent, or the depilatory agent to a second depth in the skin of the animal.

29. A tattoo applicator, comprising:

an ink delivery mechanism; and

a plurality of needles configured to:
interact with the ink delivery mechanism such that the ink delivery mechanism delivers ink to the needles; and
inject an area of skin with the ink after the ink is delivered to the needles from the ink delivery mechanism; the plurality of needles being arranged in a pattern according to a desired pattern of a tattoo.
   30. The tattoo applicator of embodiment 29, wherein the ink delivery mechanism comprises an ink pad configured to retain ink and the plurality of needles are configured to:

retain ink when passing through the ink pad;

pass through the ink pad to retain the ink of the ink pad; and

inject the area of the skin with the ink after passing through the ink pad.

31. The tattoo applicator of embodiment 29, wherein the ink delivery mechanism comprises a roller configured to deliver ink onto an epidermal surface of the skin at the area and wherein the plurality of needles are configured to inject the area with the ink after the roller delivers the ink onto the area.
32. The tattoo applicator of embodiment 29, wherein the desired pattern includes one or more of: a number, a letter, a shape, a picture, and a barcode.
33. The tattoo applicator of embodiment 29, wherein the ink comprises an intelligent ink
34. The tattoo applicator of embodiment 29, wherein the plurality of needles are configured to inject the ink to a dermal layer of the skin or to a subcutaneous layer of the skin.
35. The tattoo applicator of embodiment 29, further comprising a sanitation mechanism configured to retain a sanitation agent and configured to interact with the plurality of needles such that the sanitation agent comes in contact with the plurality of needles after injection of the ink into the area.
36. The tattoo applicator of embodiment 35, wherein the plurality of needles include a hollow portion and the sanitation mechanism is configured to deliver the sanitation agent through the hollow portion.
37. The tattoo applicator of embodiment 29, further comprising an ink reservoir configured to retain the ink and to provide the ink to the ink delivery mechanism.
38. The tattoo applicator of embodiment 29, wherein the ink includes at least two ink types having different properties and the ink delivery mechanism is configured such that different ink types are delivered to different portions of the area.
39. The tattoo applicator of embodiment 29, further comprising a cleansing mechanism configured to administer a cleansing agent to the area before injection of the ink.
40. The tattoo applicator of embodiment 29, further comprising a pattern selection mechanism configured to change the pattern of the plurality of needles.
41. A tattoo applicator, comprising:

a roller configured to deliver ink onto an area of an epidermal surface of skin; and

a plurality of needles configured to inject the skin with the ink at the area after the roller delivers the ink onto the area; the plurality of needles being arranged in a pattern according to a desired pattern of a tattoo.

42. The tattoo applicator of embodiment 41, wherein the desired pattern includes one or more of: a number, a letter, a shape, a picture, and a barcode.
43. The tattoo applicator of embodiment 41, wherein the ink comprises an intelligent ink.
44. The tattoo applicator of embodiment 41, wherein the plurality of needles are configured to inject the ink to a dermal layer of the skin or to a subcutaneous layer of the skin.
45. The tattoo applicator of embodiment 41, further comprising a sanitation mechanism configured to retain a sanitation agent and configured to interact with the roller such that the sanitation agent comes in contact with the roller after delivery of the ink onto the area.
46. The tattoo applicator of embodiment 41, further comprising a sanitation mechanism configured to retain a sanitation agent and configured to interact with the plurality of needles such that the sanitation agent comes in contact with the plurality of needles after injection of the ink at the area.
47. The tattoo applicator of embodiment 46, wherein the plurality of needles include a hollow portion and the sanitation mechanism is configured to deliver the sanitation agent through the hollow portion.
48. The tattoo applicator of embodiment 41, further comprising an ink reservoir configured to retain the ink and to provide the ink to the roller.
49. The tattoo applicator of embodiment 41, wherein the ink includes at least two ink types having different properties and the roller is configured to deliver the at least two ink types to the area such that different ink types are delivered to different portions of the area.
50. The tattoo applicator of embodiment 41, further comprising a cleansing mechanism configured to administer a cleansing agent to the area before the roller delivers the ink to the area.
51. The tattoo applicator of embodiment 41, further comprising a pattern selection mechanism configured to change the pattern of the plurality of needles.
52. A tattoo applicator, comprising:

an ink pad configured to retain ink; and

a plurality of needles configured to:
retain ink when passing through the ink pad;
pass through the ink pad to retain the ink of the ink pad; and
inject an area of skin with the ink after passing through the ink pad; the plurality of needles being arranged in a pattern according to a desired pattern of a tattoo.
   53. The tattoo applicator of embodiment 52, wherein the desired pattern includes one or more of: a number, a letter, a shape, a picture, and a barcode.
   54. The tattoo applicator of embodiment 52, wherein the ink comprises an intelligent ink.
   55. The tattoo applicator of embodiment 52, wherein the plurality of needles are configured to inject the ink to a dermal layer of the skin or to a subcutaneous layer of the skin.
   56. The tattoo applicator of embodiment 52, further comprising a sanitation mechanism configured to retain a sanitation agent and configured to interact with the plurality of needles such that the sanitation agent comes in contact with the plurality of needles after injection of the ink into the area.
   57. The tattoo applicator of embodiment 56, wherein the plurality of needles include a hollow portion and the sanitation mechanism is configured to deliver the sanitation agent through the hollow portion.
   58. The tattoo applicator of embodiment 52, further comprising an ink reservoir configured to retain the ink and to provide the ink to the ink pad.
   59. The tattoo applicator of embodiment 52, wherein the ink includes at least two ink types having different properties and the ink pad is configured to retain the at least two ink types in different portions of the ink pad such that that different ink types are delivered to different portions of the area.
   60. The tattoo applicator of embodiment 52, further comprising a cleansing mechanism configured to administer a cleansing agent to the area of the skin before injection of the ink.
   61. The tattoo applicator of embodiment 52, further comprising a pattern selection mechanism configured to change the pattern of the plurality of needles.

The present disclosure is not to be limited in terms of the particular embodiments described herein, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. It is to be understood that the present disclosure is not limited to particular methods, reagents, compounds compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub ranges and combinations of sub ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into sub ranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, various embodiments of the present disclosure have been described herein for purposes of illustration, and various modifications may be made without departing from the scope and spirit of the present disclosure. Accordingly, the various embodiments disclosed herein are not intended to be limiting, with the true scope and spirit being indicated by the following claims.

1. A method to monitor an animal, the method comprising:
determining a physiological status of the animal based on a tattoo that includes intelligent ink injected into skin of the animal,
recording the physiological status of the animal determined based on the tattoo; and
in response to the physiological status indicating a physiological event or condition, triggering an alert to address the physiological event or condition.

2. The method of claim 1, wherein determining the physiological status of the animal based on the tattoo that includes the intelligent ink injected into the skin of the animal comprises:
receiving light transmitted, emitted, and/or reflected by the intelligent ink;
determining a current value of an optical property of the intelligent ink based on the received light; and
comparing the current value of the optical property to a baseline value.

3. The method of claim 2, wherein determining the physiological status of the animal based on the tattoo that includes the intelligent ink injected into the skin of the animal further comprises:
determining that the physiological status of the animal with respect to a particular target of physiology to which the intelligent ink is configured to respond is normal in response to the current value of the optical property being within a particular range of the baseline value; or
determining that the physiological status of the animal with respect to the particular target is abnormal in response to the current value of the optical property being outside the particular range of the baseline value.

4. The method of claim 2, wherein:
determining the physiological status of the animal based on the tattoo that includes the intelligent ink injected into the skin of the animal further comprises illuminating the intelligent ink with ultraviolet light; and
determining the current value of the optical property of the intelligent ink based on the received light comprises determining an optical property associated with fluorescence emitted by the intelligent ink in response to absorption by the intelligent ink of at least some of the ultraviolet light.

5. The method of claim 1, wherein triggering an alert to address the physiological event or condition comprises generating an electronic message that identifies the physiological event or condition and the animal from among a plurality of animals that each have a tattoo with intelligent ink injected into skin of the corresponding one of the plurality of animals.

6-7. (canceled)

8. The method of claim 1, wherein:
an optical property of the intelligent ink depends on a particular target of physiology of the animal;
determining the physiological status of the animal based on the tattoo that includes intelligent ink injected into the skin of the animal comprises determining a value of the optical property of the intelligent ink; and
the particular target of physiology of the animal comprises a hydration level of the animal, a temperature of the animal, a pH level of the animal, a level of a particular hormone in the animal, or a particular pathogen in the animal.

9. (canceled)

10. The method of claim 1, wherein:
the tattoo includes a plurality of intelligent inks injected into the skin of the animal;
each of the plurality of intelligent inks depends on a different one of a plurality of targets of physiology of the animal; and
determining the physiological status of the animal based on the tattoo that includes the intelligent ink injected into the skin of the animal comprises determining the physiological status of the animal with respect to each of the plurality of targets of physiology of the animal.

11. The method of claim 1, further comprising applying the tattoo to the animal, including:
positioning a flexible substrate with a plurality of needles at an injection site of the animal, wherein each of the plurality of needles comprises a self-dissolving and skin-absorbable needle and wherein at least some of the plurality of needles include the intelligent ink infused in a tip of each of the at least some of the plurality of needles;
injecting the plurality of needles into the skin at the injection site;
removing the substrate from the plurality needles while the plurality needles remain lodged in the skin; and
dissolving the at least some of the plurality of needles to release the intelligent ink infused in the tip of each of the at least some of the plurality of needles.

12. The method of claim 12, wherein another at least some of the plurality of needles include at least one of a depilatory agent or a depigmentation agent infused in a tip of each of the another at least some of the plurality of needles such that applying the tattoo to the animal further includes injecting at least one of the depilatory agent or the depigmentation agent into the skin at the injection site.

13. A system to monitor an animal, the system comprising:
a tattoo reader configured to measure an optical property of an intelligent ink injected into skin of the animal as a tattoo and to generate a current value of the measured optical property;
a processor communicatively coupled to the tattoo reader;
a computer-readable medium communicatively coupled to the processor; and
a health monitor application comprising computer instructions executable by the processor to perform operations comprising:
determining a physiological status of the animal based on the current value of the measured optical property of the intelligent ink;
recording the physiological status of the animal determined based on the current value of the measured optical property of the intelligent ink; and
in response to the physiological status indicating a physiological event or condition, triggering an alert to address the physiological event or condition.

14-15. (canceled)

16. The system of claim 13, wherein:
the tattoo reader comprises an optical imager configured to measure the optical property, generate the current value by receiving light transmitted, emitted, and/or reflected by the intelligent ink, and generate an electrical signal representing the optical property;
the tattoo reader further comprises a radiation source configured to emit radiation; and
at least one of:
the received light comprises at least some of the radiation after reflection by the intelligent ink; and
the received light comprises fluorescence emitted by the intelligent ink in response to absorption by the intelligent ink of at least some of the radiation.

17-18. (canceled)

19. The system of claim 13, wherein the optical property of the intelligent ink is configured to undergo an irreversible change in response to a particular target of physiology of the animal satisfying a particular criteria at least once, and wherein the irreversible change is measurable whether or not the particular criteria is satisfied at a time of measurement.

20. The system of claim 13, wherein the optical property of the intelligent ink is configured to undergo a reversible change in response to a particular target of physiology of the animal satisfying a particular criteria, and wherein the reversible change is not measurable unless the particular criteria is satisfied at a time of measurement.

21. The system of claim 13, further comprising a plurality of animal records stored in the computer-readable medium, wherein each of the plurality of animal records corresponds to a different one of a plurality of animals and wherein each of the plurality of animal records includes multiple physiological status entries recorded over time for a corresponding one of the plurality of animals.

22. A computer-readable medium that includes computer-readable instructions stored thereon, which in response to execution by a processor, cause the processor to perform or cause the processor to control performance of operations comprising:
determining a physiological status of an animal based on a tattoo that includes intelligent ink injected into skin of the animal,
recording the physiological status of the animal determined based on the tattoo; and
in response to the physiological status indicating a physiological event or condition, triggering an alert to address the physiological event or condition.

23. The computer-readable medium of claim 22, wherein determining the physiological status of the animal based on the tattoo that includes the intelligent ink injected into the skin of the animal comprises:
receiving light transmitted, emitted, and/or reflected by the intelligent ink;
determining a current value of an optical property of the intelligent ink based on the received light; and
comparing the current value of the optical property to a baseline value.

24. The computer-readable medium of claim 23, wherein determining the physiological status of the animal based on the tattoo that includes the intelligent ink injected into the skin of the animal further comprises:
determining that the physiological status of the animal with respect to a particular target of physiology to which the intelligent ink is configured to respond is normal in response to the current value of the optical property being within a particular range of the baseline value; or
determining that the physiological status of the animal with respect to the particular target is abnormal in response to the current value of the optical property being outside the particular range of the baseline value.

25. The computer-readable medium of claim 23, wherein:
determining the physiological status of the animal based on the tattoo that includes the intelligent ink injected into the skin of the animal further comprises illuminating the intelligent ink with ultraviolet light; and
determining the current value of the optical property of the intelligent ink based on the received light comprises determining an optical property associated with fluorescence emitted by the intelligent ink in response to absorption by the intelligent ink of at least some of the ultraviolet light.

26. The computer-readable medium of claim 22, wherein triggering an alert to address the physiological event or condition comprises generating an electronic message that identifies the animal and the physiological event or condition.

27-28. (canceled)

29. The computer-readable medium of claim 22, wherein:
an optical property of the intelligent ink depends on a particular target of physiology of the animal;
determining the physiological status of the animal based on the tattoo that includes intelligent ink injected into the skin of the animal comprises determining a value of the optical property of the intelligent ink; and
the particular target of physiology of the animal comprises a hydration level of the animal, a temperature of the animal, a pH level of the animal, a level of a particular hormone in the animal, or a particular pathogen in the animal.

30. (canceled)

31. The computer-readable medium of claim 22, wherein:
the tattoo includes a plurality of intelligent inks injected into the skin of the animal;
each of the plurality of intelligent inks depends on a different one of a plurality of targets of physiology of the animal; and
determining the physiological status of the animal based on the tattoo that includes the intelligent ink injected into the skin of the animal comprises determining the physiological status of the animal with respect to each of the plurality of targets of physiology of the animal.

32. The method of claim 1, wherein:
the intelligent ink comprises a plurality of sensors;
each of the plurality of sensors includes a polydiacetylene (PDA) vesicle and a plurality of antigen receptors conjugated to the PDA vesicle; and
a variable optical property of each of the plurality of sensors is indicative of a presence or absence of a plurality of antigens specific to the plurality of antigen receptors.

33. The method of claim 1, wherein:
the intelligent ink comprises a plurality of sensors;
each of the plurality of sensors includes
a leuco dye;
an organic acid;
a salt;
a solvent with a particular melting point; and
an encapsulant including the leuco dye, the organic acid, the salt, and the solvent; and
a variable optical property of each of the plurality of sensors is indicative of a temperature of each of the plurality of sensors.

34. The method of claim 1, wherein:
the intelligent ink includes an optical property configured to undergo an irreversible change in response to a particular target of physiology of the animal satisfying a particular criteria at least one;
determining the physiological status of the animal based on the tattoo includes determining that the particular criteria has been satisfied at least once based on a current value of the optical property of the intelligent ink that is measured at a time when the particular criteria is not satisfied.
